(19) United States
(12) Patent Application Publication
(10) Pub. No.: US 2002/0025948 A1
(43) Pub. Date: Feb. 28, 2002

(54) 3-azabicyclo[3.1.0]hexane derivatives useful in therapy

(76) Inventors: Bernard Joseph Banks, Kent (GB); Douglas James Critcher, Kent (GB); Ashley Edward Fenwick, Kent (GB); David Morris Gethin, Kent (GB); Stephen Paul Gibson, Kent (GB)

Correspondence Address: Paul H. Ginsburg, Pfizer Inc, 20th Floor, 235 East 42nd Street, New York, NY, 10017-5755, US

(21) Appl. No.: 09/883,567

(22) Filed: Jun. 18, 2001

Related U.S. Application Data
(60) Provisional application No. 60/218,500, filed on Jul. 14, 2000.

(30) Foreign Application Priority Data
Jun. 23, 2000 (GB) 00 15562.2

(51) Int. Cl. A61K 31/675; A61K 31/4439; A61K 31/423; A61K 31/403; C07D 403/02

(52) U.S. Cl. 514/79; 514/339; 514/397; 514/394; 514/412; 546/276.7; 548/452; 548/412; 548/312.1; 548/305.1

(57) ABSTRACT

Compounds of formula I,

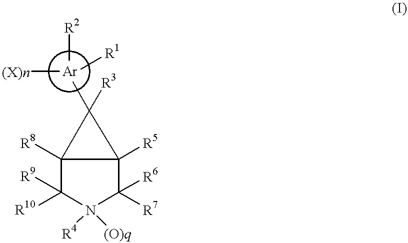

where the substituents are as defined herein, and the pharmaceutically or veterinarily acceptable derivatives or prodrugs thereof, are pharmaceutically and veterinarily useful, in particular they bind to opiate receptors (e.g. mu, kappa and delta opioid receptors). They are likely to be useful in the treatment of diseases or conditions modulated by opiate receptors, for example irritable bowel syndrome; constipation; nausea; vomiting; pruritic dermatoses, such as allergic dermatitis and atopy; eating disorders; opiate overdoses; depression; smoking and alcohol addiction; sexual dysfunction; shock; stroke; spinal damage; and head trauma.

This invention relates to pharmaceutically useful compounds, in particular compounds that bind to opiate receptors (e.g. mu, kappa and delta opioid receptors). Compounds that bind to such receptors are likely to be useful in the treatment of diseases modulated by opiate receptors, for example irritable bowel syndrome; constipation; nausea; vomiting; and pruritic dermatoses, such as allergic dermatitis and atopy in animals and humans. Compounds that bind to opiate receptors have also been indicated in the treatment of eating disorders, opiate overdoses, depression, smoking and alcohol addiction, sexual dysfunction, shock, stroke, spinal damage and head trauma.

There is a particular need for an improved treatment of itching. Itching, or pruritus, is a common dermatological symptom that can give rise to considerable distress in both humans and animals. Pruritus is often associated with inflammatory skin diseases which may be caused by hypersensitivity reactions, including reactions to insect bites, such as flea bites, and to environmental allergens, such as house dust mite or pollen; by bacterial and fungal infections of the skin; or by ectoparasite infections.

Existing treatments that have been employed in the treatment of pruritus include the use of corticosteroids and antihistamines. However, both of these treatments are known to have undesirable side effects. Other therapies that have been employed include the use of essential fatty acid dietary supplements, though these have the disadvantages of being slow to act, and of offering only limited efficacy against allergic dermatitis. A variety of emollients such as soft paraffin, glycerine and lanolin are also employed, but with limited success.

Thus, there is a continuing need for alternative and/or improved treatments of pruritus.

Certain 4-arylpiperidine-based compounds are disclosed in inter alia European patent applications EP 287339, EP 506468 and EP 506478 as opioid antagonists. In addition, International Patent Application WO 95/15327 discloses azabicycloalkane derivatives useful as neuroleptic agents.

International Patent Application WO00/39089, filed before the priority date of the instant application, but published thereafter, is herein incorporated by reference in its entirety, and discloses azabicycloalkanes of similar structure to those described herein below, with different R⁴ groups.

According to the invention there is provided a compound of formula I,

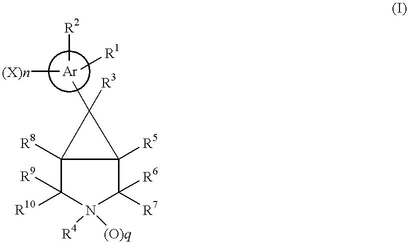

wherein the “Ar” ring represents an optionally benzo-fused phenyl or 5- or 6-membered heteroaryl ring;

R¹ when taken alone is H, halogen, NO₂, NH₂, NY²WY¹, Het¹, AD, CO₂R⁷, C(O)R⁸, C(═NOH)R⁸, or OE,

Y² is H, C₁₋₆ alkyl, C₃₋₆ alkenyl (each of which alkyl and alkenyl is optionally substituted by aryl, aryloxy or Het¹),

W is SO₂, CO, C(O)O, P(Y¹)═O, P(Y¹)═S,

Y¹ is C₁₋₁₀ alkyl (optionally substituted by one or more substituents independently selected from halogen, OH, C₁₋₄ alkoxy, C₁₋₆ alkanoyloxy, CONH₂, C₁₋₆ alkoxycarbonyl, NH₂, aryl, mono- or di(C₁₋₄ alkyl)amino, C₃₋₈ cycloalkyl, phthalimidyl, Het¹), Het¹, aryl (optionally substituted by one or more substituents independently selected from C₁₋₄ alkyl, C₁₋₄ haloalkyl and halogen), NH₂, N(C₁₋₄ alkyl)₂ or NH(C₁₋₆ alkyl),

Het¹ is a heterocyclic group containing up to 4 heteroatoms selected from N, O and S, which may comprise up to 3 rings (preferably a heteroaryl group, optionally benzo- or pyrido-fused heteroaryl), optionally substituted by one or more substituents independently selected from C₁₋₆ alkyl, C₁₋₆ alkoxy, C₃₋₆ cycloalkyl, C₁₋₆ haloalkoxy, C₁₋₆ haloalkyl, C₃₋₆ halocycloalkyl, ═O, OH, halogen, NO₂, SiR^{19a}R^{19b}R^{19c}, CON^{20a}R^{20b}, NR₂₀ₐR^{20b}, SR^{21a}, NR^{21b}SO₂R^{22a}, NR^{21c}C(O)OR^{22b}, NR^{21d}COR^{22d}, and C₁₋₆ alkoxycarbonyl,

and if a S atom is present in a ring, it can be present as part of a —S—, S(O)— or —S(O₂)— group, and carbon atoms in the ring can be present as a part of a carbonyl moiety;

R^{19a}, R^{19b}, R^{19c} each independently represent C₁₋₆ alkyl or aryl,

R^{20a} and R^{20b} each independently represent H, C₁₋₆ alkyl, aryl, (C₁₋₄ alkyl)phenyl, each of which alkyl, aryl and alkylphenyl are optionally substituted by one or more C₁₋₄ alkyl, C₁₋₄ alkoxy, OH, NO₂, NH₂ and/or halogen,

or R^{20a} and R^{20b} can be taken together with the N atom to which they are attached, to form a 4- to 6-membered ring optionally substituted by one or more substitutuents independently selected from one or more C₁₋₄ alkyl, C₁₋₄ alkoxy, OH, ═O, NO₂, NH₂ and/or halogen,

R^{21a, b, c and d} each independently represent H, C_{1 6} alkyl, aryl or C₁₋₄ alkylphenyl, each of which alkyl, aryl, and alkylphenyl are optionally substituted by one or more C₁₋₄ alkyl, C₁₋₄ alkoxy, OH, NO₂, halogen, NH₂,

R^{22a, b and c} each independently represent C₁₋₆ alkyl, aryl or C₁₋₄ alkylphenyl, each of which alkyl, aryl, and alkylphenyl are optionally substituted by one or more C₁₋₄ alkyl, C₁₋₄ alkoxy, OH, NO₂, halogen, NH₂,

A is C₁₋₄ alkylene, C₂₋₄ alkenylene or C₂₋₄ alkynylene, each of which is optionally substituted by one or more C₁₋₄ alkyl, C₁₋₄ alkoxy, halogen and/or OH,

D is H, OH, CN, NR²⁵R²⁶, CONR²⁵R²⁶ , NHR²⁷ CO₂R²⁸, COR²⁹, C(═NOH)R²⁹,

or AD is CN, NR²⁵R²⁶, CONR²⁵R²⁶,

where R²⁵ and R²⁶ are either each independently H, C₁₋₃ alkyl, C₃₋₈ cycloalkyl, aryl, C₁₋₄ alkylphenyl (each of which C₁₋₃ alkyl, C₃₋₈ cycloalkyl, aryl and C₁₋₄ alkylphenyl are optionally substituted by one or more NO₂, halogen, C₁₋₄ alkyl and/or C₁₋₄ alkoxy, (each of which latter C₁₋₄ alkyl and C₁₋₄ alkoxy is optionally substituted by one or more halogen)),

or R²⁵ and R²⁶ are taken together with the N atom to which they are attached and can form a 4- to 7-membered heterocyclic ring optionally incorporating one or more further hetero atoms selected from N, O and S, and which ring is optionally substituted by one or more C₁₋₄ alkyl, OH, ═O, NO₂, NH₂ and/or halogen,

R²⁷ is COR³⁰, CO₂R^{31a}, SO₂R^{31b},

R²⁸ and R²⁹ are each independently H, C₁₋₆ alkyl, C₃₋₈ cycloalkyl, aryl or C₁₋₄alkylphenyl, each of which C₁₋₆ alkyl, C₃₋₈ cycloalkyl, aryl and C₁₋₄ alkylphenyl are optionally substituted by one or more NO₂, halogen, C₁₋₄ alkyl, C₁₋₄ alkoxy (each of which latter C₁₋₄ alkyl and C₁₋₄ alkoxy are optionally substituted by one or more halogen),

R³⁰ is H, C₁₋₄ alkyl, C₃₋₈ cycloalkyl, C₁₋₄ alkoxy, C₃₋₈ cycloalkyloxy, aryl, aryloxy, C₁₋₄ alkylphenyl, phenyl(C₁₋₄ )alkoxy, (each of which C₁₋₄ alkyl, C₃₋₈ cycloalkyl, C₁₋₄ alkoxy, C₃₋₈ cycloalkyloxy, aryl, aryloxy, C₁₋₄ alkylphenyl and phenyl(C₁₋₄)alkoxy are optionally substituted by one or more NO₂, halogen, C₁₋₄ alkyl, C₁₋₄ alkoxy (which latter alkyl and alkoxy are optionally substituted by one or more halogen)),

R^{31a} and R^{31b} are each independently C₁₋₄ alkyl, C cycloalkyl, aryl or C₁₋₄ alkylphenyl, each of which is optionally substituted by one or more NO₂, halogen, C₁₋₄ alkyl or C₁₋₄ alkoxy, each of which latter alkyl and alkoxy is optionally substituted by one more halogen

E is H, CONR³²R³³, CSNR³²R³³, COR³⁴, CO₂R³⁴, COCH(R^{34a})NH₂, R³⁵, CH₂CO₂R^{35a}, CHR^{35b}CO₂R^{35a}, CH₂OCO₂R^{35c}, CHR^{35d}OCO₂R^{35c}, COCR³⁶═CR³⁷NH₂, COCHR³⁶CHR³⁷NH₂, or PO(OR³⁸)₂,

R³² and R³³ are each independently H, C₃₋₁₀ alkylalkenyl, C₃₋₇ cycloalkyl (optionally substituted by C₁₋₄ alkyl), phenyl (optionally substituted by (X)ₙ), C₁₋₁₀ alkyl (optionally substituted by C₄₋₇ cycloalkyl (optionally substituted by C₁₋₄ alkyl) or phenyl optionally substituted by (X)ₙ),

or R³² and R³³ can be taken together with the N atom to which they are attached and can form a 5- to 8-membered heterocycle optionally comprising further hetero atoms selected from N, O and S, which heterocycle is optionally substituted by C₁₋₄ alkyl, optionally substituted by one or more halogen,

R³⁴ is H, C₄₋₇ cycloalkyl (optionally substituted by one or more C₁₋₄ alkyl), phenyl (optionally substituted by (X)ₙ, C₁₋₄ alkanoyloxy, NR³²R³³, CONR³²R³³ and/or OH), or C₁₋₆ alkyl (optionally substituted by one or more halogen, C₄₋₇ cycloalkyl (optionally substituted by one or more C₁₋₄ alkyl), or phenyl (optionally substituted by (X)ₙ,C₁₋₄ alkanoyloxy, NR³²R³³, CONR³²R³³ and/or OH)),

R^{34a} is H, C₁₋₆ alkyl (optionally substituted by one or more halogen, C₄₋₇ cycloalkyl (optionally substituted by one or more C₁₋₄ alkyl), or phenyl (optionally substituted by (X)ₙ, C₁₋₄ alkanoyloxy, NR³²R³³, CONR³²R³³ and/or OH)), C₄₋₇ cycloalkyl (optionally substituted by one or more C₁₋₄ alkyl), phenyl (optionally substituted by (X)ₙ,C₁₋₄ alkanoyloxy, NR³²R³³, CONR³²R³³ and/or OH) or a naturally occuring amino acid substituent,

R³⁵ is C₄₋₇ cycloalkyl optionally substituted by one or more C₁₋₄ alkyl, phenyl (optionally substituted by one or more (X)ₙ, C₁₋₄ alkanoyl, NHR³², CON(R³²)₂, and/or OH), C₁₋₆ alkyl (optionally substituted by C₄₋₇ cycloalkyl optionally substituted by one or more C₁₋₄ alkyl, or phenyl (optionally substituted by one or more (X)ₙ, C₁₋₄ alkanoyl, NHR³², CON(R³²)₂, and/or OH)), C₁₋₄ alkoxy(C₁₋₄ alkyl), phenyl(C₁₋₄)alkyloxy(C₁₋₄)alkyl, tetrahydropyranyl, tetrahydrofuranyl, cinnamyl or trimethylsilyl,

R^{35a,b,c and d} are each independently H, C₄₋₇ cycloalkyl optionally substituted by one or more C₁₋₄ alkyl, phenyl optionally substituted by one or more (X)ₙ or C₁₋₆ alkyl (optionally substituted by C₄₋₇ cycloalkyl optionally substituted by one or more C₁₋₄ alkyl, or phenyl optionally substituted by one or more (X)ₙ),

R³⁶ and R³⁷ each independently represent H, C₃₋₆ alkylalkenyl, C₄₋₇ cycloalkyl, phenyl optionally substituted by one or more (X)ₙ, or C₁₋₆ alkyl (optionally substituted by C₄₋₇ cycloalkyl optionally substituted by one or more C₁₋₄ alkyl, or phenyl optionally substituted by one or more (X)ₙ),

R³⁸ is C₄₋₇ cycloalkyl optionally substituted by one or more C₁₋₄ alkyl, phenyl optionally substituted by one or more (X)ₙ, or C₁₋₆ alkyl (optionally substituted by C₄₋₇ cycloalkyl optionally substituted by one or more C₁₋₄ alkyl, or phenyl optionally substituted by one or more (X)ₙ),

R² when taken alone is H or halogen;

or R¹ and R², when attached to adjacent carbon atoms, can be taken together with the carbon atoms to which they are attached, and may represent Het^{1a};

Het^{1a} is a heterocyclic group containing up to 4 heteroatoms selected from N, O and S, which may comprise up to 3 rings (and is preferably an optionally benzo-fused 5- to 7-membered heterocyclic ring) and which group is optionally substituted by one or more substituents independently selected from OH, ═O, halogen, C₁₋₄ alkyl, C₁₋₄ haloalkyl, C₁₋₄ alkoxy and C₁₋₄ haloalkoxy,

which C₁₋₄ alkyl, C₁₋₄ haloalkyl, C₁₋₄ alkoxy and C₁₋₄ haloalkoxy groups can be optionally substituted by one or more C₃₋₆ cycloalkyl, aryl(C₁₋₆)alkyl,

which aryl group is optionally substituted by one or more halogen, C₁₋₄ alkyl, C₁₋₄ haloalkyl, C₁₋₄ alkoxy and C₁₋₄ haloalkoxy,

which latter C₁₋₄ alkyl, C₁₋₄ haloalkyl, C₁₋₄ alkoxy and C₁₋₄ haloalkoxy groups can be optionally substituted by one or more NR²³R²⁴, NR²³S(O)ₙR²⁴, NR²³C(O)ₘR²⁴,

and if a S atom is present in a ring, it can be present as part of a —S—, S(O)— or —S(O₂)— group,

which R²³ and R²⁴ when taken alone independently represent H, C₁₋₄ alkyl, or C₁₋₄ haloalkyl,

or R²³ and R²⁴ can be taken together with the N atom to which they are attached, to form a 4- to 6-membered heterocyclic ring optionally comprising one or more further heteroatoms selected from, N, O, or S, and which heterocyclic ring is optionally substituted by one or more halogen, C₁₋₄ alkyl, C₁₋₄ haloalkyl, C₁₋₄ alkoxy and/or C₁₋₄ haloalkoxy groups,

R³ is H, CN, halogen, C₁₋₄ alkoxy, C₁₋₄ alkoxycarbonyl, C₂ alkanoyl, C₂₋₆ alkanoyloxy, C₃₋₈ cycloalkyl, C₃₋₈ cycloalkyloxy, C₄₋₉ cycloalkanoyl, aryl, aryloxy, heteroaryl, saturated heterocycle, NR¹²R¹³, CONR¹²R¹³, NY²WY¹, C₁₋₆ alkyl, C₂₋₁₀ alkenyl, C₂₋₁₀ alkynyl, (each of which alkyl, alkenyl and alkynyl groups is optionally substituted by one or more CN, halogen, OH, C₁₋₆ alkoxy, C₁₋₆ alkoxycarbonyl, C₂₋₆ alkyloxycarbonyloxy, C₁₋₆ alkanoyl, C₁₋₆ alkanoyloxy, C₃₋₈ cycloalkyl, C₃₋₈ cycloalkyloxy, C₄₋₉ cycloalkanoyl, aryl, aryloxy, heteroaryl, saturated heterocycle, NR¹²R¹³, CONR¹²R¹³ and/or NY²WY¹),

R⁴ is C₁₋₁₀ alkyl, C₃₋₁₀ alkenyl or C₃₋₁₀ alkynyl, each of which groups is linked to the N atom via a sp³ carbon, and which group is substituted by one or more substituents selected from:

C₂₋₆ alkoxy [substituted by one or more groups selected from OH, NR²⁵R²⁶, CONR²⁵R²⁶, halogen, C₁₋₆ alkoxy, C₂₋₄ alkynyl, C₂₋₄ alkenyl, heteroaryl¹, aryl¹, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O),(C₁alkyl), S(O),(aryl¹), S(O),(heteroaryl¹), SO₂NR²⁵R²⁶ and cycloalkyl¹],

S(O)ₙC₁₋₆alkyl [optionally substituted by one or more groups selected from OH, NR²⁵R²⁶, CONR²⁵R²⁶, halogen, C₁₋₆ alkoxy, C₂₋₄ alkynyl, C₂₋₄ alkenyl, heteroaryl¹, aryl¹, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O)ₙ(C₁₋₆ alkyl), S(O)ₙ(aryl¹), S(O)ₙ(heteroaryl¹), SO₂NR²⁵R²⁶ and cycloalkyl¹],

aryl²,

CO₂CH₂(heteroaryl¹),

CO₂CH₂(aryl¹),

cycloalkyl¹,

CO(heteroaryl¹),

CO(aryl¹),

OCO(aryl¹),

OCO(heteroaryl¹),

OCO(C₁alkyl),

OCOCH₂CN,

CO₂(heteroaryl¹),

CO₂(aryl¹),

COCH₂(heteroaryl¹),

S(O)ₙCH₂aryl¹,

S(O)ₙ(heteroaryl¹),

S(O)ₙCH₂(heteroaryl¹),

NHSO₂aryl¹,

NHSO₂(C₁₋₆ alkyl),

NHSO₂(heteroaryl¹),

NHSO₂CH₂(heteroaryl¹),

NHSO₂CH₂(aryl¹),

NHCOaryl¹,

NHCO(C₁alkyl),

NHCONHaryl¹,

NHCONH(C₁₋₆ alkyl),

NHCOheteroaryl¹,

NHCONHheteroaryl¹,

NHCO₂(aryl¹),

NHCO₂(C₁₋₆ alkyl),

NHCO₂(heteroaryl¹),

aryl²oxy,

heteroaryl¹oxy,

C₁₋₄ alkoxycarbonyl substituted by C₁₋₆ alkyl, aryl, C₁₋₆ alkoxy, CH₂(aryl¹), C₁₋₄ haloalkyl, halogen, OH, CN or NR²⁵R²⁵,

C₂₋₆ alkanoyl substituted by C₁₋₄ alkyl, aryl, C₁₋₄ alkoxy, CH₂(aryl¹), C₁₋₄ haloalkyl, halogen, OH, CN or NR²⁵R²⁶,

C₂₋₆ alkanoyloxy substituted by C₁₋₆ alkyl, aryl, C₁₋₄ alkoxy, CH₂(aryl¹), C₁₋₄ haloalkyl, halogen, OH, CN or NR²⁵R²⁶,

cycloalkyl¹oxy,

COcycloalkyl¹,

heterocycle substituted by one or more substituent selected from C₁₋₆ alkyl(substituted by OH), CONR²⁵R²⁶, CH₂CONR²⁵R²⁶, NR²⁵R²⁶, NHCONR²⁵R²⁶ alkyl), SO₂NR²⁵R²⁶, SO₂(C₁₋₆ alkyl), CO₂(C₁₋₆ alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl,

heterocyclyloxy substituted by one or more substituent selected from C₁₋₆ alkyl(substituted by OH), CONR²⁵R²⁶, CH₂CONR²⁵R²⁶, NR²⁵R²⁶ NHCONR²⁵R²⁶, CO(C₁₋₆ alkyl), SO₂NR²⁵R²⁶, SO₂(C₁₋₆ alkyl), CO₂(C₁₋₆ alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl,

WHEREIN aryl¹ is phenyl optionally fused to a C₅₇ carbocyclic ring, which group is optionally substituted by one or more substituent selected from C₁₋₆ alkyl(optionally substituted by OH, CN or halogen), C₁₋₆ haloalkoxy, OH, ═O, NY²WY¹, halogen, C₁₋₆ alkoxy, CONR²⁵R²⁶ CH₂CONR²⁵R²⁶, NR²⁵R²⁶, NHCONR²⁵R²⁶, CO(C₁₋₆ alkyl), COaryl, COheteroaryl, SO₂NR²⁵R²⁶, S(O)ₙ(C₁₋₄ alkyl), S(O)ₙ(aryl), S(O)ₙ(heteroaryl), CO₂(C₁₋₆ alkyl), CO₂(aryl), CO₂(heteroaryl), CO₂H, (CH₂)₁₋₄CO₂(C₁alkyl), (CH₂)₁₋₄CO₂H, (CH₂)₁₋₄CO₂(aryl), (CH₂)₁₋₄ CO₂(heteroaryl), O(CH₂)₁₋₄CO₂(C₁alkyl), O(CH₂)₁₋₄CO₂H, O(CH₂)₁₋₄CO₂(aryl), O(CH₂)₁₋₄ CO₂(heteroaryl), aryl, heterocyclyl, aryloxy, aryl(CH₂)oxy, aryl(CH₂), CN, O(CH₂)₁₋₄ CONR²⁵R²⁶ and C₃₋₇ cycloalkyl,

aryl² is phenyl optionally fused to a C₅₋₇ carbocyclic ring, which group is substituted by one or more substituent selected from C₁₋₆ alkyl(substituted by OH), CONR²⁵R²⁶, CH₂CONR²⁵R²⁶, NHCONR²⁵R²⁶, CO(C₁₋₆ alkyl), COaryl, COheteroaryl, SO₂NR²⁵R²⁶, S(O)ₙ(C₁alkyl), S(O).(aryl), S(O),(heteroaryl), CO₂(C₁₋₆ alkyl), CO₂(aryl), CO₂(heteroaryl), CO₂H, (CH₂)₁₋₄CO₂(C₁₋₆ alkyl), (CH₂)₁₋₄CO₂H, (CH₂)₁₋₄CO₂(aryl), (CH₂)₁₋₄CO₂(heteroaryl), O(CH₂)₁₋₄CO₂(C₁₋₆ alkyl), O(CH₂)₁₋₄CO₂H, O(CH₂)₁₋₄CO₂(aryl), O(CH₂)₁₋₄CO₂(heteroaryl), aryl, heterocyclyl, aryloxy, aryl(CH₂)oxy, aryl(CH₂), CN, O(CH₂)₁₋₄CONR²⁵R²⁶ and C₃₋₇ cycloalkyl,

heteroaryl¹ is heteroaryl optionally fused to a C₅₋₇ carbocyclic ring, which group is optionally substituted by one or more substituent selected from C₁₋₆ alkyl(optionally substituted by OH, CN or halogen), C₁₋₄ haloalkoxy, OH, ═O, NY²WY¹, halogen, C₁₋₆ alkoxy, CONR²⁵R²⁶, CH₂CONR²⁵R²⁶, NR²⁵R²⁶, NHCONR²⁵R²⁶, CO(C₁₋₆ alkyl), COaryl, Coheteroaryl, SO₂NR²⁵R²⁶, S(O)ₙ(C₁₋₆ alkyl), S(O)ₙ(aryl), S(O)ₙ(heteroaryl), CO₂(C₁₋₆ alkyl), CO₂(aryl), CO₂(heteroaryl), CO₂H, (CH₂)₁₋₄CO₂(C₁₋₆ alkyl), (CH₂)₁₋₄CO₂H, (CH₂)₁₋₄CO₂(aryl), (CH₂)₁₋₄CO₂(heteroaryl), O(CH₂)₁₋₄CO₂(C₁alkyl), O(CH₂)₁₋₄CO₂H, O(CH₂)₁₋₄CO₂(aryl), O(CH₂)₁₋₄CO₂(heteroaryl), aryl, heterocyclyl, aryloxy, aryl(CH₂)oxy, aryl(CH₂), CN, O(CH₂)₁₄CONR²⁵R²⁶ and C₃₋₇ cycloalkyl,

cycloalkyl¹ is a C₃₋₁₀ carbocyclic system with one or two rings and which is substituted by C₁₋₆ alkyl, aryl, C₁₋₆ alkoxy, CH₂(aryl¹), C₁₋₄ haloalkyl, halogen, OH, CN or NR²⁵R²⁶,

WITH THE PROVISO THAT THERE ARE NO N-R4 GROUPS WHEREIN THERE IS A HETERO-ATOM LINKED TO ANOTHER HETEROATOM VIA ONE SP3 CARBON

Z is a direct bond, CO or S(O)ₙ group,

B is (CH₂)ₚ,

R¹² and R¹³ each independently represent H or C₁₋₄ alkyl,

or R^{12 and R}¹³ can be taken together with the N atom to which they are attached to form a 4- to 7-membered heterocycle optionally comprising a further hetero moiety selected from NR¹⁶, O and/or S, and which is optionally substituted by one or more C₁₋₄ alkyl,

R¹⁴ and R¹⁵ each independently represent H, C₁₋₁₀ alkyl, C₃₋₁₀ alkenyl, C₃₋₁₀ alkynyl, C₃₋₈ cycloalkyl, aryl or heteroaryl,

or R¹⁴ and R¹⁵ can be taken together with the N atom to which they are attached to form a 4- to 7-membered heterocycle optionally comprising a further hetero moiety selected from NR¹⁶, O and/or S, and which is optionally substituted by one or more C₁₋₄ alkyl,

R¹⁶ is H, C₁₋₆ alkyl, C₃₋₆ cycloalkyl, (C₁₋₄ alkylene)(C₃₋₆ cycloalkyl) or (C₁₋₆ alkylene)aryl,

R⁵ and R⁸ when taken separately are each independently H, C₁₋₆ alkyl,

R⁵ and R⁸ can be taken together with the carbon atoms to which they are joined to form a C₃₋₈ cycloalkyl ring,

R⁶, R⁷, R⁹ and R¹⁰ when taken separately are H,

R⁵ and R⁶ or R⁷ can be taken together with the carbon atoms to which they are joined to form a C₃₋₈ cycloalkyl ring,

X is halogen, C₁₋₄ alkyl, C₁₋₄ alkoxy, C₁₋₄ haloalkyl or C₁₋₄ haloalkoxy,

m is 1 or 2;

n is 0, 1 or 2;

p is 0, 1, 2, 3, 4, 5, 6, 7, 8, 9 or 10;

q is 0 or 1;

“Naturally occuring amino acid substituent” means the α-substituent that occurs in any one of the following natural amino acids, glycine, alanine, valine, leucine, isoleucine, phenylalanine, tryptophan, tyrosine, histidine, serine, threonine, methionine, cysteine, aspartic acid, glutamic acid, asparagine, glutamine, lysine, arginine or proline;

“Heteroaryl” represents an aromatic ring containing up to four heteroatoms independently selected from N, O and S, and if a S atom is present in the ring, it can be present as part of a —S—, S(O)— or —S(O)₂— group, and which may be joined to the remainder of the compound via any available atom(s);

“Heterocycle” is a group containing 1, 2 or 3 rings, and which contains up to 4 ring heteroatoms selected from N, O and S and up to 18 ring carbon atoms;

“Aryl”, including in the definitions of “aryloxy”, etc., means a group comprising a phenyl ring and which may incorporate a further carbocyclic ring fused to said phenyl ring and which may be joined to the remainder of the compound via any available atom(s) (examples of such groups include naphthyl, indanyl, etc.);

“Alkyl”, “alkenyl” and “alkynyl” groups can be linear or branched if the number of carbon atoms allows;

“Cycloalkyl” groups can be polycyclic if the number of carbon atoms allows;

or a pharmaceutically or veterinarily acceptable derivative or prodrug thereof.

Where a fused heterocyclic group is present it can be attached to the remainder of the compound via any available atom(s).

“Haloalkyl”, “haloalkoxy” groups and the like can contain more than one halogen atom, and for instance can be per-halogenated.

Certain of the compounds of the invention can exist in one or more geometric and/or stereoisomeric forms. The present invention includes all such individual isomers and salts and prodrugs thereof.

Certain compounds of the present invention may exist in more than one tautomeric form. Similarly certain compounds of the invention may have zwitterionic forms. It is to be understood that the invention embraces all such tautomers, zwitterions and their derivatives.

The pharmaceutically acceptable salts of the compounds of the formula (I) include the acid addition and the base salts thereof. Suitable acid addition salts are formed from acids which form non-toxic salts and examples are the hydrochloride, hydrobromide, hydroiodide, sulphate, hydrogen sulphate, nitrate, phosphate, hydrogen phosphate, acetate, maleate, fumarate, lactate, tartrate, citrate, gluconate, succinate, benzoate, methanesulphonate, benzenesulphonate and p-toluenesulphonate salts. Suitable base salts are formed from bases which form non-toxic salts and examples are the aluminium, calcium, lithium, magnesium, potassium, sodium, zinc and diethanolamine salts. For a review on suitable salts see Berge et al, J. Pharm. Sci., 66, 1-19 (1977).

It will be appreciated by those skilled in the art that certain protected derivatives of compounds of formula (I), which may be made prior to a final deprotection stage, may not possess pharmacological activity as such, but may, in certain instances, be transformed after administration into or onto the body, for example by metabolism, to form compounds of formula (I) which are pharmacologically active. Such derivatives are included in the term “prodrug”. It will further be appreciated by those skilled in the art that certain moieties known to those skilled in the art as “pro-moieties”, for example as described in “Design of Prodrugs” by H Bundgaard (Elsevier) 1985, may be placed on appropriate functionalities when such functionalities are present in compounds of formula (I), also to form a “prodrug”. Further, certain compounds of formula I may act as prodrugs of other compounds of formula I. All protected derivatives, and prodrugs, of the compounds of formula I are included within the scope of the invention.

Preferably the “Ar” ring represents phenyl or pyridyl.

Most preferably the “Ar” ring represents a group of formula:

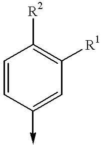

Preferably R¹ when taken alone is OH, CN, halogen, NO₂, NH₂, NY²WY¹ or Het¹.

More preferably R¹ when taken alone is OH, CN, I, C₁₋₆ , NH₂, NO₂, optionally benzo-fused heteroaryl, NHSO₂Y¹, NHCOY¹ or NHCO₂Y¹.

Yet more preferably R¹ when taken alone is OH, CN, I, Cl, NH₂, NO₂,1,2,3-triazolyl, 1,2,4-triazolyl, imidazol-2-yl, pyridin-2-yl, thien-2-yl, imidazol-4-yl, benzimidazol-2-yl, NHSO₂(C₁₋₆ alkyl), NHSO₂(C₁₋₆ alkyl substituted by methoxy, CONH₂, OH, CO₂(C₂₋₆ alkyl), phthalimido, NH₂ or halogen), NHSO₂NH₂, NHSO₂NH(C₁₋₆ alkyl), NHSO₂N(C₁₋₆ alkyl)₂, NHSO₂Het^{1a}, NHCO(C₁₋₆alkyl) or NHCO₂(C₁₋₆ alkyl).

Even more preferably R¹ is OH, NHSO₂CH₃, NHSO₂C₂H₅, NHSO₂(n-C₃H₇), NHSO₂(i-C₃H₇), NHSO₂(n-C₄H₇), NHSO₂NH(i-C₃H₇), NHSO₂(N-methylimidazol-4yl), NHSO₂(CH₂)₂OCH₃, NHSO₂(CH₂)₂OH, 1,2,4-triazolyl or imidazol-2-yl.

Most preferably R¹ is OH, NHSO₂CH₃, NHSO₂C₂H₅ or imidazol-2-yl.

Preferably R² when taken alone is H.

R¹ and R² when taken together with the carbon atoms to which they are attached are preferably an optionally benzo-fused 5- to 7-membered heteroaryl ring optionally substituted by C₁₋₄ alkyl or C₄ haloalkyl.

More preferably R¹ and R² when taken together with the carbon atoms to which they are attached are a 5-membered heteroaryl moiety optionally substituted by C₁₋₄ alkyl or C₁₋₄ haloalkyl.

Yet more preferably R¹ and R² when taken together with the carbon atoms to which they are attached are an imidazole group optionally 2-substituted by CF₃.

Preferably X is Cl.

Preferably n is 0.

Preferably q is 0.

Preferably R³ is H, CN, C₁₋₄ alkyl (optionally substituted by one or more halogen, OH, C₁₋₆ alkoxy, C₁₋₄ alkoxycarbonyl, C₂₋₆ alkanoyl, C₂₋₆ alkanoyloxy, C₂₋₆ alkyloxycarbonyloxy, NR¹²R¹³, CONR¹²R¹³ and/or NY²WY¹).

More preferably R³ is H, CH₃, C₂H₅, i-C₃H₇, n-C₃H₇ or CH₂OCH₃.

Most preferably R³ is CH₃.

Preferably R⁴ is C₁₋₁₀ alkyl substituted by one or more substituents selected from:

C₂₋₆ alkoxy [substituted by one or more groups selected from OH, NR²⁵R²⁶ CONR²⁵R²⁶, halogen, C₁ alkoxy, C₂₋₄ alkynyl, C₂4 alkenyl, heteroaryl¹, aryl¹, COCH₂CN, CO(heteroaryl¹), CO(aryl), CO₂(heteroaryl¹), COCH₂(aryl¹), COCH₂(heteroaryl¹), CO₂CH₂(aryl¹), CO₂CH₂(heteroaryl¹), S(O),(C₁₋₆ alkyl), S(O)ₙ(aryl¹), S(O),(heteroaryl¹), SO₂NR²⁵R²⁶ and cycloalkyl¹],

S(O)ₙC₁₋₆ alkyl [optionally substituted by one or more groups selected from OH, NR²⁵R²⁶, CONR²⁵R²⁶, halogen, C₁₋₄ alkoxy, C₂₄ alkynyl, C₂₋₄ alkenyl, heteroaryl¹, aryl, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O)n(C₁₋₆ alkyl), S(O),(aryl¹), S(O)n(heteroaryl¹),

SO₂NR²⁵R²⁶ and cycloalkyl¹],

aryl²,

CO₂CH₂(heteroaryl¹),

CO₂CH₂(aryl¹),

cycloalkyl¹,

CO(heteroaryl¹),

CO(aryl¹),

OCO(aryl¹),

OCO(heteroaryl¹),

OCO(C₁₋₆ alkyl),

OCOCH₂CN,

CO₂(heteroaryl¹),

CO₂(aryl¹),

COCH₂(heteroaryl¹),

S(O)ₙaryl¹,

S(O)ₙCH₂aryl¹,

S(O)ₙ(heteroaryl¹),

S(O)ₙCH₂(heteroaryl¹),

NHSO₂aryl¹,

NHSO₂(C₁₋₆ alkyl),

NHSO₂(heteroaryl¹),

NHSO₂CH₂(heteroaryl¹),

NHSO₂CH₂(aryl¹),

NHCOaryl¹,

NHCO(C₁₋₆ alkyl),

NHCONHaryl¹,

NHCONH(C₁l alkyl),

NHCOheteroaryl¹,

NHCONHheteroaryl¹,

NHCO₂(aryl¹),

NHCO₂(C₁₋₆ alkyl),

NHCO₂(heteroaryl¹),

aryl oxy

heteroaryl¹oxy,

C₁₋₆ alkoxycarbonyl substituted by C₁₋₆ alkyl, aryl, C₁-₆ alkoxy, CH₂(aryl¹), C₁₋₄ haloalkyl, halogen, OH, CN or NR²⁵R²⁶,

C₂₋₆ alkanoyl substituted by C₁₋₆ alkyl, aryl, C₁₋₆ alkoxy, CH₂(aryl¹), C₁₋₄ haloalkyl, halogen, OH, CN or NR²⁵R²⁶,

C₂₋₆ alkanoyloxy substituted by C₁₋₄ alkyl, aryl, C₁₋₆ alkoxy, CH₂(aryl¹), C₁₋₄ haloalkyl, halogen, OH, CN or NR²⁵R²⁶,

cycloalkyl¹oxy,

COcycloalkyl¹,

heterocycle substituted by one or more substituent selected from C₁₋₆ alkyl(substituted by OH), CONR²⁵R²⁶, CH₂CONR²⁵R²⁶ NR²⁵R², NHCONR²⁵R²⁶, CO(C₁₋₆ alkyl), SO₂NR²⁵R²⁶, SO₂(C₁₋₆ alkyl), CO₂(Cl alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl,

heterocyclyloxy substituted by one or more substituent selected from C₁₋₆ alkyl(substituted by OH), CONR²⁵R²⁶, CH₂CONR²⁵R²⁶, NR²⁵R²⁶, NHCONR²⁵R²⁶, CO(C₁₋₆ alkyl), SO₂NR²⁵R²⁶, SO₂(C₁₋₆ alkyl), CO₂(C₁₋₆ alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl,

More preferably R⁴ is C₁₋₁₀ alkyl substituted by cycloalkyl¹.

Yet more preferably R⁴ is C₂₋₄ alkyl substituted by cycloalkyl¹.

Further more preferably R⁴ is propyl substituted by cycloalkyl¹.

Further yet more preferably R⁴ is propyl substituted by a C₃₋₁₀ carbocyclic system with one or two rings and which is substituted by OH.

Even more preferably R⁴ is propyl substituted by (cyclohexyl substituted by OH) Most preferably R⁴ is (1-hydroxycyclohexyl)prop-3-yl.

Another preferred group of compounds are those wherein R⁴ takes the values as specified in the Examples 145-203 below.

Preferably R⁵, R⁶, R⁷, R⁸ R⁹ and R¹⁰ are each taken separately and are H.

A preferred group of substances are those in which the “Ar” ring, R¹, R², R³, R⁴, R⁵, R⁶, R⁷, R⁸, R⁹, R¹⁰, q and (X)ₙ have the values as detailed in the Examples below.

The invention further provides synthetic methods for the production of compounds and salts of the invention, which are described below and in the Examples and Preparations. The skilled man will appreciate that the compounds of the invention could be made by methods other than those herein described, by adaptation of the methods herein described and/or adaptation of methods known in the art, for example the art described herein, or using standard textbooks such as

“Comprehensive Organic Transformations—A Guide to Functional Group Transformations”, R C Larock, VCH (1989 or later editions),

“Advanced Organic Chemistry—Reactions, Mechanisms and Structure”, J.March, Wiley-lnterscience (3rd or later editions),

“Organic Synthesis—The Disconnection Approach”, S Warren (Wiley), (1982 or later editions),

“Designing Organic Syntheses” S Warren (Wiley) (1983 or later editions), “Guidebook To Organic Synthesis” R K Mackie and D M Smith (Longman) (1982 or later editions), etc.,and the references therein as a guide.

It is to be understood that the synthetic transformation methods mentioned herein are exemplary only and they may be carried out in various different sequences in order that the desired compounds can be efficiently assembled. The skilled chemist will exercise his judgement and skill as to the most efficient sequence of reactions for synthesis of a given target compound. For example, substituents may be added to and/or chemical transformations performed upon, different intermediates to those mentioned hereinafter in conjunction with a particular reaction. This will depend inter alia on factors such as the nature of other functional groups present in a particular substrate, the availability of key intermediates and the protecting group strategy (if any) to be adopted. Clearly, the type of chemistry involved will influence the choice of reagent that is used in the said synthetic steps, the need, and type, of protecting groups that are employed, and the sequence for accomplishing the synthesis. The procedures may be adapted as appropriate to the reactants, reagents and other reaction parameters in a manner that will be evident to the skilled person by reference to standard textbooks and to the examples provided hereinafter.

It will be apparent to those skilled in the art that sensitive functional groups may need to be protected and deprotected during synthesis of a compound of the invention. This may be achieved by conventional methods, for example as described in “Protective Groups in Organic Synthesis” by T W Greene and P G M Wuts, John Wiley & Sons Inc (1999), and refernces therein. Functional groups which may desirable to protect include oxo, hydroxy, amino and carboxylic acid. Suitable protecting groups for oxo include acetals, ketals (e.g. ethylene ketals) and dithianes. Suitable protecting groups for hydroxy include trialkylsilyl and diarylalkylsilyl groups (e.g. tert-butyldimethylsilyl, tert-butyldiphenylsilyl or trimethylsilyl) and tetrahydropyranyl. Suitable protecting groups for amino include tert-butyloxycarbonyl, 9-fluorenylmethoxycarbonyl or benzyloxycarbonyl. Suitable protecting groups for carboxylic acid include C₁₋₆ alkyl or benzyl esters.

In the Methods below, unless otherwise specified, the substituents are as defined above with reference to the compounds of formula (I).

The invention provides a process for the preparation of compounds of formula I as defined above, or a pharmacutically or veterinarily acceptable derivative thereof, which comprises:

(a) for compounds of formula I in which q is 0 and R¹ represents NY²WY¹, reacting a compound of formula II,

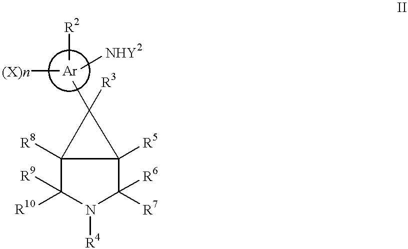

with a compound of formula III,

Z¹-WY¹ III

wherein Z¹ is a suitable leaving group, such as halogen or Y¹SO₂O—;

(b) for compounds of formula I in which q is 0 and R⁶ and R⁷ both represent H, reduction of a compound of formula IV,

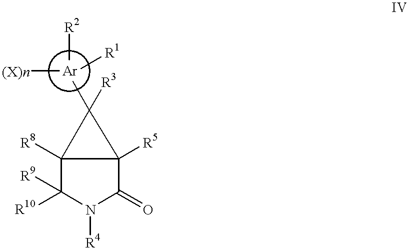

using a suitable reducing agent;

(c) for compounds of formula I in which q is 0 and R⁹ and R¹⁰ both represent H, reduction of a compound of formula V,

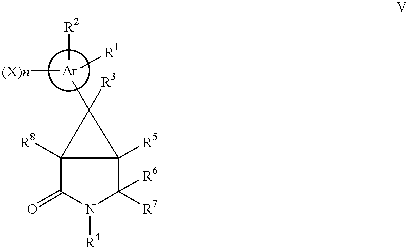

using a suitable reducing agent;

(d) for compounds of formula I in which q is 0 and R¹ and R² are attached to adjacent carbon atoms and are taken together with the carbon atoms to which they are attached to represent Het^{1a}, in which Het^{1a} represents an imidazolo unit, reaction of a corresponding compound of formula VI,

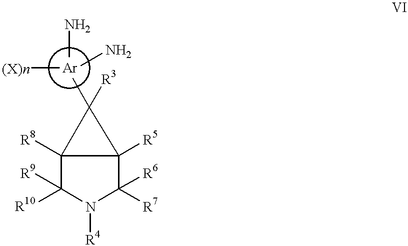

with a compound of formula VII,

R^{y}CO₂H VII

wherein R^{y} represents H or any of the optional substituents on Het^{1a} (as defined above), preferably H, C₁₋₄ alkyl or C₁₋₄ haloalkyl;

(e) where q is 0, reacting a compound of formula VIII,

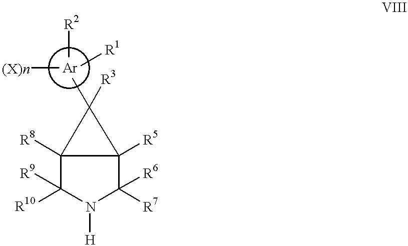

with a compound of formula IX,

R⁴-Lg IX

wherein Lg is a leaving group;

(f) for compounds of formula I in which q is 0 and R⁶, R⁷, R⁹ and R¹⁰ are all H, reduction of a compound of formula X,

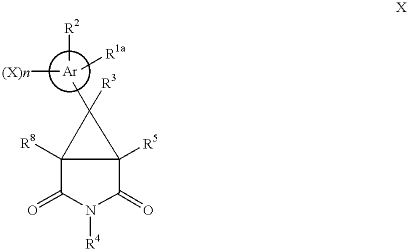

with a suitable reducing agent;

(g) for compounds of formula I in which q is 0 and R¹ represents OH, reacting a compound of formula II in which Y² is H, as defined above, with fluoroboric acid and isoamyl nitrite;

(h) for compounds of formula I in which q is 0 and R¹ represents Cl, reacting a compound of formula II in which y² is H, as defined above, with sodium nitrite in the presence of dilute acid, followed by reaction with copper (I) chloride in the presence of concentrated acid;

(i) for compounds of formula I in which q is 1, reacting a compound of formula I where q is 0 with a suitable oxidising agent such as aqueous hydrogen peroxide;

(j) for compounds of formula I where q is 0, by reduction of a corresponding compound of formula XXXI,

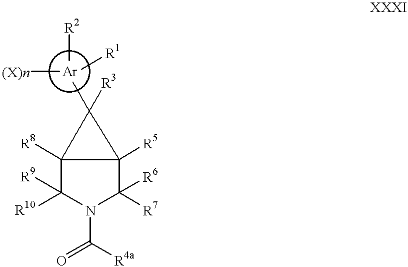

where R^{4a}C₂ takes the same meaning as R⁴ as defined above; or

(k) for compounds of formula (1) where q is 0, reductive amination reaction of the amine of formula VIII above with an aldehyde of formula R^{4a}-CHO wherein R^{4a}CH₂ takes the same meaning as R⁴ as defined above,

and where desired or necessary converting the resulting compound of formula I into a pharmaceutically or veterinarily acceptable derivative or vice versa.

In process (a), the reaction may be carried out at between 0° C. and room temperature in the presence of a suitable base (e.g. pyridine) and an appropriate organic solvent (e.g. dichloromethane).

Compounds of formula II may be prepared by reduction of a corresponding compound of formula XI or formula XII,

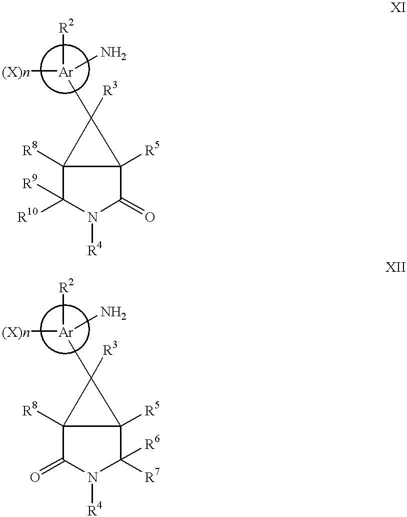

in the presence of a suitable reducing agent, such as lithium aluminium hydride. The reaction may be carried out at between room temperature and reflux temperature in the presence of a suitable solvent (e.g. tetrahydrofuran).

Compounds of formula Xl and XII may be prepared by reduction of the corresponding —NO₂ compounds under conditions that are well known to those skilled in the art (e.g. using H₂/Raney Ni or in the presence of CaCl₂ and iron powder, in the presence of a suitable solvent system (e.g. EtOH, EtOAc and/or water)). The skilled person will appreciate that, in preparing a compound of formula II, in which Y² is H, from such a corresponding —NO₂ compound, the two above-mentioned reduction steps may be performed in the same step or sequentially in any order.

The said corresponding -NO₂ compounds may be prepared by reaction of a compound of formula XII or formula XIV, as appropriate,

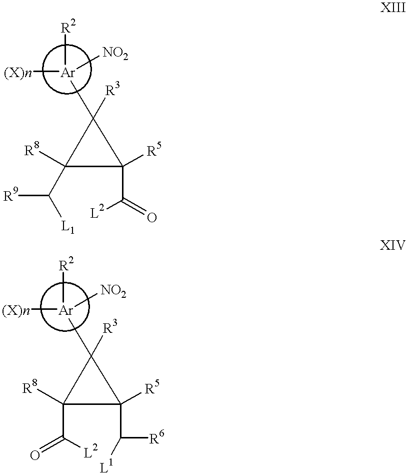

wherein L¹ represents a suitable leaving group [such as halo (e.g. chloro or bromo)], L² represents a suitable leaving group (such as C₁₋₃ alkoxy) and R³ is as defined above, with a compound of formula XV,

R⁴NH₂ XV

The reaction may be carried out at between room temperature and reflux temperature in the presence of a suitable base (e.g. NaHCO₃) and an appropriate organic solvent (e.g. dimethylformamide), or at a higher temperature (e.g. between 50 and 200° C., preferably between 100 and 160° C.) in the presence of neat compound of formula XV.

Compounds of formula XIII and XIV may be prepared in accordance with standard techniques. For example, compounds of formula XIII and XIV may be prepared by reacting a corresponding compound of formula XVI or XVII,

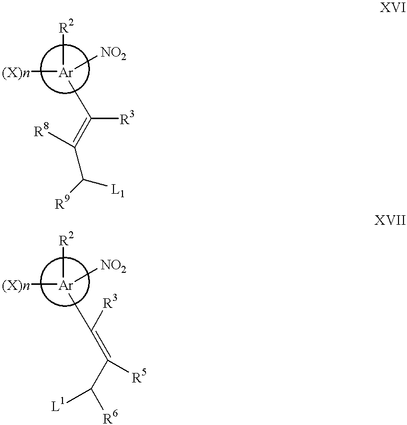

with a compound of formula XVIII or XIX respectively,

N₂CHR⁵COL² XVIII

N₂CHR⁸COL² XIX

wherein L² is as defined above. The reaction may be carried out at room temperature in the presence of a suitable catalyst [e.g. Rh₂(OAc)₄] and an appropriate non-protic organic solvent (e.g. dichloromethane).

Compounds of formula XVI and formula XVII are available or can be prepared using known techniques. Compounds of formula XVI and formula XVII may, for example, be prepared from corresponding compounds of formula XX,

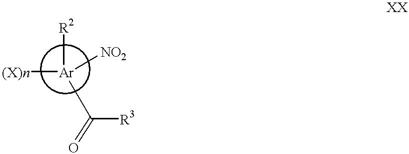

for example by performing a Wittig reaction using an appropriate provider of the nucleophilic group RO₂C—CR⁵H⁻ or RO₂C—CR⁸H⁻ (wherein R represents lower (e.g. C₁₋₃) alkyl), as appropriate, under conditions that are well known to those skilled in the art. The —CO₂R group of the resulting compound may be converted to an appropriate —CH₂L¹ group using standard techniques (e.g. reduction of the ester to the primary alcohol and conversion of the latter to an alkyl halide) under conditions that are well known to those skilled in the art.

In processes (b) and (c), suitable reducing agents include lithium aluminium hydride. The reaction may be carried out at between room temperature and reflux temperature in the presence of a suitable solvent (e.g. tetrahydrofuran).

Compounds of formula II may be prepared by reduction of the corresponding compound of formula XXX,

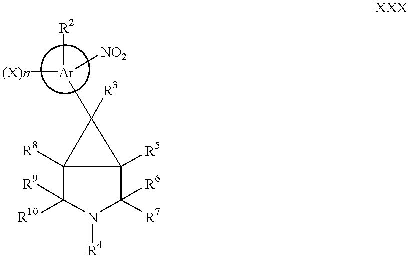

by analogy to the process steps mentioned above.

Compounds of formula IV and V may be prepared respectively from compounds of formula XXI and XXII,

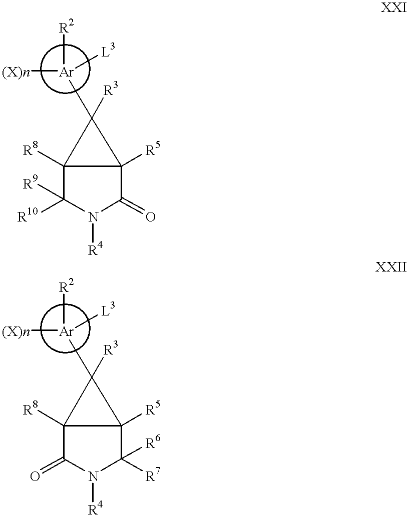

wherein L³ represents a group that is capable of undergoing functional group transformations (e.g. cyano) using standard functional group substitution or conversion techniques.

For example:

(1) Compounds of formula IV and V in which R¹ represents 1,2,4-triazol-3-yl may be prepared by reaction of an appropriate compound of formula XXI or XXII in which L³ represents —CN with HCl (gas) in the presence of an appropriate lower alkyl alcohol (e.g. ethanol), for example at between 0° C. and room temperature, followed by reaction of the resultant intermediate with formic acid hydrazide (e.g. at reflux temperature, with or without the presence of a suitable organic solvent (e.g. methanol), followed by, if necessary, removing the solvent and heating the resultant residue to a high temperature (e.g. about 150° C.)).

(2) Compounds of formula IV and V in which R¹ represents imidazol-2-yl may be prepared by reaction of an appropriate compound of formula XXI or XXII in which L³ represents —CN with HCl (gas) in the presence of an appropriate lower alkyl alcohol (e.g. ethanol), for example at between 0° C. and room temperature, followed by reaction of the resultant intermediate with aminoacetaldehyde dialkylacetal (e.g. dimethylacetal) (e.g. at or around reflux temperature in the presence of an appropriate solvent, such as methanol).

(3) Compounds of formula IV and V in which R¹ represents 1,2,3-triazol-5-yl may be prepared by reaction of an appropriate compound of formula XXI or XXII in which L³ represents —CN with diazomethane, or a protected (e.g. trialkylsilyl) derivative thereof, for example at between 0° C. and room temperature in the presence of a suitable base (e.g. n-BuLi) and, optionally, an appropriate organic solvent (e.g. THF), followed by removal of the protecting group as necessary.

(4) Compounds of formula IV and V in which R¹ represents benzimidazol-2-yl may be prepared by reaction of an appropriate compound of formula XXI or XXII in which L³ represents C═NH(OEt) with 1,2-diaminobenzene. The reaction may be carried out in a solvent such as methanol, at an elevated temperature (such as the reflux temperature of the solvent). Preparations 81, etc. provide further details.

Compounds of formula IV and V in which R¹ represents Het¹ may also be prepared from compounds of formula XI and XII respectively according to the following scheme:

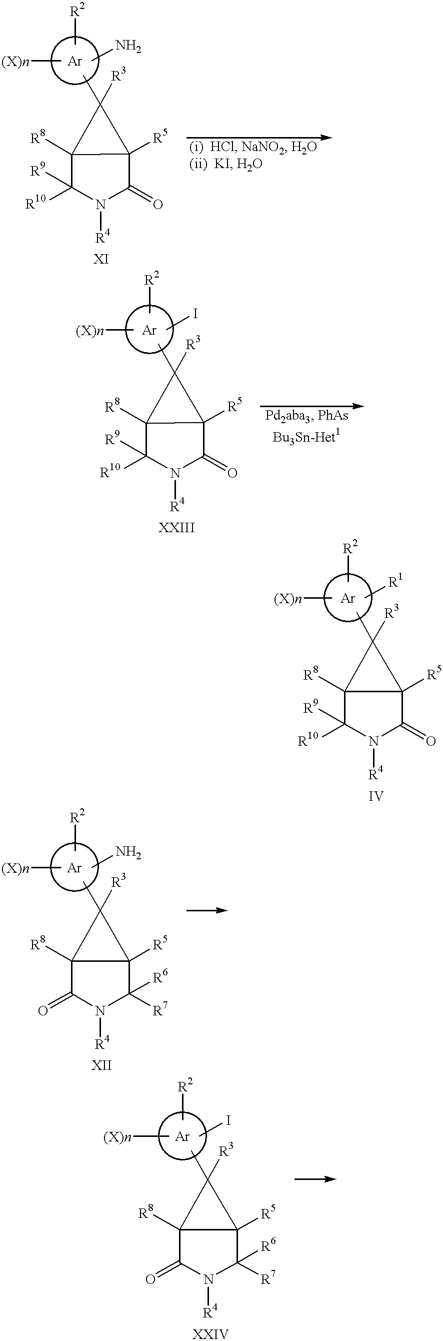

wherein Het¹ is defined above. Further details may be found in Preparations 67, 68, etc. in WO00/39089, herein incorporated by reference in its entirety.

Compounds of formula XXI and XXII may be prepared in analogous fashion to methods described herein, for example those described hereinbefore for preparation of compounds of formula II.

Other compounds of formula (IV) and (V) may be prepared by analogy with methods described herein (e.g. by analogy with methods described hereinbefore for preparation of compounds of formula XI and XII (and especially the corresponding —NO₂ compound)).

In process (d), the reaction may be carried out by heating under reflux, with or without the presence of an appropriate organic solvent.

Compounds of formula VI may be prepared using known techniques. For example, compounds of formula VI may be prepared by nitration (at the 4-position) of a corresponding 3-aminobenzene compound (a compound of formula II), which latter compound may be activated by converting the 3-amino group to a 3-amido group, followed by hydrolysis of the amide and reduction of the 4-nitrobenzene compound. All of these reactions may be performed using techniques that are familiar to the skilled person, and are illustrated in Preparations 45-48, etc. below.

In process (e), suitable leaving groups that Lg may represent include halogen, such as bromine, or a sulphonate group such as tosylate, mesylate or triflate. The reaction may be carried out in a polar solvent that does not adversely affect the reaction, at a suitable temperature, e.g. 0-150° C., in the presence of a base. A catalyst such as sodium iodide may optionally be added.

Preferable choices are a slight excess of R⁴-Lg, where Lg═Cl or Br, an excess of base (2.0-4.0 eq), such as K₂CO₃, NaHCO₃, or a tertiary amine, such as triethylamine or Hunigs base, in a polar solvent, such as THF, DMF, or MeCN, at between 40 and 120° C., optionally in the presence of a catalyst such as Nal or Kl, for 2-24 hr.

see R C Larrock in “Comprehensive Organic Transformations—A Guide to Functional Group Preparations”, VCH, (1989), p 397, and references cited therein.

Compounds of formula VIII may be prepared from compounds of formula XXV,

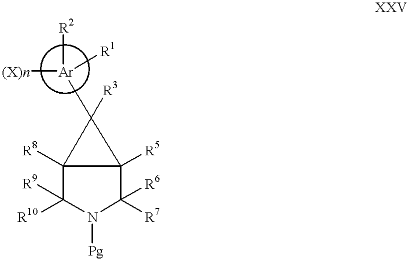

wherein Pg represents a suitable protecting group. Suitable protecting groups include allyl, which may be removed using a palladium (0) catalyst and N,N-dimethylbarbituric acid (see Preparation 53, etc. below). Compounds of formula XXV may be prepared using analogous methods to those described herein for the preparation of compounds of formula I.

In process (f), suitable reducing agents include lithium aluminium hydride. The reaction may be carried out in a solvent that does not adversely affect the reaction (for example tetrahydrofuran), at an elevated temperature (for example the reflux temperature of the solvent).

Compounds of formula X may be prepared by reacting a compound of formula XXVI with a compound of formula XXVII in the presence of an oxidizing agent. Suitable oxidizing agents include manganese dioxide. The reaction may be carried out in a solvent that does not adversely affect the reaction (for example dioxan), at an elevated temperature such as the reflux temperature of the solvent (for example see Preparation 77, WO00/39089). The intermediate compounds XXIXa are isolatable using suitable conditions (e.g. see Preparation 58, WO00/39089).

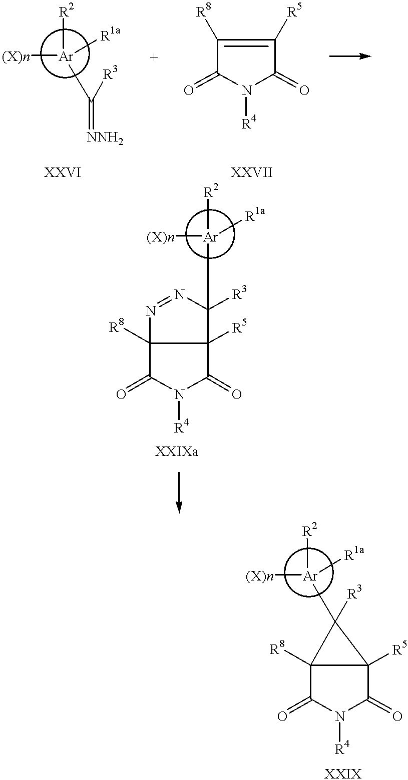

Compounds of formula XXVI may be prepared from compounds of formula XXVIII, by reaction of the corresponding ketone with hydrazine monohydrate using known techniques (and as described in Preparation 76, etc. WO00/39089).

Process (f) is particularly useful when Ar represents an optionally benzo-fused 5- or 6-membered heteroaryl ring. A similar methodology may be used to obtain compounds of formula II: the precursor nitro compound may be prepared from a compound of formula XX, as defined above, using the steps described above (see for example Preparations 57-61, WO00/39089).

In process (g), the reaction may be carried out in a solvent that does not adversely affect the reaction (for example ethanol), first below room temperature and then at an elevated temperature (Examples 79, etc. WO00/39089, provides further details).

In process (h), suitable acids include dilute aqueous hydrochloric acid and concentrated hydrochloric acid, respectively. The reaction may be carried out at or around room temperature, finishing at an elevated temperature (for example 90° C.). Example 51 WO00/39089 provides further details.

In process (j), the compound of formula XXXI may be prepared by acylation of the compound of formula VIII as defined above, with an acylating agent of the formula R^{4a}CO-Lg, where Lg is a suitable leaving group as defined above with respect to (e), and includes halogen, (alkyl, haloalkyl or aryl)sulphonate, OCOR^{4a} (i.e. an acid anhydride) and the like, well known to those practising in the art. See for example the conditions used for Preparation 47. The coupling can optionally be carried out in the presence of a catalyst, for example DMAP, in a suitable solvent; see R C Larrock in “Comprehensive Organic Transformations—A Guide to Functional Group Preparations”, second edition, (1999), pp 1941-1949, and references cited therein. Preferably the carboxylic acid (0.9-1.1 eq), 1-(3-dimethylaminopropyl)-3-ethylcarbodiimide. HCl (1-1.5 eq) and 1-hydroxybenzotriazole (1.0 eq) are stirred in DMF or DCM at RT for 5-15 min and then the amine salt (1 eq) and base (NaHCO3 or organic base, Et3N or Hunigs base (2-4 eq)) are added, the reaction taking 2-24 hr at RT.

The amide bond can be reduced with a suitable reducing agent, for example lithium aluminium hydride or borane, in an ethereal solvent, such as THF, at 0-100° C. to generate the desired tertiary amine, see R C Larrock in “Comprehensive Organic Transformations—A Guide to Functional Group Preparations”, VCH, (1989), pp 432-434, and references cited therein. Preferably the amide (1.0 eq) is treated with lithium aluminium hydride (1.0-3 eq), at 0° C.-RT, in THF, for 1-24 hr.

In process (k) the appropriate aldehyde is reacted with an amine, optionally present as an acid addition salt, in the presence of a suitable reducing agent (such as sodium cyanoborohydride, sodium triacetoxyborohydride, or catalytic hydrogenation with Pd, Pt or Ni catalysts). The reaction is suitably performed in the presence of acetic acid at 0-100° C. in THF, methanol, DCM (dichloromethane), or DCE (1,2-dichloroethane), for a suitable time such as 1-24 hr. Preferably the amine salt, such as the trifluoroacetic acid (TFA) salt, is treated with an organic base (1-3 mole equivalents), such as triethylamine or Hunigs base, and then the aldehyde (1-1.5 mole equivalents), followed by sodium triacetoxyborohydride (1-2.0 mole equivalents), in DCM or DCE, at room temperature for 2-24 hr. see R C Larrock; “Comprehensive Organic Transformations—A Guide to Functional Group Preparations”, second edition, (1999), p 835-842, and references cited therein, and Abdel-Magid et al, J. Org. Chem., 1996, 61, 3849.

The aldehydes used in this process may be prepared from the corresponding alcohols using suitable oxidising agents; see R C Larrock in “Comprehensive Organic Transformations—A Guide to Functional Group Preparations”, second edition, (1999), pp 1234-1236 and 1238-1247, and references cited therein. Preferred oxidants are tetrapropylammonium perruthenate (Ley, et.al., Synthesis, 1994, 639-666), Swern oxidation and related methods (Tidwell, Organic Reactions, 1990, 39, 297-572), and Dess-Martin Periodinane reagent (Dess et al., J. Org. Chem., 1983, 48, 4155-4156).

Various functional group interconversions on compounds of formula (I), or intermediates thereto, may be carried out to give different compounds of formula (I) or intermediates. Some of these are mentioned below.

Anilines can be converted to a urea using potassium cyanate (excess) in an acidic aqueous solution, see Cross et al., J. Med. Chem., 1985, 28,1427-1432.

Esters can be converted to the corresponding alcohols using a suitable reducing agent, see Larock, Comprehensive Organic Transformations—A Guide to Functional Group Preparations, second edition, (1999), pp 1117-1120 and references cited therein. Suitable reducing agents include diisobutylaluminium hydride (DIBAL, see Winterfeldt, Synthesis, 1975, 617) and lithium aluminium hydride (LiAIH4, see Brown, Org. Reactions, 1951, 6, 469) -viz. reaction of the type:

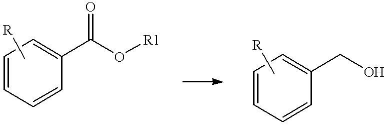

Alcohols can be prepared from a corresponding acid using a suitable reducing agent; see Larock, Comprehensive Organic Transformations—A Guide to Functional Group Preparations, second edition, (1999), pp 1114-1116. Preferably the reducing agent is either borane (BH3 (1-2 eq), J. Org. Chem., 1973, 38, 2786), or LiAIH4 (1-4 eq), in an ethereal solvent, such as THF, 0-80° C., for 1-24 hr. - viz. reaction of the type:

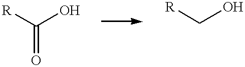

Direct methods to prepare alkyl halides and alyl sulphonates from their alcohols are described by R C Larrock, Comprehensive Organic Transformations—A Guide to Functional Group Preparations, second edition, (1999),

pp 689-700, and references cited therein.

Benzylacetals can be treated with a suitable reducing agent in the presence of a Lewis acid or organic acid to give benyloxyalcohols.

For representative examples see Organic Preparations and Procedures, Int., 1991, 23, 4, 427-431, ZrCl4/LiAlH4; J. Org. Chem., 1987, 52, 2594, Zn(BH4)2/Me3SiCl; and Organic Preparations and Procedures, Int., 1985, 17(1), 11-16, NaBH4/TFA.

viz. reaction of the type:

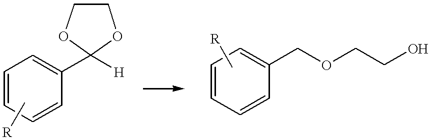

It will be apparent to those skilled in the art that compounds of formula I may be converted to other compounds of formula I using known techniques. For example, compounds of formula I in which Y¹ represents alkoxycarbonyl may be converted to compounds in which Y¹ represents alkyl substituted by OH, by reduction using LiAIH₄ (Example 57 provides further details). Similarly, intermediate compounds may be interconverted using known techniques (see for example Preparation 85).

The intermediate compounds such as those of formulae III, XV, XVIII, XIX, XX, VII, IX, XXVI, XXVII and XXVIII, and derivatives thereof, when not commercially available or not subsequently described, may be obtained either by analogy with the processes described herein, or by conventional synthetic procedures, in accordance with standard techniques, from readily available starting materials using appropriate reagents and reaction conditions.

The invention further provides the intermediate compounds of formulae II, IV, V, VI, X, X^{a}, Xl, XII, XXI, XXII, XXIII, XXIV, XXIX, XXIXa, XXX, and XXXI as defined above.

Where desired or necessary, the compound of formula (I) can be converted into a pharmaceutically acceptable salt thereof, conveniently by mixing together solutions of a compound of formula (I) and the desired acid or base, as appropriate. The salt may be precipitated from solution and collected by filtration, or may be collected by other means such as by evaporation of the solvent. Both types of salt may also be formed or interconverted using ion-exchange resin techniques.

The compounds of the invention may be purified by conventional methods, for example separation of diastereomers may be achieved by conventional techniques, e.g. by fractional crystallisation, chromatography or H.P.L.C. of a stereoisomeric mixture of a compound of formula (I) or a salt thereof. An individual enantiomer of a compound of formula (I) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active base or acid.

The compounds of the invention are useful because they possess pharmacological activity in animals, especially mammals including humans. They are therefore indicated as pharmaceuticals and, in particular, for use as animal medicaments.

According to a further aspect of the invention there is provided the compounds of the invention for use as medicaments, such as pharmaceuticals and animal medicaments, such as for the treatment of opiate-mediated diseases and conditions.

By the term “treatment”, this term includes both therapeutic (curative) and prophylactic treatment.

In particular, the substances of the invention have been found to be useful in the treatment of diseases and conditions modulated via opiate receptors, such as irritable bowel syndrome; constipation; nausea; vomiting; pruritus; eating disorders; opiate overdoses; depression; smoking and alcohol addiction; sexual dysfunction; shock; stroke; spinal damage and/or head trauma; and conditions characterised by having pruritis as a symptom.

Thus, according to a further aspect of the invention there is provided the use of the compounds of the invention in the manufacture of a medicament for the treatment of a disease modulated via an opiate receptor. There is further provided the use of the compounds of the invention in the manufacture of a medicament for the treatment of as irritable bowel syndrome; constipation; nausea; vomiting; pruritus; eating disorders; opiate overdoses; depression; smoking and alcohol addiction; sexual dysfunction; shock; stroke; spinal damage and/or head trauma; and conditions characterised by having pruritis as a symptom.

The compounds of the invention are thus expected to be useful for the curative or prophylactic treatment of pruritic dermatoses including allergic dermatitis and atopy in animals and humans. Other diseases and conditions which may be mentioned include contact dermatitis, psoriasis, eczema and insect bites.

Thus, the invention provides a method of treating or preventing a disease modulated via an opiate receptor. There is further provided a method of treating irritable bowel syndrome; constipation; nausea; vomiting; pruritus; eating disorders; opiate overdoses; depression; smoking and alcohol addiction; sexual dysfunction; shock; stroke; spinal damage and/or head trauma; or a medical condition characterised by pruritus as a symptom in an animal (e.g. a mammal), which comprises administering a therapeutically effective amount of a compound of the invention to an animal in need of such treatment.

The compounds of the invention will normally be administered orally or by any parenteral route, in the form of pharmaceutical preparations comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses (see below).

While it is possible to administer a compound of the invention directly without any formulation, the compounds are preferably employed in the form of a pharmaceutical, or veterinary, formulation comprising a pharmaceutically, or veterinarily, acceptable carrier, diluent or excipient and a compound of the invention. The carrier, diluent or excipient may be selected with due regard to the intended route of administration and standard pharmaceutical, and/or veterinary, practice. Pharmaceutical compositions comprising the compounds of the invention may contain from 0.1 percent by weight to 90.0 percent by weight of the active ingredient.

The methods by which the compounds may be administered for veterinary use include oral administration by capsule, bolus, tablet or drench, topical administration as an ointment, a pour-on, spot-on, dip, spray, mousse, shampoo, collar or powder formulation or, alternatively, they can be administered by injection (eg subcutaneously, intramuscularly or intravenously), or as an implant. Such formulations may be prepared in a conventional manner in accordance with standard veterinary practice.

The formulations will vary with regard to the weight of active compound contained therein, depending on the species of animal to be treated, the severity and type of infection and the body weight of the animal. For parenteral, topical and oral administration, typical dose ranges of the active ingredient are 0.01 to 100 mg per kg of body weight of the animal. Preferably the range is 0.1 to 10 mg per kg.

In any event, the veterinary practitioner, or the skilled person, will be able to determine the actual dosage which will be most suitable for an individual patient, which may vary with the species, age, weight and response of the particular patient. The above dosages are exemplary of the average case; there can, of course, be individual instances where higher or lower dosage ranges are merited, and such are within the scope of this invention.

For veterinary use, the compounds of the invention are of particular value for treating pruritus in domestic animals such as cats and dogs and in horses.

As an alternative for treating animals, the compounds may be administered with the animal feedstuff and for this purpose a concentrated feed additive or premix may be prepared for mixing with the normal animal feed.

For human use, the compounds are administered as a pharmaceutical formulation containing the active ingredient together with a pharmaceutically acceptable diluent or carrier. Such compositions include conventional tablet, capsule and ointment preparations which are formulated in accordance with standard pharmaceutical practice.

Compounds of the invention may be administered either alone or in combination with one or more agents used in the treatment or prophylaxis of disease or in the reduction or suppression of symptoms. Examples of such agents (which are provided by way of illustration and should not be construed as limiting) include antiparasitics, eg fipronil, lufenuron, imidacloprid, avermectins (eg abamectin, ivermectin, doramectin), milbemycins, organophosphates, pyrethroids; antihistamines, eg chlorpheniramine, trimeprazine, diphenhydramine, doxylamine; antifungals, eg fluconazole, ketoconazole, itraconazole, griseofulvin, amphotericin B; antibacterials, eg enroflaxacin, marbofloxacin, ampicillin, amoxycillin; anti-inflammatories eg prednisolone, betamethasone, dexamethasone, carprofen, ketoprofen; dietary supplements, eg gamma-linoleic acid; and emollients. Therefore, the invention further provides a product containing a compound of the invention and one or more selected compounds from the above list as a combined preparation for simultaneous, separate or sequential use in the treatment of diseases modulated via opiate receptors The skilled person will also appreciate that compounds of the invention may be taken as a single dose or on an “as required” basis (i.e. as needed or desired).

Thus, according to a further aspect of the invention there is provided a pharmaceutical, or veterinary, formulation including a compound of the invention in admixture with a pharmaceutically, or veterinarily, acceptable adjuvant, diluent or carrier.

Compounds of the invention may also have the advantage that, in the treatment of human and/or animal patients, they may be more efficacious than, be less toxic than, have a broader range of activity than, be more potent than, produce fewer side effects than, be more easily absorbed than, or they may have other useful pharmacological properties over, compounds known in the prior art.

The biological activities of the compounds of the present invention were determined by the following test method.

Biological Test

Compounds of the present invention have been found to display activity in three opioid receptor binding assays selective for the mu, kappa and delta opioid receptors in dog brain. The assays were conducted by the following procedure.

Laboratory bred beagles were used as a source of dog brain tissue. Animals were euthanaised, their brains removed and the cerebellum discarded. The remaining brain tissue was sectioned into small pieces approximately 3 g in weight and homogenised in 50 mM Tris pH 7.4 buffer at 4° C. using a Kinematica Polytron tissue homogeniser. The resulting homogenate was centrifuged at 48,400×g for 10 minutes and the supernatant discarded. The pellet was resuspended in Tris buffer and incubated at 37° C. for 10 minutes. Centrifugation, resuspension and incubation steps were repeated twice more, and the final pellet was resuspended in Tris buffer and stored at −80° C. Membrane material prepared in this manner could be stored for up to four weeks prior to use.

For mu, kappa and delta assays, increasing concentrations of experimental compound (5×10⁻¹² to 10⁻⁵M), Tris buffer and ³H ligand, (mu=[D-Ala²,N-Me-Phe⁴,Gly-ol⁵]-Enkephalin, DAMGO; kappa U-69,593; delta=Enkephalin, [D-pen^{2,5}] DPDPE), were combined in polystyrene tubes. The reaction was initiated by the addition of tissue, and the mixture was incubated at room temperature for 90 minutes. The reaction was terminated by rapid filtration using a Brandel Cell Harvester™ through BetaplateTm GF/A glass fibre filters pre-soaked in 50 mM Tris pH 7.4, 0.1% polyethylenimine buffer. The filters were then washed three times with 0.5 ml ice-cold Tris pH 7.4 buffer. For mu and delta assays, washed filters were placed in bags and Starscint™ scintillant added, for the kappa assay Meltilex™ B/HS solid scintillant was used. Bags containing the filters and scintillant were heat sealed and counted by a Betaplate™ 1204 beta counter.

Duplicate samples were run for each experimental compound and the data generated was analysed using IC₅₀ analysis software in Graphpad Prism. Ki values were calculated using Graphpad Prism according to the following formula:

Ki=IC₅₀/1+[³H ligand]/K_{D}

where IC₅₀ is the concentration at which 50% of the ³H ligand is displaced by the test compound and K_{D} is the dissociation constant for the ³H ligand at the receptor site.

Biological Activity

The Ki values of certain compounds of the present invention in the opioid receptor binding assays were determined, and were found to have Ki values of 4000 nM or less for the μ receptor.

It is believed that the methods used in the following Examples produce compounds having the relative stereochemistry shown below, and such compounds are preferred:

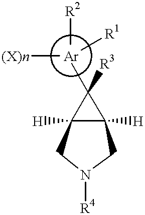

wherein R¹⁻⁴ and (X)ₙ are as defined above.

The invention is illustrated by the following Examples and Preparations in which the following abbreviations may be used:

APCI=atmospheric pressure chemical ionization

DMF=dimethylformamide

DMSO=dimethylsulphoxide

d (in relation to time)=day

d (in relation to NMR)=doublet

ES (in relation to MS)=electrospray

EtOAc=ethyl acetate

EtOH=ethanol

h=hour

MeOH=methanol

min=minute

MS=mass spectrum

n-BuOH=n-butanol

ODS=octadecylsilyl

THF=tetrahydrofuran

TSP=thermospray

Melting points were determined using a Gallenkamp melting point apparatus and are uncorrected. Nuclear magnetic resonance (NMR) spectral data relate to ¹H and were obtained using a Varian Unity 300 or 400 spectrometer, the observed chemical shifts (δ) being consistent with the proposed structures. Mass spectral (MS) data were obtained on a Fisons Instruments Trio 1000, or a Fisons Instruments Trio 1000 APCI, or a Finnigan Navigator MS, or a Micromass Platform LC spectrometer. The calculated and observed ions quoted refer to the isotopic composition of lowest mass. Room temperature means 20 to 25° C. The mass spectrometer which is used as a detector on the analytical HPLC-MS system is a Micromass VG Platform II, running on Masslynx/Openlynx software. The system can run positive and negative ion with either Electrospray or APCI probes and is calibrated to 1972 Daltons, it collects full Diode array data from 190 nm to 600 nm.

HPLC means high performance liquid chromatography. HPLC conditions used were:

Condition 1: Rainin Dynamax™ column, 8μ ODS, 24×300 mm, column temperature 40° C., flow rate 45 ml/min, eluting with methanol:water (70:30), UV detection of product at 246 nm.

Condition 2: Rainin Dynamax™ column, 5μ ODS, 21.6×250 mm, column temperature 40° C., flow rate 5 ml/min, eluting with acetonitrile:water (50:50), UV detection of product at 246 nm.

Condition 3: Rainin Dynamax™ column, 8μ ODS, 41×250 mm, column temperature 40° C., flow rate 45 ml/min, eluting with acetonitrile: 0.1M aqueous ammonium acetate buffer (50:50), UV detection of product at 235 nm.

Condition 4: Phenomenex Magellan™ column, 5μ C₁₈ silica, 21.2×150 mm, column temperature 40° C., flow rate 20 ml/min, eluting with a gradient of acetonitrile: 0.1M aqueous ammonium acetate buffer (30:70 to 95:5 over 10 min), UV detection of product at 220 nm.

Condition 5: Phenomenex MagellanTM column, 5μ ODS, 21.2×150 mm, column temperature 40° C., flow rate 20 ml/min, eluting with a gradient of acetonitrile: 0.1M aqueous ammonium acetate buffer (5:95 to 95:5 over 20 min), UV detection of product at 215 nm.

Condition 6: Phenomenex Magellan™ column, 5μ C₁₈ silica, 4.6×150 mm, column temperature 40° C., flow rate 1 ml/min, eluting with a gradient of acetonitrile: 0.1M aqueous heptanesulphonic acid (10:90 to 90:10 over 30 min), UV detection of product at 220 nm.

Condition 7: Phenomenex Magellan™ column, 5μ C₁₈ silica, 21.2×150 mm, column temperature 40° C., flow rate 20 ml/min, eluting with a gradient of acetonitrile: 0.05M aqueous ammonium acetate buffer (50:50 for 15 min then 50:50 to 90:10 over 5 min), UV detection of product at 220 nm.

Condition 8: Phenomenex Magellen™ column, 5μ C₁₈ silica, 21.2×150 mm, column temperature 40° C., flow rate 20 ml/min, eluting with a gradient of acetonitrile: 0.1M aqueous ammonium acetate buffer (15:85 to 85:15), UV detection of product at 220 nm.

Condition 9: Phenomenex Magellen™ column, 5μ ODS, 10×150 mm, column temperature 40° C., flow rate 5ml/min, eluting with a gradient of acetonitrile: 0.1M aqueous ammonium acetate buffer (5:95 to 30:70 over 5 min then 30: 70 for a further 20 min), UV detection of product at 225 nm.

Condition 10: Phenomenex Magellan™ column, 5μ C₁₈ silica, 21.2×150 mm, column temperature 40° C., flow rate 20 ml/min, eluting with a gradient of acetonitrile: 0.1M aqueous ammonium acetate (5:95 to 40:60 over 5 min then 40:60 for a further 25 min), UV detection of product at 210 nm.

Condition 11: Phenomenex Magellan™ column, 5μ ODS, 10×150 mm, column temperature 40° C., flow rate 5 ml/min, eluting with a gradient of acetonitrile:water (5:95 to 55:45 over 5 min), UV detection of product at 210 nm.

The free base form of the azabicycles could be obtained from the hydrochloride or acetate salts, for example, in the following way. The salt (0.3 mmol) was dissolved in dichloromethane (20 ml) and washed with saturated aqueous sodium hydrogen carbonate solution (20 ml). The basic mixture was separated and the aqueous layer was extracted with dichloromethane (2×20 ml). The combined organic extracts were dried (Na₂SO₄) and concentrated in vacuo to give the free base.

SPE cartridge refers to a solid phase extraction cartridge. These can be commercially obtained from Varian (Mega Bond Elut™) or Isolute NB “Examples” numbered 1-144 are compounds related to the instant invention, but with different R⁴ groups, and are disclosed as such in International Patent Application no. WO00/39089, herein incorporated by reference in its entirety.

A number of further Examples, such as those in the table below, can be made via the processes A-K outlined below and in the experimental details following the table

Process A

Alkylation

Alkylation of the amine of formula VIII or a salt thereof with R⁴Lg, where Lg is a suitable leaving group, such as a halogen, triflate, mesylate, etc., in the presence of a base, optionally in the presence of a catalyst, in a polar solvent, at between 0 and 150° C.

Preferably the alkylation is carried out with R⁴Lg (slight excess), where Lg═Cl or Br, an excess of base (2.0-4.0 eq), such as K₂CO₃, NaHCO₃, or a tertiary amine, such as triethylamine or Hunigs base, in a polar solvent, such as THF, DMF, or MeCN, at between 40 and 120° C., optionally in the presence of a catalyst such as Nal or KI, for 2-24 hr. see R C Larrock in “Comprehensive Organic Transformations-A Guide to Functional Group Preparations”, VCH, (1989), p 397, and references cited therein.

For Example:

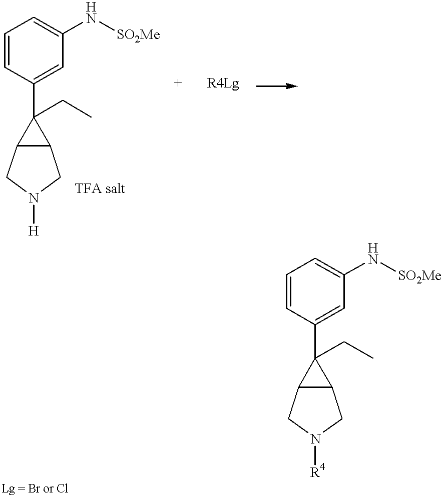

Conditions: Amine salt (1.0 eq), RX (1.1 eq), NaHCO3 (2-4.0 eq), DMF, Nal (cat), 40-120° C.

Process B

Reductive amination

Treating an appropriate aldehyde R^{4a}CHO with an amine of formula VIII in the presence of a suitable reducing agent (such as sodium cyanoborohydride, sodium triacetoxyborohydride, or catalytic hydrogenation with Pd, Pt or Ni catalysts). The reaction is often performed in the presence of acetic acid at 0-100° C. in THF, MeOH, DCM, or DCE (1,2 -dichloroethane), for 1-24 hr.

Preferably the amine salt is treated with an organic base (1-3 eq), such as triethylamine or Hunigs base, and then the aldehyde (1-1.5 eq), followed by sodium triacetoxyborohydride (1-2.Oeq), in dichloromethane or DCE, at room temperature for 2-24 hr. see R C Larrock; “Comprehensive Organic Transformations—A Guide to Functional Group Preparations”, second edition, (1999), p 835-842, and references cited therein, and Abdel-Magid et al, J. Org. Chem., 1996, 61, 3849.

For example:

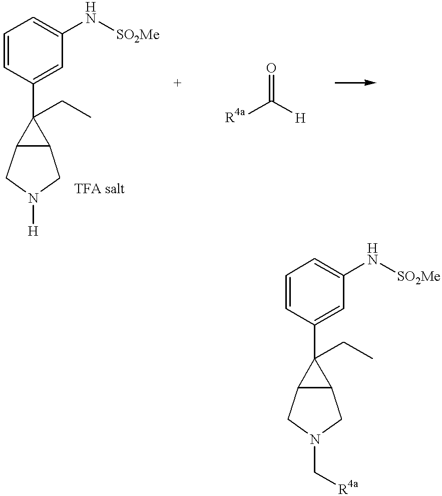

Conditions: Amine salt (1.0 eq), RCHO (1-1.5eq), Et3N (1-3 eq), Na(OAc)3BH (1-2 eq), DCM RT.

Process C

Reduction of Amide of Formula XXXI

The amide carbonyl can be reduced with a suitable reducing agent, for example lithium aluminium hydride or borane, in an ethereal solvent, such as THF, at 0-100° C. to generate the desired tertiary amine, see R C Larrock in “Comprehensive Organic Transformations—A Guide to Functional Group Preparations”, VCH, (1989), pp 432-434, and references cited therein.

Preferably the amide (1.0 eq) is treated with lithium aluminium hydride (1.0-3 eq), at 0° C.-RT, in THF, for 1-24 hr, e.g.:

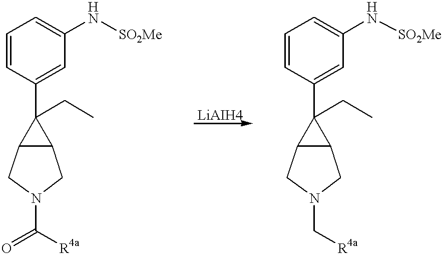

Process D

Oxidation

Aldehydes used in process B can be prepared using suitable oxidising agents; see R C Larrock in “Comprehensive Organic Transformations—A Guide to Functional Group Preparations”, second edition, (1999), pp 1234-1236 and 1238-1247, and references cited therein.

Preferred oxidants are tetrapropylammonium perruthenate (Ley, et.al., Synthesis, 1994, 639-666), Swern oxidation and related methods (Tidwel¹, Organic Reactions, 1990, 39, 297-572), and Dess-Martin Periodinane reagent (Dess et al., J. Org. Chem., 1983, 48, 4155-4156).

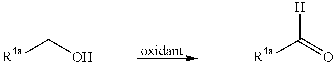

Process E

Acid/amine salt coupling to give amides of formula XXXI

Either using an acid chloride+amine in a suitable solvent or the acid activated by a suitable agent, optionally in the presence of a catalyst, for example DMAP, in a suitable solvent; see R C Larrock in “Comprehensive Organic Transformations—A Guide to Functional Group Preparations”, second edition, (1999), pp 1941-1949, and references cited therein. Preferably the carboxylic acid (0.9-1.1 eq), 1-(3-dimethylaminopropyl)-3-ethylcarbodiimide. HCl (1-1.5 eq) and 1-hydroxybenzotriazole (1.0 eq) are stirred in DMF or DCM at RT for 5-15 min and then the amine salt (1 eq) and base (NaHCO3 or organic base, Et3N or Hunigs base (2-4 eq)) are added, the reaction taking 2-24 hr at RT.

For Example:

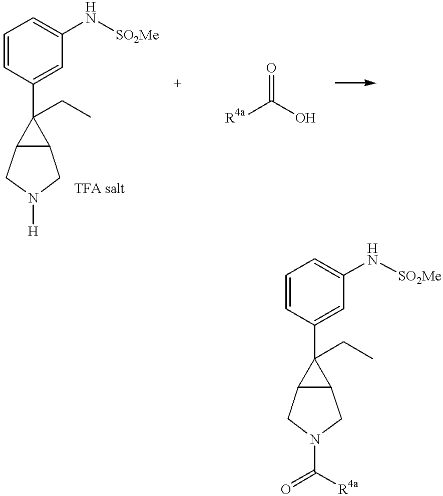

Process F

Urea formation

Anilines can be converted to a urea using potassium cyanate (excess) in an acidic aqueous solution, see Cross et al., J. Med. Chem., 1985, 28, 1427-1432. viz. reaction of the type:

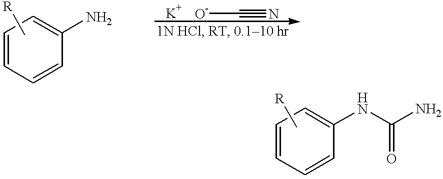

Process G

Ester to an alcohol

Esters can be converted to the corresponding alcohol using a suitable reducing agent, see Larock, Comprehensive Organic Transformations—A Guide to Functional Group Preparations, second edition, (1999), pp 1117-1120 and references cited therein. Suitable reducing agents include diisobutylaluminium hydride (DIBAL, see Winterfeldt, Synthesis, 1975, 617) and lithium aluminium hydride (LiAIH4, see Brown, Org. Reactions, 1951, 6, 469) viz. reaction of the type:

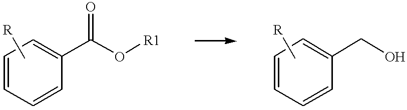

Process H

Acid to alcohol

It should be appreciated that the alcohols used in process D can be prepared from the corresponding acid using a suitable reducing agent; see Larock, Comprehensive Organic Transformations—A Guide to Functional Group Preparations, second edition, (1999), pp 1114-1116. Preferably the reducing agent is either borane (BH3 (1-2 eq), J. Org. Chem., 1973, 38, 2786), or LiAlH4 (1-4 eq), in an ethereal solvent, such as THF, 0-80° C., for 1-24 hr.

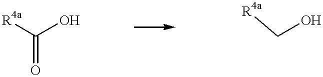

Process I

Alcohol to halide

It should be appreciated that the R⁴Lg used in Process A can be prepared from the corresponding alcohol R^{4a}OH.

Direct methods to prepare alkyl halides and alkyl sulphonates from their alcohols are described by R C Larock, Comprehensive Organic Transformations-A Guide to Functional Group Preparations, second edition, (1999),

pp 689-700, and references cited therein.

Process J

Benzyl halides to benzyloxyalcohols

Benzyloxyalcohols can be prepared by refluxing the appropriate benzyl halide with sodium or sodium hydride and a polymethylene glycol in xylene, see J. Am. Chem. Soc., 1951, 3159-3162.viz. reaction of the type:

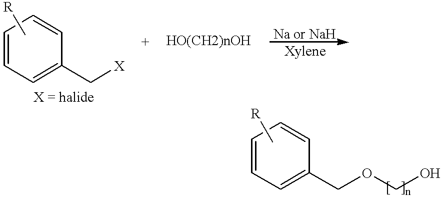

Process K

Acetals to benzyloxyalcohols

Acetals can be treated with a suitable reducing agent in the presence of a Lewis acid or organic acid to give the benyloxyalcohols.

For representative examples see Organic Preparations and Procedures, Int., 1991, 23, 4, 427-431, ZrC_{1-4/}LiAlH4; J. Org. Chem., 1987, 52, 2594, Zn(BH4)2/Me3SiCI; and Organic Preparations and Procedures, Int., 1985, 17(1), 11-16, NaBH4/TFA.viz. reaction of the type:

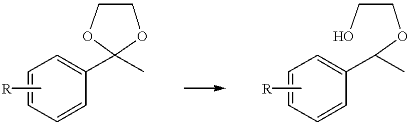

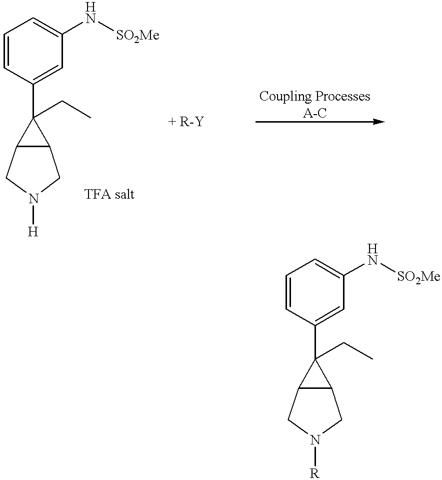

| | | |
|---|---|---|
| | | Precursor(s) |
| Example | Name | Coupling method |

—(CH₂)nO(CH₂)nR examples

| | | |
|---|---|---|
| 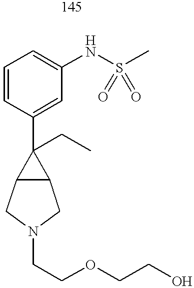 | N-(3-{6-ethyl-3-[2-(2- hydroxyethoxy)ethyl]-3- azabicyclo[3.1.0]hex-6- yl}phenyl)methanesulfona mide | 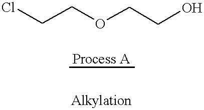 |
| 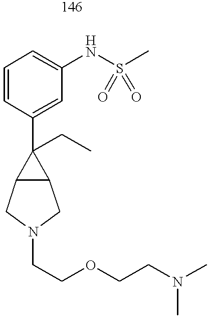 | N-[3-(3-{2-[2- (dimethylamino)ethoxy]et hyl}-6-ethyl-3- azabicyclo[3.1.0]hex-6- yl)phenyl]methanesulfona mide | 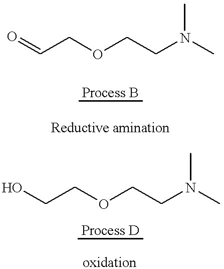 |
| 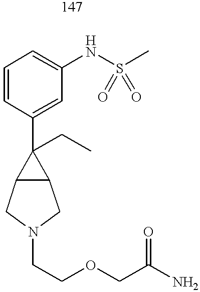 | 2-[2-(6-ethyl-6-{3- [(methylsulfonyl)amino]ph enyl}-3- azabicyclo[3.1.0]hex-3- yl)ethoxy]acetamide | 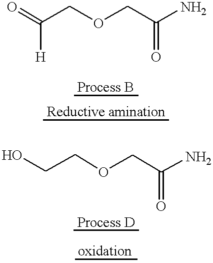 |
| 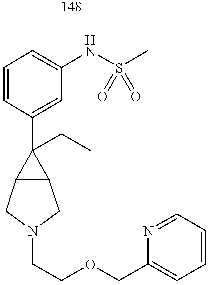 | N-(3-{6-ethyl-3-[2-(2- pyridinylmethoxy)ethyl]-3- azabicyclo[3.1.0]hex-6- yl}phenyl)methanesulfona mide | 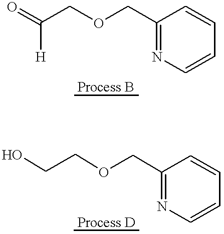 |
| 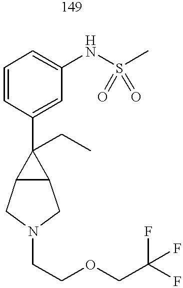 | N-(3-{6-ethyl-3-[2-(2,2,2- trifluoroethoxy)ethyl]-3- azabicyclo[3.1.0]hex-6- yl}phenyl)methanesulfona mide | 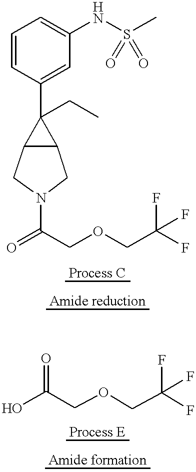 |
| 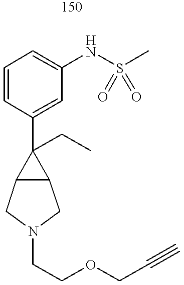 | N-(3-{6-ethyl-3-[2-(2- propynyloxy)ethyl]-3- azabicyclo[3.1.0]hex-6- yl}phenyl)methanesulfona mide | 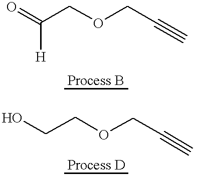 |
| 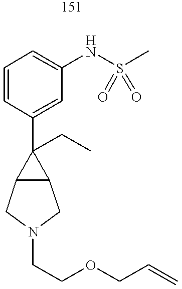 | N-(3-{3-[2-(allyloxy)ethyl]- 6-ethyl-3- azabicyclo[3.1.0]hex-6- yl}phenyl)methanesulfona mide | 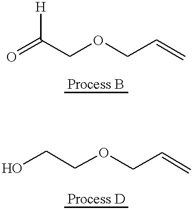 |
| 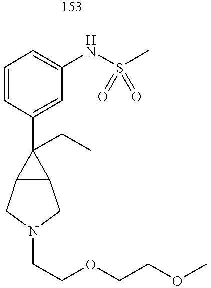 | N-(3-{6-ethyl-3-[2-(2- methoxyethoxy)ethyl]-3- azabicyclo[3.1.0]hex-6- yl}phenyl)methanesulfona mide | 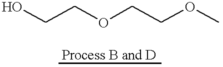 |
| 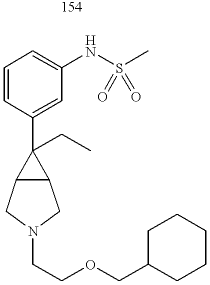 | N-(3-{3-[2- (cyclohexylmethoxy)ethyl]-6-ethyl-3- azabicyclo[3.1.0]hex-6- yl}phenyl)methanesulfona mide | 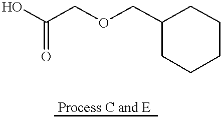 |
| (CH₂)nO(CH₂)nAr examples | | |
| 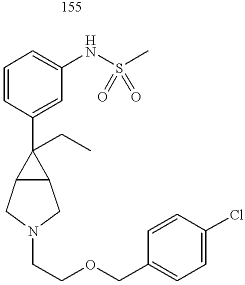 | N-[3-(3-{2-[(4- chlorobenzyl)oxy]ethyl}-6- ethyl-3- azabicyclo[3.1.0]hex-6- yl)phenyl}methanesulfona mide | 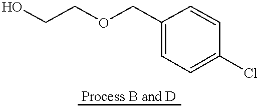 |
| 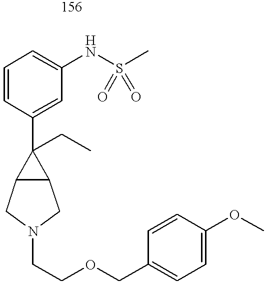 | N-[3-(6-ethyl-3-{2-[(4- methoxybenzyl)oxy]ethyl}-3-azabicyclo[3.1.0]hex-6- yl)phenyl}methanesulfona mide | 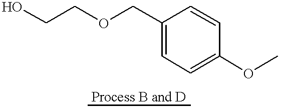 |
| All other (CH₂)nO(CH₂)nAr | | For a general procedure |
| examples can be prepared via a 2 | | see; J. Am. Chem. Soc., |
| step process from a benzyl | | 1951, 3159-3162. |
| alcohol or benzyl chloride | | |
| Aryl substituents (mix. of aryl and | | |
| aryloxy examples): | | |
| 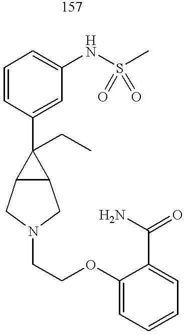 | 2-[2-(6-ethyl-6-{3- [(methylsulfonyl)amino]ph enyl}-3- azabicyclo[3.1.0]hex-3- yl)ethoxy]benzamide | 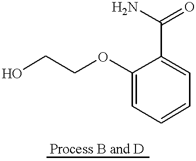 |
| 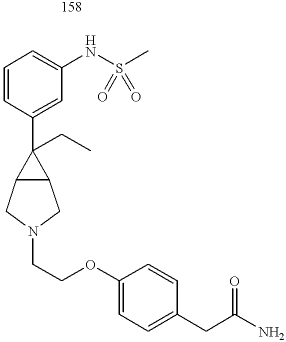 | 2-{4-[2-(6-ethyl-6-{3- [(methylsulfonyl)amino]ph enyl}-3- azabicyclo[3.1.0]hex-3- yl)ethoxy]phenyl}acetami de | 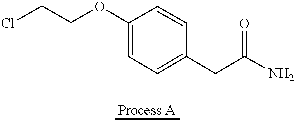 |
| 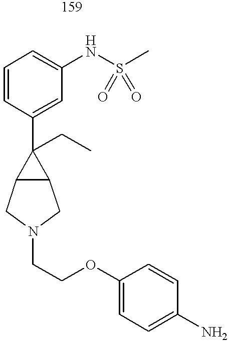 | N-(3-{3-[2-(4- aminophenoxy)ethyl]-6- ethyl-3- azabicyclo[3.1.0]hex-6- yl}phenyl)methanesulfona mide | 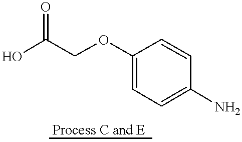 |
| 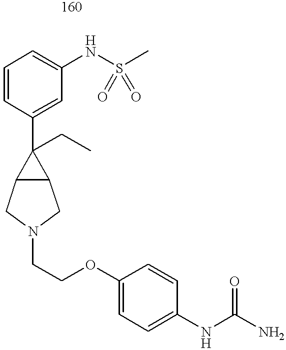 | N-{3-[3-(2-{4- [(aminocarbonyl)amino]p henoxy}ethyl)-6-ethyl-3- azabicyclo[3.1.0]hex-6- yl]phenyl}methanesulfona mide | 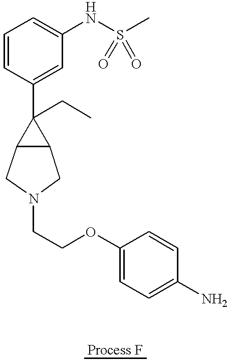 |
| 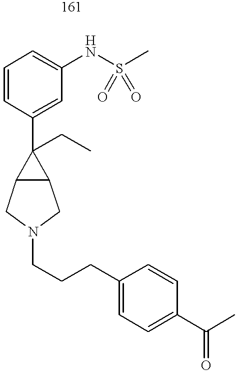 | N-(3-{3-[3-(4- acetylphenyl)propyl]-6- ethyl-3- azabicyclo[3.1.0]hex-6- yl}phenyl)methanesulfona mide | 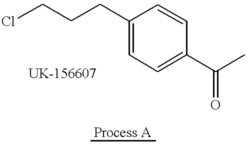 |
| 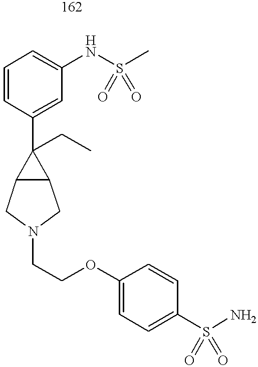 | 4-[2-(6-ethyl-6-{3- [(methylsulfonyl)amino]ph enyl}-3- azabicyclo[3.1.0]hex-3- yl)ethoxy]benzenesulfona mide | 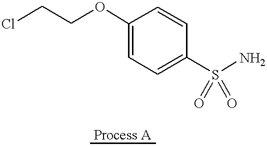 |
| 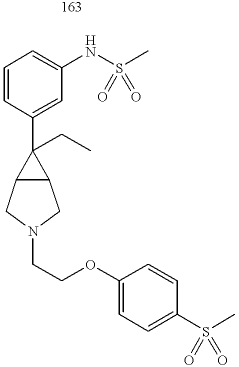 | N-[3-(6-ethyl-3-{2-[4- (methylsulfonyl)phenoxy]ethyl}-3- azabicyclo[3.1.0]hex-6- yl)phenyl]methanesulfona mide | 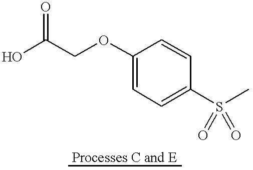 |
| 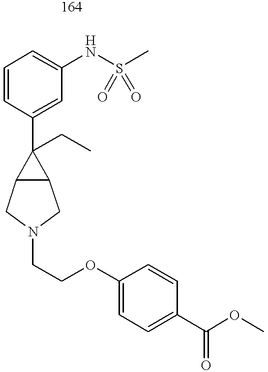 | methyl 4-[2-(6-ethyl-6-{3- [(methylsulfonyl)amino]ph enyl}-3- azabicyclo[3.1.0]hex-3- yl)ethoxy]benzoate | 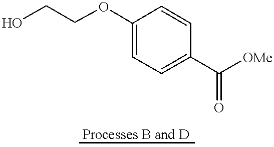 |
| 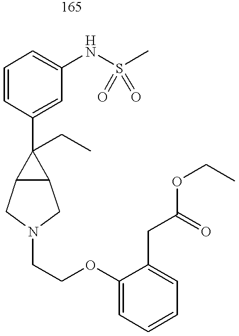 | ethyl {2-[2-(6-ethyl-6-{3- [(methylsulfonyl)amino]ph enyl}-3- azabicyclo[3.1.0]hex-3- yl)ethoxy]phenyl}acetate | 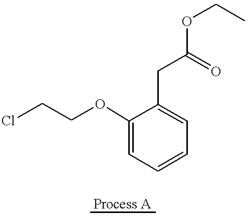 |
| 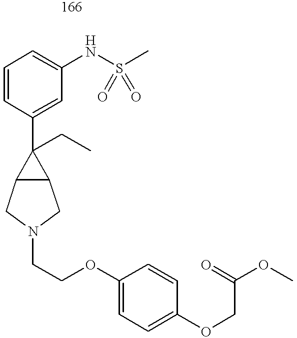 | methyl {4-[2-(6-ethyl-6-{3- [(methylsulfonyl)amino]ph enyl}-3- azabicyclo[3.1.0]hex-3- yl)ethoxy]phenoxy}acetate | 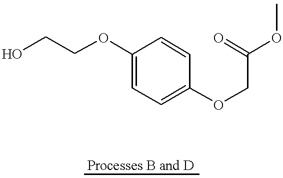 |
| 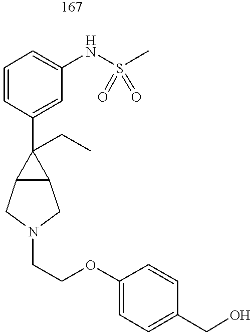 | N-[3-(6-ethyl-3-{2-[4- (hydroxymethyl)phenoxy]ethyl}-3- azabicyclo[3.1.0]hex-6- yl)phenyl]methanesulfona mide | 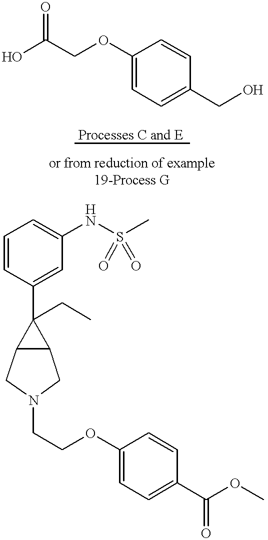 |
| 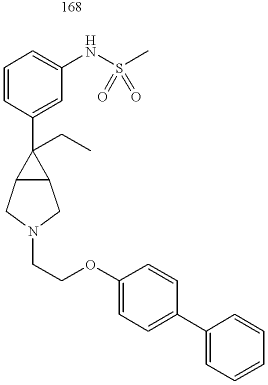 | N-(3-{3-[2-([1,1′- biphenyl]-4-yloxy)ethyl]-6- ethyl-3- azabicyclo[3.1.0]hex-6- yl)phenyl)methanesulfona mide | 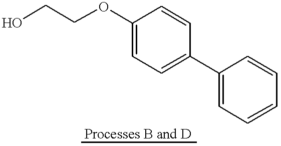 |
| 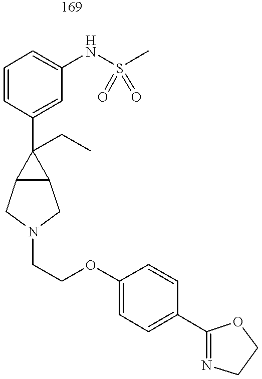 | N-[3-(3-{2-[4-(4,5-dihydro- 1,3-oxazol-2- yl)phenoxy]ethyl}-6-ethyl- 3-azabicyclo[3.1.0]hex-6- yl)phenyl]methanesulfona mide | 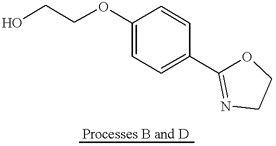 |
| 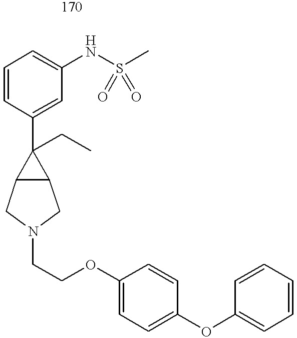 | N-(3-{6-ethyl-3-[2-(4- phenoxyphenoxy)ethyl]-3- azabicyclo[3.1.0]hex-6- yl}phenyl)methanesulfona mide | 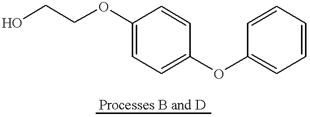 |
| 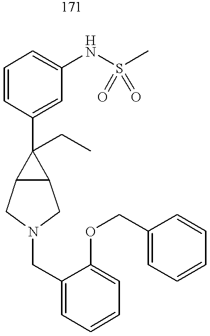 | N-(3-{3-[2- (benzyloxy)benzyl]-6- ethyl-3- azabicyclo[3.1.0]hex-6- yl}phenyl)methanesulfona mide | Commercial aldehyde Processes B |
| 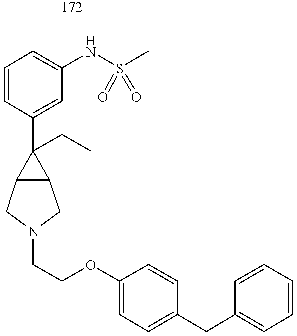 | N-(3-{3-[2-(4- benzylphenoxy)ethyl]-6- ethyl-3- azabicyclo[3.1.0]hex-6- yl}phenyl)methanesulfona mide | 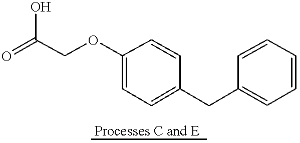 |
| 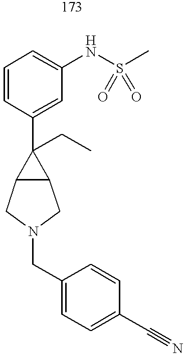 | N-{3-[3-(4-cyanobenzyl)- 6-ethyl-3- azabicyclo[3.1.0]hex-6- yl]phenyl}methanesulfona mide | Commercial aldehyde Process B |
| 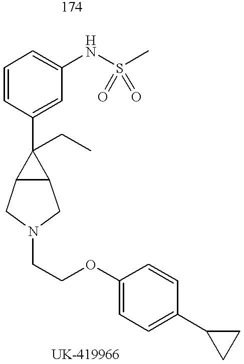 | N-(3-{3-[2-(4- cyclopropylphenoxy)ethyl]-6-ethyl-3- azabicyclo[3.1.0]hex-6- yl}phenyl)methanesulfona mide | 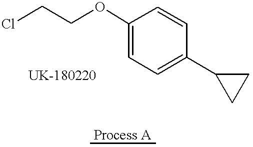 |
| Substituents of the basic | | |
| alkyl/alkenyl/alkynyl chains: | | |
| 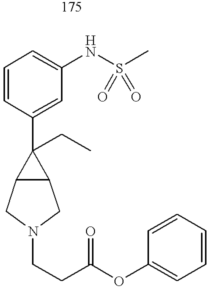 | phenyl 3-(6-ethyl-6-{3- [(methylsulfonyl)amino]ph enyl}-3- azabicyclo[3.1.0]hex-3- yl)propanoate | 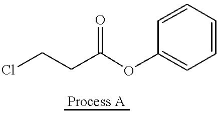 |
| 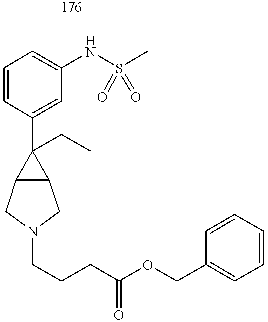 | benzyl 4-(6-ethyl-6-{3- [(methylsulfonyl)amino]ph enyl}-3- azabicyclo[3.1.0]hex-3- yl)butanoate | 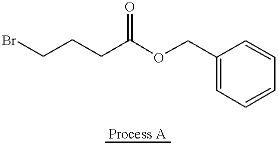 |
| 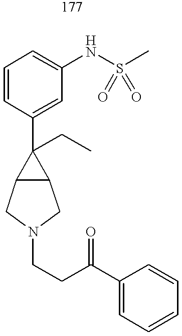 | N-{3-[6-ethyl-3-(3-oxo-3- phenylpropyl)-3- azabicyclo[3.1.0]hex-6- yl]phenyl}methanesulfona mide | 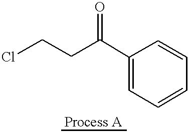 |
| 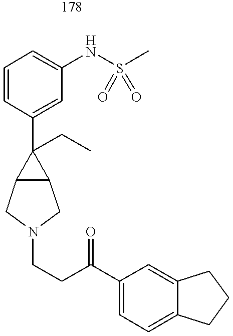 | N-(3-{3-[3-(2,3-dihydro- 1H-inden-5-yl)-3- oxopropyl]-6-ethyl-3- azabicyclo[3.1.0]hex-6- yl}phenyl)methanesulfona mide | 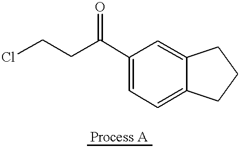 |
| 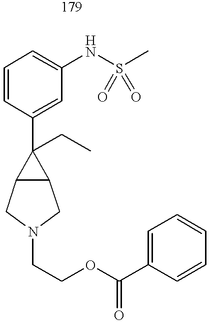 | 2-(6-ethyl-6-{3- [(methylsulfonyl)amino]ph enyl}-3- azabicyclo[3.1.0]hex-3- yl)ethyl benzoate | 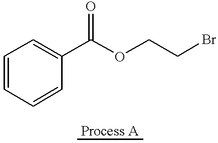 |
| 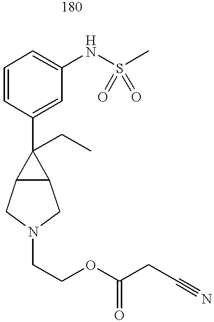 | 2-(6-ethyl-6-{3- [(methylsulfonyl)amino]ph enyl}-3- azabicyclo[3.1.0]hex-3- yl)ethyl cyanoacetate | 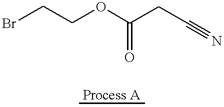 |
| 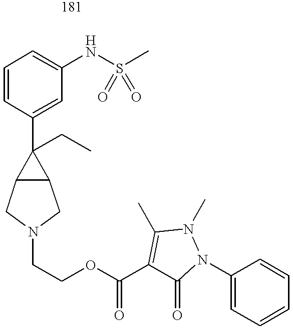 | 2-(6-ethyl-6-{3- [(methylsulfonyl)amino]ph enyl}-3- azabicyclo[3.1.0]hex-3- yl)ethyl 1,5-dimethyl-3- oxo-2-phenyl-2,3-dihydro- 1H-pyrazole-4- carboxylate | 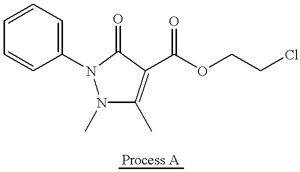 |
| 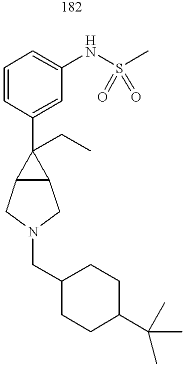 | N-(3-{3-[(4-tert- butylcyclohexyl)methyl]-6- ethyl-3- azabicyclo[3.1.0]hex-6- yl}phenyl)methanesulfona mide | 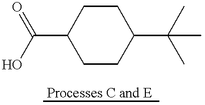 |
| 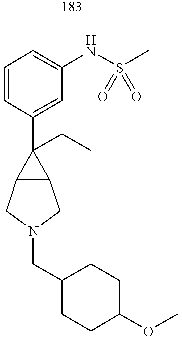 | N-(3-{6-ethyl-3-[(4- methoxycyclohexyl)methy l]-3-azabicyclo[3.1.0]hex- 6- yl}phenyl)methanesulfona mide | 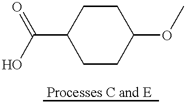 |
| 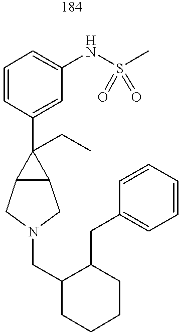 | N-(3-{3-[(2- benzylcyclohexyl)methyl]6-ethyl-3- azabicyclo[3.1.0]hex-6- yl}phenyl)methanesulfona mide | 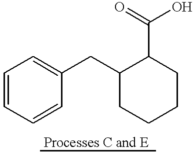 |
| 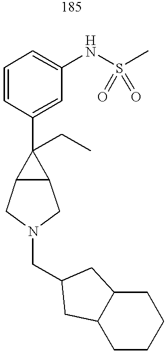 | N-{3-[6-ethyl-3- (octahydro-1H-inden-2- ylmethyl)-3- azabicyclo[3.1.0]hex-6- yl]phenyl)methanesulfona mide | 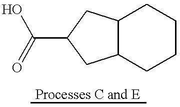 |
| 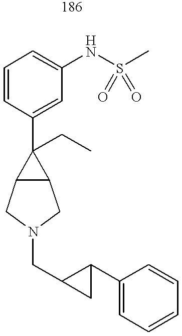 | N-(3-{6-ethyl-3-[(2- phenylcyclopropyl)methyl]-3-azabicyclo[3.1.0]hex-6- yl}phenyl)methanesulfona mide | 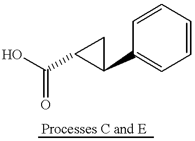 |
| 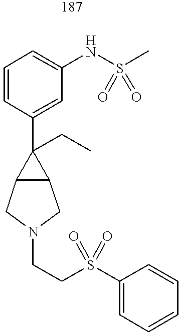 | N-(3-{6-ethyl-3-[2- (phenylsulfonyl)ethyl]-3- azabicyclo[3.1.0]hex-6- yl}phenyl)methanesulfona mide | 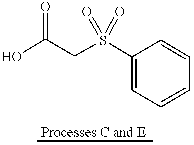 |
| 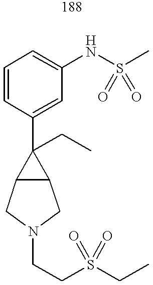 | N-(3-{6-ethyl-3-[2- (ethylsulfonyl)ethyl]-3- azabicyclo[3.1.0]hex-6- yl}phenyl)methanesulfona mide | 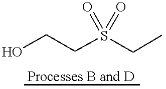 |
| 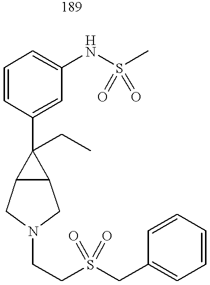 | N-(3-{3-[2- (benzylsulfonyl)ethyl]-6- ethyl-3- azabicyclo[3.1.0]hex-6- yl}phenyl)methanesulfona mide | 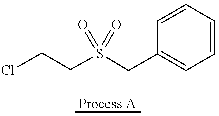 |
| 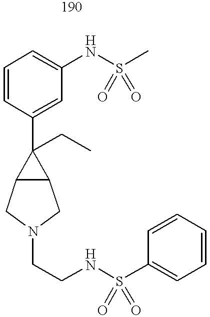 | N-[2-(6-ethyl-6-{3- [(methylsulfonyl)amino]ph enyl}-3- azabicyclo[3.1.0]hex-3- yl)ethyl]benzenesulfonam ide | 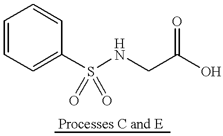 |
| 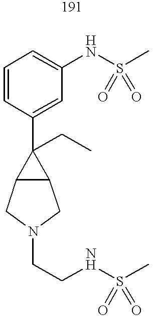 | N-[3-(6-ethyl-3-{2- [(methylsulfonyl)amino]et hyl}-3- azabicyclo[3.1.0]hex-6- yl)phenyl}methanesulfona mide | 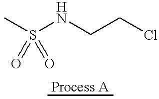 |
| 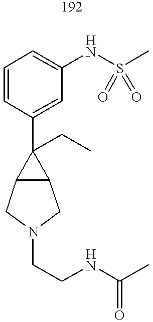 | N-[2-(6-ethyl-6-{3- [(methylsulfonyl)amino]ph enyl}-3- azabicyclo[3.1.0]hex-3- yl)ethyl]acetamide | 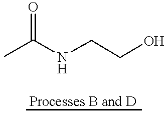 |
| 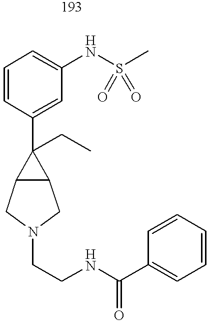 | N-[2-(6-ethyl-6-{3- [(methylsulfonyl)amino]ph enyl}-3- azabicyclo[3.1.0]hex-3- yl)ethyl]benzamide | 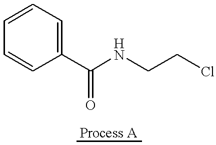 |
| 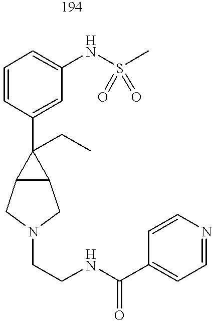 | N-[2-(6-ethyl-6-{3- [(methylsulfonyl)amino]ph enyl}-3- azabicyclo[3.1.0]hex-3- yl)ethyl]isonicotinamide | 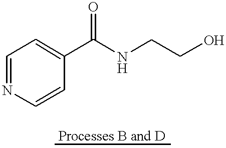 |
| 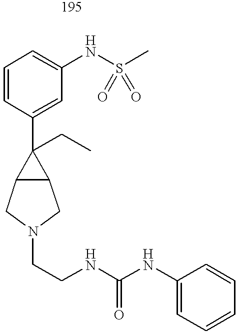 | N-[3-(3-{2- [(anilinocarbonyl)amino]et hyl}-6-ethyl-3- azabicyclo[3.1.0]hex-6- yl)phenyl}methanesulfona mide | 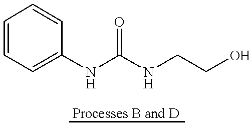 |
| 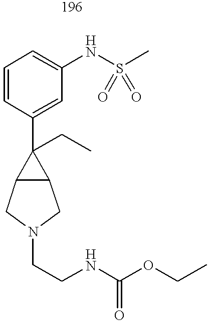 | ethyl 2-(6-ethyl-6-{3- [(methylsulfonyl)amino]ph enyl}-3- azabicyclo[3.1.0]hex-3- yl)ethylcarbamate | 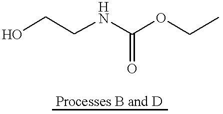 |
| 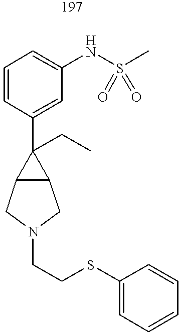 | N-(3-{6-ethyl-3-[2- (phenylsulfanyl)ethyl]-3- azabicyclo[3.1.0]hex-6- yl}phenyl)methanesulfona mide | 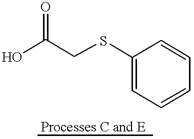 |
| 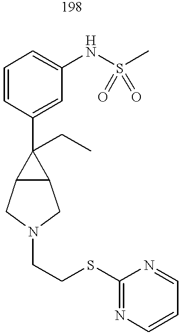 | N-(3-{6-ethyl-3-[2-(2- pyrimidinylsulfanyl)ethyl]- 3-azabicyclo[3.1.0]hex-6- yl}phenyl)methanesulfona mide | 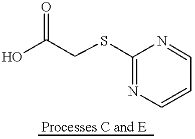 |

### EXAMPLE 199

### :N-(3-{3-[3-(4-acetylphenyl)propyl]-6-ethyl-3-azabicyclo[3.1.0)hex-6-ylphenyl)methanesulfonamide- and formate salt

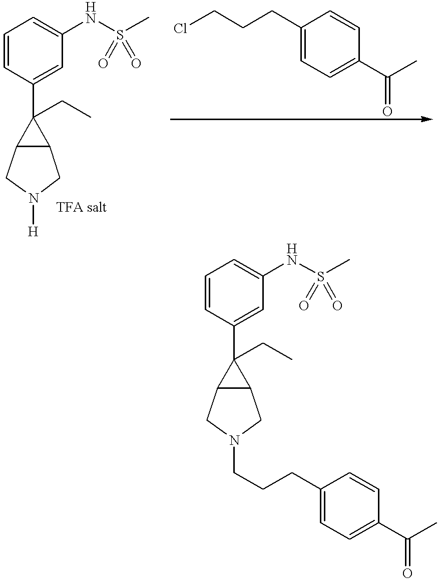

To a solution of the trifluoroacetic acid salt of N-[3-(6-ethyl-3-azabicyclo[3.1.0]hex-6-yl)phenyl]methanesulfonamide (106 mg, 0.27 mmol) in N,N- dimethylformamide (4 ml) was added sodium hydrogen carbonate (9Omg, 1.1 mmol), 1-[4-(3-chloropropyl)phenyl]ethanone (58 mg, 0.29 mmol) and sodium iodide (catalytic) and the reaction mixture was heated at 70° C. for 20 h. After cooling, the solvent was removed in-vacuo to give a crude residue. This was purified by silica (14 g) column chromatography eluting with ethyl acetate:hexane (75:25) and then with neat ethyl acetate. Combination and evaporation of the appropriate fractions gave the partially purified product. This material was further purified by preparative HPLC (condition 1) to afford the formate salt of title compound (16 mg, 12%) as a yellow oil.

¹H-NMR (300MHz, CDCl₃, data for formate salt) : 0.85 (t, 3H), 1.70 (q, 2H), 2.05 (quintet, 2H), 2.15 (s, 2H), 2.55 (s, 3H), 2.70 (t, 2H), 2.80-2.85 (m, 4H), 2.95 (s, 3H), 3.70-3.80 (m, 2H), 7.00 (d, 1H), 7.05-7.10 (m, 2H), 7.20-7.28 (m, 3H), 7.90 (d, 2H), 8.40 (s, 1H).

MS (Electrospray): M/Z (M-H) 439; C₂₅H₃₂N₂O₃S—H requires 439.2.

### EXAMPLE 200

### :N-(3-{3-[2-(benzyloxy)benzyl]-6-ethyl-3-azabicyclo[3.1.0]hex-6-yl}phenyl)methanesulfonamide

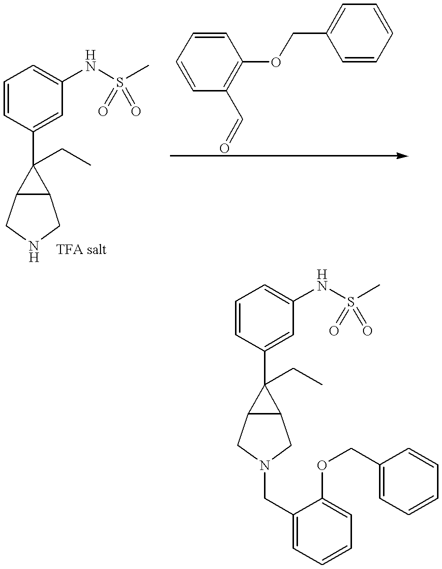

To a solution of 2-benzyloxybenzaldehyde (27mg, 0.13 mmol) in dichloromethane (5 ml) at room temperature was added the trifluoroacetic acid salt N-[3-(6-ethyl-3-azabicyclo[3.1.0]hex-6-yl)phenyl]methanesulfonamide (50mg, 0.13 mmol) and triethylamine (0.05 ml, 0.38 mmol). The reaction was left to stir at room temperature for 2 h. At this point sodium triacetoxyborohydride (40.8 mg, 0.19 mmol) was added and the reaction was left to stir at room temperature for 16 h. Water (5 ml) was then added to the reaction mixture and the two layers were separated using a Whatman filter tube (hydrophobic polytetrafluoroethylene membrane). The organic layer was then blown down to dryness under a steam of nitrogen. The residue was purified by column chromatography using a Sep-PakTM cartridge packed with silica gel (10 g) eluting with hexane: ethyl acetate (100:0, 1:1, 1:3, 1:6, 1:9 and 0:100) to afford the title compound (28 mg, 46%) as an oil.

¹H-NMR (300 MHz, CDCl₃): 0.85 (t, 3H), 2.80 (s, 2H), 2.00-2.10 (m, 2H), 2.85 (d, 2H), 3.00 (s, 3H), 3.10-3.20 (dd, 2H), 3.80 (s, 2H), 5.10 (s, 2H), 6.90-7.05 (m, 3H), 7.10 (m, 2H), 7.20-7.30 (m, 3H), 7.40-7.50 (m, 6H).

MS (Electrospray): M/Z (M+H) 477; C₂₈H₃₂N₂O₃S+H requires 477.

### EXAMPLE 201

### N-{3-[3-(4-cyanobenzyl)-6-ethyl-3-azabicyclo[3.1.0]hex-6-yl]phenyl}methanesulfonamide

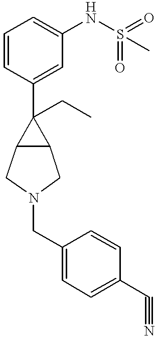

The compound above was prepared by a similar method to that of Example 167, using the trifluoroacetic acid salt of N-[3-(6-ethyl-3-azabicyclo[3.1.0]hex-6-yl)phenyl]methanesulfonamide (100 mg, 0.25 mmol) and 4-cyanobenzaldehyde (33 mg, 0.25 mmol) as the starting materials. The product was purified using preparative HPLC (conditions 3) to afford the title compound (28 mg, 28%) as an off-white solid.

¹H-NMR (300 MHz, CDCl₃): 0.85(t, 3H), 1.80 (s, 2H), 2.05 (q, 2H), 2.80 (d, 2H), 3.00 (s, 3H), 3.10 (d, 2H), 3.70 (s, 2H), 7.00-7.20 (m, 3H), 7.20 (m, 1H), 7.40 (d, 2H), 7.60 (d, 2H)

MS (Electrospray) : M/Z (M+H) 396; C₂₂H₂5N₃O₂S—H requires 396.

### EXAMPLE 202

### N-(3-{3-[2-(4-cyclopropylphenoxy)ethyl]-6-ethyl-3-azabicyclo[3.1.0]hex-6-ylphenyl)methanesulfonamide

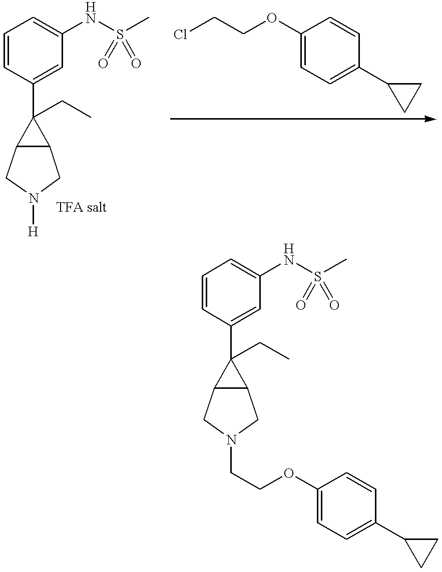

To a solution of the trifluoroacetic acid salt of N-[3-(6-ethyl-3-azabicyclo[3.1.0]hex-6-yl)phenyl]methanesulfonamide (75 mg, 0.19 mmol) in N,N- dimethylformamide (3 ml) was added sodium hydrogen carbonate (64 mg, 0.8 mmol), 1-(2-chloroethoxy)-4-cyclopropylbenzene (41 mg, 0.21 mmol) and sodium iodide (3 mg, catalytic) and the reaction mixture was heated at 60 ° C. for 20 h. After cooling, the solvent was removed in-vacuo to give a crude residue. This was purified by preparative HPLC (condition 2) to afford the formate salt of the title compound (4 mg, 5%) as a brown gum.

¹H-NMR (300 MHz, CDCl₃, data for formate salt): 0.55-0.60 (m, 2H), 0.80-0.95 (m, 5H), 1.80-1.90 (m, 3H), 2.25 (bs, 2H), 2.95 (s, 3H), 3.15 (d, 2H), 3.45 (t, 2H), 3.80-3.90 (m, 2H), 4.20 (t, 2H), 6.90 (d, 2H), 7.00 (d, 2H), 7.05-7.15 (t, 2H), 7.20 (s, 1H), 7.30 (t, 1H).

MS (Electrospray) : M/Z (M-H) 439; C₂₅H₃₂N₂O₃S—H requires 439.2.

### EXAMPLE 203

### N-(3-{6-ethyl-3-[(2-phenylcyclopropyl)methyl]-3-azabicyclo[3.1.0]hex-6-yl}phenyl)methanesulfonamide

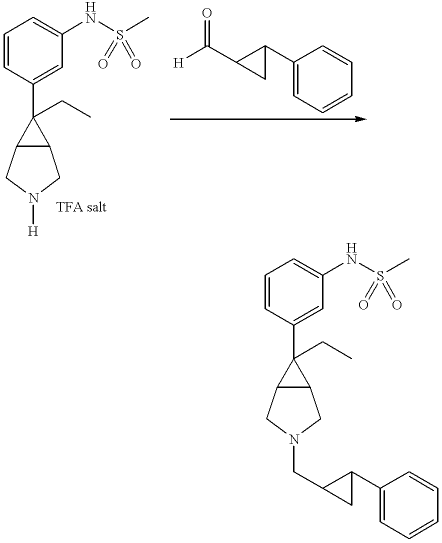

To a mixture of trans-2-phenylcyclopropylcarboxaldehyde {ref. J. Org. Chem., 1992, 57, 1526} (30 mg, 0.2 mmol) and the trifluoroacetic acid salt of N-[3-(6-ethyl-3-azabicyclo[3.1.0]hex-6-yl)phenyl]methanesulfonamide (50 mg, 0.13 mmol) in dry 1,2-dichloroethane was added Hunigs' base (0.02 ml, 0.12 mmol). The mixture was sonicated for 3 minutes and then stirred for a further 30 minutes followed by the addition of sodium triacetoxyborohydride (50 mg, 0.25 mmol). After stirring for 72 hours, the reaction was diluted with ethyl acetate (50ml) and partitioned between saturated sodium bicarbonate (2×25 ml). The organic layer was washed with brine (2×20ml), dried over anhydrous sodium sulphate, filtered and the solvent evaporated under reduced pressure to produce a yellow/brown oil. This oil was dissolved in the minimum of quantity of dichloromethane and purified using a Biotage™ 6 g cartridge eluting with a gradient of ethyl acetate:hexane (30:70) to ethyl acetate (100%) to afford the title compound (32 mg 62%) as an oil.

¹H-NMR (300 MHz, CDCl₃): 0.78-0.90 (m, 3H), 0.97 (m, 1H), 1.24 (m, 1H), 1.72 (m, 1H), 1.76-1.79 (m, 2H) 1.90-2.05 (m, 2H) 2.45 (dd, I H), 2.60 (dd, 1 H), 2.84-2.95 (m, 2H), 2.99 (s, 3H), 3.02-3.08 (m, 2H) 6.89-7.3 (m, 9H).

MS (Electrospray): M/Z (M+H) 41 1; C₂₋₄H₃₀SO₂N₂+H requires 411

### PREPARATIONS

NB Preparations 1 to 148 from International Patent Application publication no. WO00/39089 are herein incorporated by reference in their entirety, and the same numbering is adhered to herein.

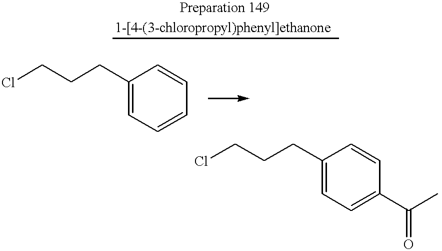

Aluminium chloride (15.0 g, 0.11 moles) and acetyl chloride (16.0 g, 0.20 moles) were dissolved in dichloromethane (50 ml) at room temperature. This mixture was then added dropwise to a solution 1-chloro-3-phenylpropane (15.5 g, 0.10 moles) in dichloromethane (25 ml) at room temperature over 15 minutes. The mixture was stirred for 1 hr and then poured cautiously onto ice. The aqueous layer was extracted with dichloromethane (450 ml). The organics were washed with water and brine, and then dried (MgSO₄) and concentrated in-vacuo to give the title compound (19.2 g, 98%) as an oil.

¹H-NMR (300 MHz, CDCl₃): 2.10 (quintet, 2H), 2.60 (s, 3H), 2.85 (t, 2H), 3.55 (t, 2H), 7.30 (d, 2H), 7.90 (d, 2H).

MS (thermospray): M/Z [M+NH4]⁺214; C₁₁H₁₃ClO+NH₄ requires 214.1.

### PREPARATION 150

### 1-(2-chloroethoxy)-4-cyclopropylbenzene

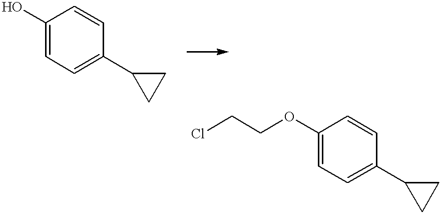

4-Cyclopropylphenol (6.75 g, 50.3 mmol, reference: Horrom et. al., Org. Prep. Proceed. Int., 1992, 24 (6), 696-698), 2-chloroethyl p-toluenesulfonate (17.71 g, 75.5 mmol), and potassium carbonate (10.4 g, 75.4 mmol) in anhydrous acetonitrile (500 ml) were stirred together under a nitrogen atmosphere at reflux for 30 hours. The reaction was allowed to cool to room temperature and diluted with ethyl acetate (1000 ml). The organics were washed with water (3×250 ml), dried (MgSO4), filtered and concentrated in vacuo. This crude material was purified by silica column chromatography eluting with hexane:dichloromethane (4:1) and then with hexane : dichloromethane (3:1) to afford the title compound (8.7 g, 88%) as a solid.

Mpt: 47-48° C.

¹H-NMR (300 MHz, CDCl₃): 0.60-0.70 (m, 2H), 0.85-0.95 (m, 2H), 1.80-1.95 (m, 1H), 3.81 (t, 2H), 4.21 (t, 2H), 6.82 (d, 2H), 7.02 (d, 2H). MS (thermospray) M/Z (M) 196; C₁₁H₁₃OCl requires 196.1.

### PREPARATION 151

1-Allyl-1H-pyrolle-2,5-dione (see J. Org. Chem., 1997, 62, 2652)

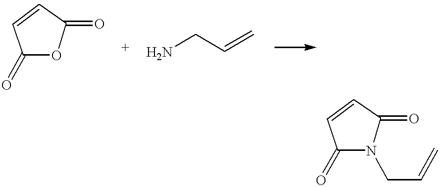

To a solution of maleic anhydride (98 g, 1.00 mol) in dry toluene (3000 ml) at room temperature under a nitrogen atmosphere was added dropwise a solution of allylamine (57.1 g, 1.00 mol) in toluene (1000 ml) over one hour. The mixture was stirred at room temperature for 20 hours and then zinc chloride (136.3 g, 1.00 mol) was added and the reaction was heated to 80° C. 1,1,1,3,3,3-Hexamethyidisilazane (242 g, 1.5 mol) in toluene (1000 ml) was then added dropwise over one hour and the mixture was stirred at 80° C. for another 4 hours. The mixture was cooled to room temperature and then poured onto 1 N HCL (4000 ml). The two layers were separated and the organic layer was washed with water (2000 ml), saturated sodium bicarbonate (2000 ml) and brine (2000 ml). The organics were concentrated in vacuo to give the title compound (74 g, 54%) as a solid.

¹H-NMR (300 MHz, CDCl₃): 4.05 (d, 2H), 5.00-5.15 (m, 2H), 5.60-5.80 (m, 1H), 6.65 (2H, s).

### PREPARATION 152

1-(3-nitrophenyl)-1-propanone hydrazone

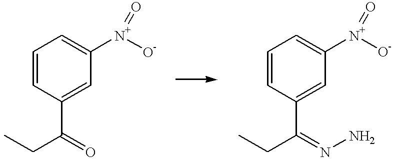

To a solution of 3-nitropropiophenone (168 g, 0.93 mol) in ethanol (830 ml) at room temperature was slowly added hydrazine monohydrate (96.8 g, 1.93 mol) via a dropping funnel. The reaction mixture was heated at reflux for 4 hours and then cooled to room temperature. The solvent was removed in vacuo and the residue was partitioned between dichloromethane (750 ml) and water (750 ml). The two layers were separated and the organic layer was washed with brine (250 ml), dried (Na2SO4), filtered and concentrated in-vacuo to give an orange oil. This residue was crystallised from diisopropyl ether at −20° C. to afford the title compound (110 g, 61%) as a yellow crystalline solid.

Mpt: 32° C.

¹H-NMR (300 MHz, CDCl₃): 1.20 (t, 3H), 2.70 (q, 2H), 5.65 (broad s, 2H), 7.50 (t, 1H), 7.95 (d, 1H), 8.10 (d, 1H), 8.50 (s, 1H).

MS (Electrospray) M/Z [MH]⁺194; C₉H₁₁N₃O₂+H requires 194.1.

### PREPARATION 153

3-Allyl-6-ethyl-6-(3-nitrophenyl)-3-azabicyclo[3.1.0]hexane-2,4-dione

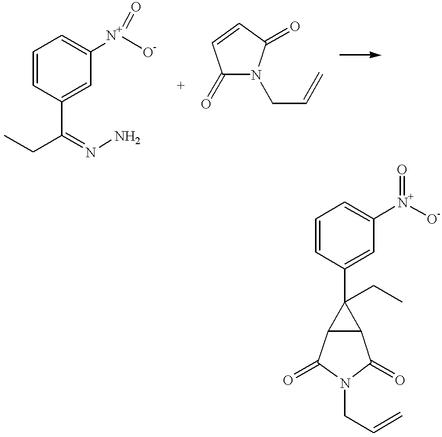

To a stirred solution of 1-(3-nitrophenyl)-1-propanone hydrazone (84.7 9, 439 mmol) in 1,4-dioxane (1000 ml) was rapidly added manganese dioxide (grade CMD-1 from Sumitromo, 175 g, 2.01 mol) followed by a saturated solution of ethanolic potassium hydroxide (40 ml) at room temperature. The mixture was stirred at room temperature for 18 minutes and during this period the reaction temperature had risen from 19° C. to 25° C. Stirring was then stopped and the mixture was allowed to settle. This mixture was then filtered through a pad of Celite® dropwise, directly into a solution of 1 -allyl-1 H-pyrolle-2,5-dione (57.3 g, 418 mmol) in 1,4-dioxane (200 ml). The Celite® pad was washed with 1,4-dixane (100 ml) to ensure complete addition of the reactants. After stirring at room temperature for one hour the mixture was heated at reflux for 20 hours. The mixture was cooled to room temperature and the solvent iwas removed in vacuo. The residue was then crystallised from diisopropyl ether (1000 ml) at 0C to afford the title compound (83 g, 66%) as an off-white crystalline solid.

MPt: 128-129° C.

¹H-NMR (300 MHz, CDCl₃): 0.90 (t, 3H), 1.80 (q, 2H), 2.80 (s, 2H), 4.05 (d, 2H), 5.20 (d, 1H), 5.30 (d, 1H), 5.75-5.85 (m, 1H), 7.55 (t, 1H), 7.70 (dd, 1H), 8.20 (dd, 1H), 8.25 (s, 1H).

### PREPARATION 154

3-allyl-6-(3-aminophenyl)-6-ethyl-3-azabicyclo[3.1.0]hexane-2,4-dione

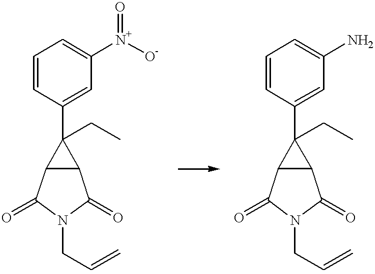

To a stirred suspension of 3-allyl-6-ethyl-6-(3-nitrophenyl)-3-azabicyclo[3.1.0]hexane-2,4-dione (93 g, 310 mmol) and iron powder (151 g, 2.70 mol) in ethanol (6.75 L) was added calcium chloride (16.7 g, 0.15 mol) in water (1.2 L). The mixture was heated at reflux for three hours and then cooled to room temperature before being filtered through Celite®. The filtrate was concentrated in vacuo to give a wet solid. This material was dissolved in dichloromethane (500 ml) and the two layers were separated. The organic layer was dried (MgSO4), filtered and concentrated in vacuo to give a pale yellow solid (81 g). This material was crystallised from ethyl acetate and hexane (1:1; 6 ml per gram) at room temperature to afford the title compound (54 g, 65%) as a pale yellow crystalline solid.

¹H-NMR (300 MHz, CDCl₃): 0.90 (t, 3H), 1.75 (q, 2H), 2.75 (s, 2H), 3.95 (broad s, 2H), 4.05 (d, 2H), 5.25 (d, 1H), 5.35 (d, 1H), 5.75-5.85 (m, 1H), 6.65 (d, 1H), 6.70 (s, 1 H), 6.75 (d, 1H), 7.10 (t, 1H).

### PREPARATION 155

3-(3-Allyl-6-ethyl-3-azabicyclo[3.1.0]hex-6-yl )aniline

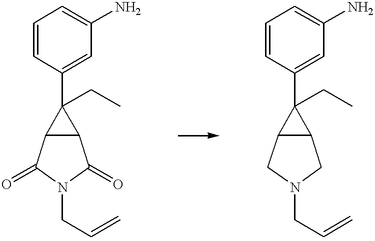

To a solution of lithium aluminium hydride (1 M solution in THF; 400 ml, 400 mmol) in tetrahydrofuran (400 ml) under a nitrogen atmosphere at -1 5° C. was added 3-allyl-6-(3-aminophenyl)-6-ethyl-3-azabicyclo[3.1.0]hexane-2,4-dione (44 g, 163 mmol) in tetrahydrofuran (250 ml) via a dropping funnel over 0.5 hours. The mixture was then allowed to slowly warm to room temperature over one hour. The mixture was heated at 50° C. for 3 hours and then cooled to 5° C. Water (400 ml) was then cautiously added to the cooled (5° C.) reaction mixture. The solids were removed by filtration through a pad of Celite®, washing with ethyl acetate (400 ml). The filtrate was dried (MgSO₄), filtered, and concentrated in vacuo to afford the title compound (38.1 g, 96%) as a golden oil.

¹H-NMR (300 MHz, CDCl₃): 0.85 (t, 3H), 1.80-1.95 (m, 4H), 2.85-3.00 (m, 4H), 3.15 (d, 2H), 3.60 (broad s, 2H), 5.10 (d, 1H), 5.20 (d, 1H), 5.80-5.95 (m, 1H), 6.50 (d, 1H), 6.60 (s, 1H), 6.65 (d, 1H), 7.05 (t, 1H).

MS (AP⁺) M/Z [MH]+243; C₁₆H₂₂N₂+H requires 243.2.

### PREPARATION 156

N-[3-(3-allyl-6-ethyl-3-azabicyclo[3.1.0]hex-6-yl)phenyl]methanesulfonamide

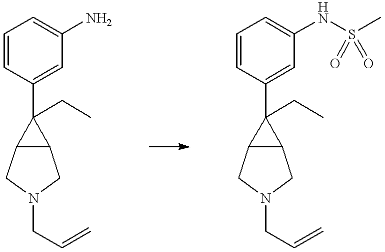

To a solution of 3-(3-allyl-6-ethyl-3-azabicyclo[3.1.0]hex-6-yl)aniline (41 g, 169 mmol) and triethylamine (34 g, 337 mmol) in dichloromethane (750 ml) at -400C was added dropwise methanesulfonyl chloride (23.7 g, 206 mmol) via a dropping funnel. The reaction mixture was a slowly allowed to warm to room temperature over 2 hours and was then stirred at room temperature for 20 hours. The organics were then washed with water (4×500 ml), dried (MgSO₄), filtered and concentrated in vacuo to afford the title compound (59.0 g) as a crude gum.

¹H-NMR (300 MHz, CDCl₃): 0.85 (t, 3H), 1.85 (s, 2H), 1.95 (q, 2H), 2.80-3.20 (m, 9H), 5.10-5.25 (m, 2H), 5.80-5.95 (m, 1 H), 7.00-7.40 (m, 4H).

### PREPARATION 157

N-[3-(6-ethyl-3-azabicyclo[3.1.0]hex-6-yl)phenyl]methanesulfonamide

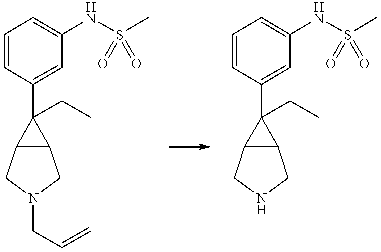

To a degassed solution of N-[3-(3-allyl-6-ethyl-3-azabicyclo[3.1.0]hex-6-yl)phenyl]methanesulfonamide (54.0 g, 169 mmol) and 1,3-dimethylbarbituric acid (80.0 g, 512 mmol) in dichloromethane (500 ml) under a nitrogen atmosphere was added tetrakis(triphenylphosphine)palladium (0) (2.0 g, 1.73 mmol). The mixture was heated at reflux for 8 hours and then stirred at room temperature for 20 hours. The organics were then extracted with 2M HCL (2×100 ml) and water (100 ml). The combined aqueous layers were then washed with dichloromethane (4×100 ml) and freeze dried to give a crude solid. This material was purified by preparative HPLC (condition 4) to afford the trifluoroacetic acid salt of title compound (25.2 g, 53%) as a grey solid.

¹H-NMR (300 MHz, CD₃OD): 0.90 (t, 3H), 1.65 (q, 2H), 2.30-2.40 (m, 2H), 2.90 (s, 3H), 3.25-3.35 (m, 2H), 3.70-3.80 (m, 2H), 7.10-7.15 (m, 2H), 7.20 (s, 1H), 7.30 (t, 1H). MS (AP+): M/Z [MH]+281; C14H20N2O2S+H requires 281.1.

### PREPARATION 158

3-Benzyl-6-methyl-6-(3-nitrophenyl )-3-azabicyclo[3.1.0]hexane-2,4-dione

To a solution of 1-(3-nitrophenyl)-1 -ethanone hydrazone

(100 g, 0.56 mol), in dioxan (1L) was added MnO₂ (350 g, 2.3 mol) and the reaction mixture stirred at room temperature for 30mins. The slurry was filtered through celite and the celite pad washed with dioxan (200mls). The filtrate was returned to a pot and N-benzyl maleimide (110 g,) added portionwise over a period of 20 mins. The reaction mixture was stirred at room temperature for 4 hrs before being heated under reflux for 16 hrs. The reaction mixture was cooled to room temperature and the solvent removed in vacuo. The residue was triturated in methanol (500 mls) and the product isolated by filtration as a white crystalline solid (56%).

NMR (CDCl₃) d: 1.31 (s, 3H), 1.55 (s, 3H), 2.80 (s, 2H), 4.63 (s, 2H), 7.28-7.34 (m, 3H), 7.43-7.45 (d, 2H), 7.52-7.56 (t, 1H), 7.63-7.65 (d, 1H), 8.13-8.16 (d, 1H), 8.17 (s, 1H)

MS (APCI): m/z [MH+] 337.5+H requires 337.3

### PREPARATION 159

6-(3-Aminophenyl)-3-benzyl-6-methyl-3-azabicyclo[3.1.0]hexane-2,4-dione

To a slurry of 3-benzyl-6-methyl-6-(3-nitrophenyl)-3-azabicyclo[3.1.0]hexane-2,4-dione (30 g, 89 mmol) in ethyl acetate (600 mls) was added 5% Pt/C (1.5 g, 5 wt %). The mixture was hydrogenated at 4 atm. (=60 p.s.i.)/room temperature for 18 hrs. The slurry was filtered through arbacel and the resulting solution evaporated in vacuo to yield the product as a white crystalline solid (24 g, 88%).

NMR (CDCl3) d: 1.26 (s, 3H), 2.74 (s, 2H), 3.7 (2H, bs), 4.60 (s, 2H), 6.56-6.58 (d, 1H), 6.60 (s, 1H), 6.65-6.67 (d, 1H), 7.07-7.11 (t, 1H), 7.26-7.33 (m, 3H), 7.42-7.44 (m, 2H).

MS (APCI): m/z [MH+] 307.5+H requires 307.4

### PREPARATION 160

### N-{3-[3-Benzyl-6-methyl-2,4-dioxo-3-azabicyclo[3.1.0]hex-6-yl]phenyl}methanesulfonamide

To a solution of 6-(3-aminophenyl)-3-benzyl-6-methyl-3-azabicyclo[3.1.0]hexane-2,4-dione (24 g, 78 mmol) in ethyl acetate (480mls) was added pyridine (9.5mls, 118 mmol) followed by the slow addition of methane sulfonyl chloride (9.1mIs, 118 mmol). The reaction was stirred at room temperature for 2.5 hrs. The reaction mixture was washed sequentially with 1M HCL solution (120mls) and water (120mls). The ethyl acetate was dried over MgSO₄ and evaporated in vacuo to yield the product as an orange solid (30 g, 99%).

NMR (CDCl3) d: 1.27 (s, 3H), 2.77 (s, 2H), 3.02 (s, 3H), 4.61 (s, 2H), 7.08-7.14 (m, 3H), 7.26-7.32 (m, 4H), 7.41-7.42 (d, 2H).

MS (APCI): m/z [MH+] 385.7+H requires 385.5

### PREPARATION 161

N-{3-Benzyl-6-methyl-3-azabiyclo[3.1.0]hex-6-yl]phenyl} methanesulfonamide

To a solution of N-{3-[3-benzyl-6-methyl-2,4-dioxo-3-azabicyclo[3. I.0]hex-6-yl]phenyl}methanesulfonamide (1509, 391 mmol), under nitrogen was added sodium borohydride (31 g, 820 mmol). The reaction mixture was cooled to <10° C. and the BF₃.OEt₂ (138.6mls, 1094 mmol) added dropwise maintaining the temperature at <10° C. The reaction mixture was allowed to warm to room temperature over 2 hrs before being heated under reflux for a further 8.5 hrs. The reaction mixture was cooled to between 0° C. and 50C and an aqueous solution of piperazine (198.5 g, 2304 mmol in 1.26L of water) added. The reaction mixture was then heated under reflux for a period of 18 hrs. The THF was removed under vacuum, ethyl acetate (900 mls) added, and the phases were separated. The aqueous phase was extracted with a second portion of ethyl acetate (450mls). The organic phases were combined and washed with water (750 mls). The organics were dried over MgSO₄ and evaporated in vacuo to yield the product as a white crystalline solid (129 g, 93%).

NMR (CDCl3) d: 2.62 (s, 3H), 2.80-2.83 (d, 2H), 2.99 (s, 3H), 3.03-3.07 (d, 2H), 3.68 (s, 2H), 7.01-7.02 (s, 1 H), 7.06-7.08 (m, 2H), 7.22-7.26 (m, 3H), 7.30-7.32 (m, 3H).

MS (APCI): m/z [MH+]357.5+H requires 357.5

### PREPARATION 162

### N-(3-[6-methyl-3-azabicyclo[3.1.0]hex-6-yl]phenyl}methanesulfonamide

To a solution of N-{3-benzyl-6-methyl-3-azabiyclo[3.1.0]hex-6-yl]phenyl} methanesulfonamide (20 g, 56 mmol), in methanol, was added ammonium formate (10.6 g, 168 mmol) and the reaction stirred for 5 minutes. 10% Pd/C (8 g) was added and the resulting mixture heated at reflux for 16 hrs. The mixture was allowed to cool and the catalyst removed by filtration through celite. The solvent was removed in vacuo to yield the product as a pale yellow oil, which solidified on standing (15.2 g, 85%).

NMR (CDCl3) d: 1.27 (s, 3H), 1.85-1.88 (d, 2H), 2.93 (s, 3H), 3.07-3.10 (d, 2H), 3.39-3.44 (d, 2H), 6.92-6.97 (m, 2H), 7.06 (s, 1 H), 7.20-7.23 (m, 1H).

MS (APCI): m/z [MH+]267.4+H requires 267.3

### PREPARATION 163

### 3-Benzyl-6-ethyl-6-(3-nitrophenyl)-3-azabicyclo[3.1.0]hexane-2,4-dione

To a solution of 1-(3-nitrophenyl)-1-propanone hydrazone (42.1 gg, 217 mmol), in dioxan (630 mls) was added MnO₂ (126 g, 1440 mmol) and the reaction mixture stirred at room temperature for 20 mins. The slurry was filtered through celite and the celite pad washed with dioxan (200mis). The filtrate was returned to a pot and N-benzyl maleimide (44.9 g, 239 mmol) added portionwise over a period of 20 mins. The reaction mixture was stirred at room temperature for 60 hrs before being heated under reflux for 16 hrs. The reaction mixture was cooled to room temperature and the solvent removed in vacuo. The residue was heated to reflux in methanol (1200 mls) for 3 hours and then cooled to room temperature. The product was isolated by filtration as a white crystalline solid (42.4 g, 56%).

NMR (CDCl3) d: 0.69-0.73 (t, 3H), 1.47-1.49 (q, 2H), 2.78 (s, 2H), 4.64 (s, 2H), 7.3-7.32 (m, 2H), 7.43-7.44 (d, 1H), 7.52-7.55 (t, 1H), 7.62-7.65 (d, 2H), 8.17-8.18 (m, 3H).

MS (APCI): m/z [MH+] 351.5+H requires 351.3

### PREPARATION 164

### 6-(3-Aminophenyl)-3-benzyl-6-ethyl-3-azabicyclo[3.1.0]hexane-2,4-dione

To a slurry of 3-benzyl-6-ethyl-6-(3-nitrophenyl)-3-azabicyclo[3.1.0]hexane-2,4-dione (42.1 g, 120 mmol) in ethyl acetate (850 mls) was added 5% Pt/C (2.1 g, 5wt% ). The mixture was hydrogenated at 60psi/ room temperature for 18 hrs. The slurry was filtered through arbacel and the resulting solution evaporated in vacuo to yield the product as a white crystalline solid (34.1 g, 89% ).

NMR (CDCl3) d: 0.70-0.74 (t, 3H), 1.41-1.47 (q, 2H), 2.73 (s, 2H), 3.68 (bs, 2H), 4.61 (s, 2H), 6.55-6.57 (d, 1H), 6.60 (s, 1H), 6.66-6.68 (d, 1H), 7.07-7.10 (t, 1H), 7.28-7.32 (m, 3H), 7.41-7.43 (d, 2H).

MS (APCI): m/z [MH+] 321.4+H requires 321.4

### PREPARATION 165

### N-{3-[3-Benzyl-6-ethyl-2,4-dioxo-3-azabicyclo[3.1.0]hex-6-yl]phenyl}methanesulfonamide

To a solution of 6-(3-aminophenyl)-3-benzyl-6-ethyl-3-azabicyclo[3.1.0]hexane-2,4-dione (31.5 g, 98 mmol) in dichloromethane (250 mls) was added pyridine (9.5 mls, 118 mmol) followed by the slow addition of methane sulfonyl chloride (9.1mIs, 118 mmol). The reaction was stirred at room temperature for 16 hrs. The reaction mixture was washed sequentially with 1M HCL solution (250mis) and water (120 mls). The dichloromethane was dried over MgSO₄ and evaporated in vacuo to yield the product as a waxy pink solid (38.2 g, 98% ).

NMR (CDCl3) d: 0.68-0.72 (t, 3H), 1.42-1.47 (q, 2H), 2.75 (s, 2H), 3.02 (s, 3H), 4.62 (s, 2H), 7.13-7.18 (m, 3H), 7.29-7.42 (m, 4H), 7.41-7.43 (d, 2H).

MS (APCI): m/z [MH+] 399.6 +H requires 399.5

### PREPARATION 166

### N-{3-Benzyl-6-ethyl-3-azabicyclo[3.1.0]hex-6-yl]phenyl} methanesulfonamide

To a solution of N-{3-[3-benzyl-6-ethyl-2,4-dioxo-3-azabicyclo[3.1.0]hex-6-yl]phenyl}methanesulfonamide (38.2 g, 95 mmol), in THF (200 mls) under nitrogen was added sodium borohydride (7.46 g, 201 mmol). The reaction mixture was cooled to <10□C and the BF₃.OEt₂ (38.lmls, 268 mmol) added dropwise maintaining the temperature at <10□C. The reaction mixture was allowed to warm to room temperature over 2 hrs before being heated under reflux for a further 12 hrs. The reaction mixture was cooled to between 0° C. and 5° C. and an aqueous solution of piperazine (48.7 g, 565 mmol in 320 mls of water) added. The reaction mixture was then heated under reflux for a period of 18 hrs. The THF was removed under vacuum, ethyl acetate (200 mls) added, and the phases were separated. The aqueous phase was extracted with a second portion of ethyl acetate (200 mls). The organic phases were combined and washed with 3 separate portions of water (3×400 mls). The organics were dried over MgSO₄ and evaporated in vacuo to yield the product as a white crystalline solid (33.5 g, 94% ).

NMR (CDCl3) d: 0.84-0.88 (t, 3H), 1.76-1.77 (d, 2H), 2.06-2.12 (q, 2H), 2.79-2.81 (d, 2H), 2.99 (s, 3H), 3.06-3.08 (d, 2H), 3.67 (s, 2H), 7.01-7.03 (d, 1H), 7.08-7.10 (d, 2H), 7.22-7.26 (m, 3H), 7.30-7.32 (m, 3H).

MS (APCI): m/z [MH+] 371.3+H requires 371.5

### PREPARATION 167

### N-{3-[6-Ethyl-3-azabicyclo[3.1.0]hex-6-yl]phenyl}methanesulfonamide

To a solution of N-{3-benzyl-6-ethyl-3-azabiyclo[3.1.0]hex-6-yl]phenyl} methanesulfonamide (500mg, 1.34 mmol), in methanol (30 mls), was added ammonium formate (255mg, 4.05 mmol) and the reaction stirred for 5 minutes. 10% Pd/C (200 mg) was added and the resulting mixture heated at reflux for 2 hrs. The mixture was allowed to cool and the catalyst removed by filtration through celite. The solvent was removed in vacuo to yield the product as a pale yellow oil, which solidified on standing (1 5.2 g, 85% ).

NMR (CDCl3) d: 0.80-0.84 (t, 3H), 1.64-1.69 (q, 3H), 1.82-1.86 (d, 2H), 2.98 (s, 3H), 3.12-3.18 (d, 2H), 3.21-3.26 (d, 2H), 7.01-7.06 (d, 1H), 7.10-7.14 (m, 2H), 7.25-7.28 (m, 1H).

MS (APCI): m/z [MH+] 281.7 +H requires 281.4

Other building block materials useful in synthesising compounds of formula (I) with various different R⁴ groups are available from the sources indicated in the table below, and routine derivatisation thereof, or analogy synthesis.

| | | |
|---|---|---|
| | Example of | |
| R⁴ Substructure | commercial source | Literature reference |

—(CH₂)nO(CH₂)nR
examples

| | | |
|---|---|---|
| 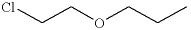 | ALDRICH | |
| 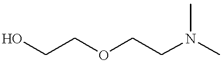 | ALDRICH | |
| 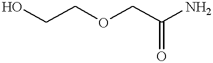 | SALOR | |
| 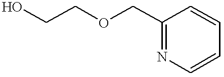 | | 1. Org.Magn.Reson., 1975, Vol 7, 488-495. |
| 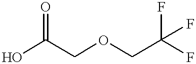 | | WO 8707270 Alcohol US 5157159 |
| 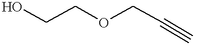 | MAYBRIDGE | |
| 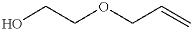 | ALDRICH | |
| 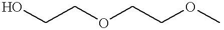 | ALDRICH | |
| 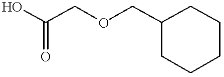 | | Bull. Soc. Chim. Fr.; 1947, 616. Alcohol-EP-0811621 |
| (CH₂)nO(CH₂)nAryl examples | | |
| 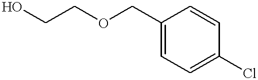 | BIONET | |
| 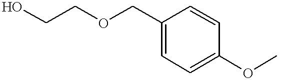 | | 1. J. Org. Chem., 1987, 52 (12), 2594. 2. Org. Prep. Proceed. Int; 23, 4; 1991, 427. |
| All other (CH2)nO(CH2)nAryl | | For a general |
| examples could be prepared | | procedure see; J. Am. |
| via 2 step process from a | | Chem. Soc., 1951, 3159- |
| benzyl alcohol or benzyl | | 3162. |
| chloride | | |
| Aryl substituents on R⁴ (mix. | | |
| of aryl and aryloxy examples): | | |
| 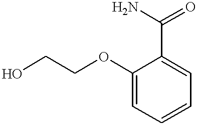 | SALOR | J. Med. Chem., 1985, 28, 1427. |
| 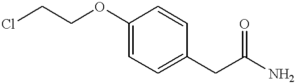 | | J. Med. Chem., EN; 28, 10, 1985, 1427. |
| 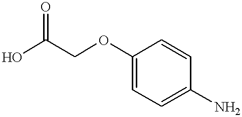 | SALOR | |
| 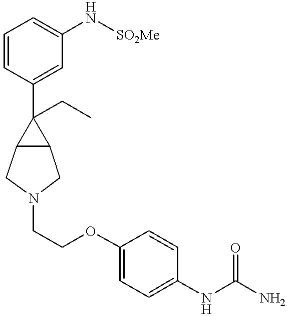 | By manipulation of the product above. | |
| | | DE 2135678; |
| | | DE 3636333 |
| 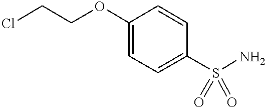 | | Rev. Med.-Chiv., 1985, 89 (2), 316-20. |
| 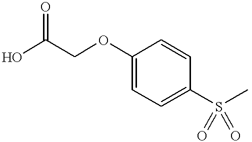 | SPECS | |
| 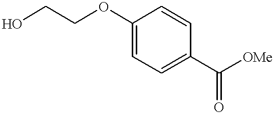 | APIN | J. Med. Chem., 28, 10, 1985, 1427 |
| 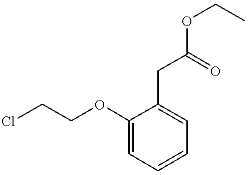 | | J. Med. Chem., 28, 10, 1985, 1427 |
| 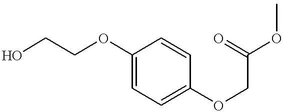 | | EP-0171760 J. Med. Chem., 28, 10, 1985, 1427. |
| 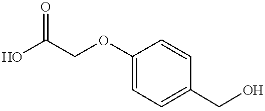 | LANCASTER | |
| 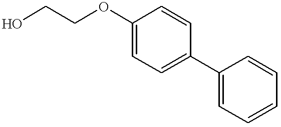 | ICN-RF | |
| 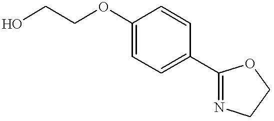 | MAYBRIDGE | |
| 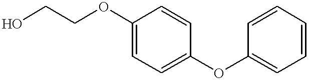 | SALOR | |
| 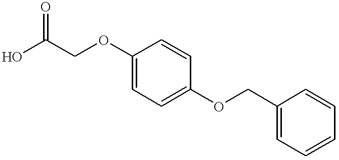 | MAYBRIDGE | |
| 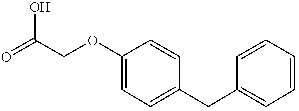 | | Agric. Biol. Chem., 1978, 1767. WO 9611192 (alcohol) WO 9610999 Imidazole analogue: J. Med Chem., 1981, 24(10), 1139 US-4713387 |
| 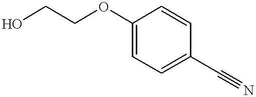 | MAYBRIDGE | J. Med. Chem., 28, 10, 1985, 1427. |
| Substituents of the basic R⁴ | | |
| alkyl/alkenyl/alkynyl chains: | | |
| 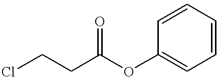 | ICN-RF | |
| 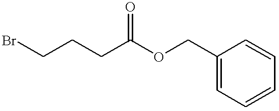 | WYCHEM | |
| 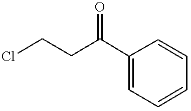 | ALDRICH | |
| 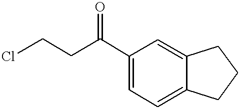 | SALOR | |
| 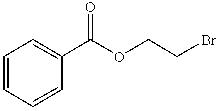 | ALDRICH | |
| 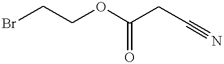 | | J. Am. Chem. Soc., 78, 1956, 4944. Alcohol: EP-136260 |
| 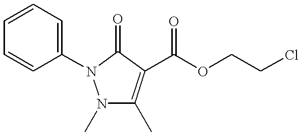 | SALOR | |
| 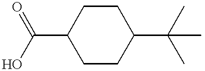 | ALDRICH | |
| 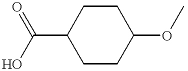 | ALDRICH | |
| 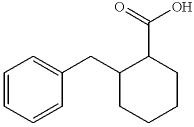 | SALOR | |
| 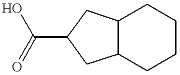 | | J. Org. Chem., 1954, 1449. |
| 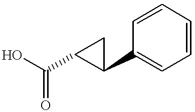 | ALDRICH | |
| 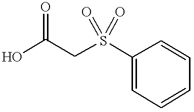 | LANCASTER Alcohol-ALDRICH | |
| 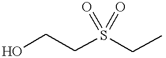 | LANCASTER | |
| 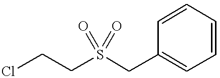 | LANCASTER | |
| 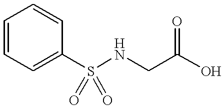 | SPECS | |
| 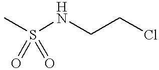 | MDA | |
| 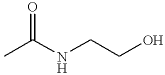 | ALDRICH | |
| 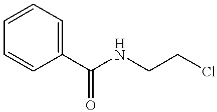 | ALDRICH MAYBRIDGE | |
| 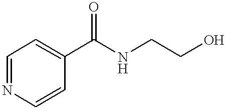 | ALDRICH | |
| 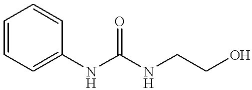 | MAYBRIDGE | |
| 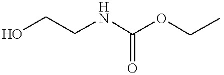 | SALOR | |
| 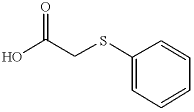 | ALDRICH | |
| 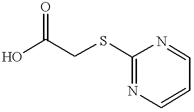 | ALDRICH | |

1. A substance which is a compound of formula I, 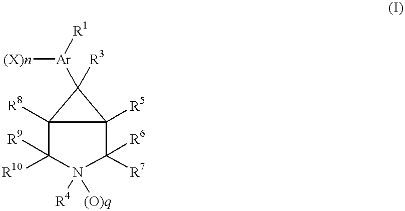
wherein the “Ar” ring represents an optionally benzo-fused phenyl or 5- or 6-membered heteroaryl ring;
R¹ when taken alone is H, halogen, NO₂, NH₂, NY²WY¹, Het¹, AD, CO₂R⁷, C(O)R⁸, C(═NOH)R⁸, or OE,
Y² is H, C₁₋₆ alkyl, C₃₋₆ alkenyl (each of which alkyl and alkenyl is optionally substituted by aryl, aryloxy or Het¹),
W is SO₂, CO, C(O)O, P(Y¹)═O, P(Y¹)═S,
Y¹ is C₁₋₁₀ alkyl (optionally substituted by one or more substituents independently selected from halogen, OH, C₁₋₄ alkoxy, C₁₋₆ alkanoyloxy, CONH₂, C₁₋₄ alkoxycarbonyl, NH₂, aryl, mono- or di(C₁₋₄ alkyl)amino, C₃₋₈ cycloalkyl, phthalimidyl, Het), Het¹, aryl (optionally substituted by one or more substituents independently selected from C₁₋₄ alkyl, C₁₋₄ haloalkyl and halogen), NH₂, N(C₁₋₆alkyl)₂ or NH(C₁₋₄ alkyl),
Het¹ is a heterocyclic group containing up to 4 heteroatoms selected from N, O and S, which may comprise up to 3 rings (preferably a heteroaryl group,
optionally benzo- or pyrido-fused heteroaryl),
optionally substituted by one or more substituents independently selected from C₁₋₆ alkyl, C₁₋₆ alkoxy, C₃₋₆ cycloalkyl, C₁₋₆ haloalkoxy, C₁₋₆haloalkyl, C₃₋₆ halocycloalkyl, ═O, OH, halogen, NO₂, SiR^{19a}R^{19b}R^{19c}, CON^{20a}R^{20b}, NR^{20a}R^{20b}, SR^{21a}, NR^{21b}SO₂R^{22a}, NR^{21c}C(O)OR^{22b}, NR^{21d}COR^{22c}, and C₁₋₆ alkoxycarbonyl,
and if a S atom is present in a ring, it can be present as part of a —S—, S(O)— or —S(O₂)— group, and carbon atoms in the ring can be present as a part of a carbonyl moiety;
R^{19a}, R^{19b}, R^{19c} each independently represent C₁₋₆ alkyl or aryl,
R^{20a} and R^{20b} each independently represent H, C₁₋₆ alkyl, aryl, (C₁₋₄ alkyl)phenyl, each of which alkyl, aryl and alkylphenyl are optionally substituted by one or more C₁₋₄ alkyl, C₁₋₄ alkoxy, OH, NO₂, NH₂ and/or halogen,
or R^{20a} and R^{20b} can be taken together with the N atom to which they are attached, to form a 4- to 6-membered ring optionally substituted by one or more substitutuents independently selected from one or more C₁₋₄ alkyl, C₁₋₄ alkoxy, OH, =O, NO₂, NH₂ and/or halogen,
R^{21a, b, c} and deach independently represent H, C_{1 6} alkyl, aryl or C₁₋₄ alkylphenyl, each of which alkyl, aryl, and alkylphenyl are optionally substituted by one or more C₁₋₄ alkyl, C₁₋₄ alkoxy, OH, NO₂, halogen, NH₂,
R^{22a, b and c} each independently represent C₁₋₆ alkyl, aryl or C₁₋₄ alkylphenyl, each of which alkyl, aryl, and alkylphenyl are optionally substituted by one or more C₁₋₄ alkyl, C₁₋₄ alkoxy, OH, NO₂, halogen, NH₂,
A is C₁₋₄ alkylene, C₂₋₄ alkenylene or C₂₋₄ alkynylene, each of which is optionally substituted by one or more C₁₋₄ alkyl, C₁₋₄ alkoxy, halogen and/or OH,
D is H, OH, CN, NR²⁵R²⁶, CONR²⁵R²⁶ NHR²⁷ CO₂R²⁸, COR²⁹, C(═NOH)R²⁹
or AD is CN, NR²⁵R²⁶, CONR²⁵R²⁶,
where R²⁵ and R²⁶ are either each independently H, C₁₋₃ alkyl, C₃₋₈ cycloalkyl, aryl, C₁₋₄ alkylphenyl (each of which C₁₋₃ alkyl, C₃₋₈ cycloalkyl, aryl and C₁₋₄ alkylphenyl are optionally substituted by one or more NO₂, halogen, C₁₋₄ alkyl and/or C₁₋₄ alkoxy, (each of which latter C₁₋₄ alkyl and C₁₋₄ alkoxy is optionally substituted by one or more halogen)),
or R²⁵ and R²⁶ are taken together with the N atom to which they are attached and can form a 4- to 7-membered heterocyclic ring optionally incorporating one or more further hetero atoms selected from N, O and S, and which ring is optionally substituted by one or more C₁₋₄ alkyl, OH, ═O, NO₂, NH₂ and/or halogen,
R²⁷ is COR³⁰, CO₂R^{31a}, SO₂R^{31b},
R²⁸ and R²⁹ are each independently H, C₁₋₆ alkyl, C₃₋₈ cycloalkyl, aryl or C₁₋₄alkylphenyl, each of which C₁₋₆ alkyl, C₃₋₈ cycloalkyl, aryl and C₁₋₄ alkylphenyl are optionally substituted by one or more NO₂, halogen, C₁₋₄ alkyl, C₁₋₄ alkoxy (each of which latter C₁₋₄ alkyl and C₁₋₄ alkoxy are optionally substituted by one or more halogen),
R³⁰ is H, C₁₋₄ alkyl, C₃₋₈ cycloalkyl, C₁₋₄ alkoxy, C₃₋₈ cycloalkyloxy, aryl, aryloxy, C₁₋₄ alkylphenyl, phenyl(C₁₋₄ )alkoxy, (each of which C₁₋₄ alkyl, C₃₋₈ cycloalkyl, C₁₋₄ alkoxy, C₃₋₈ cycloalkyloxy, aryl, aryloxy, C₁₋₄ alkylphenyl and phenyl(C₁₋₄)alkoxy are optionally substituted by one or more NO₂, halogen, C₁₋₄ alkyl, C₁₋₄ alkoxy (which latter alkyl and alkoxy are optionally substituted by one or more halogen)),
R^{31a} and R^{31b} are each independently C₁₋₄ alkyl, C cycloalkyl, aryl or C₁₋₄ alkylphenyl, each of which is optionally substituted by one or more NO₂, halogen, C₁₋₄ alkyl or C₁₋₄ alkoxy, each of which latter alkyl and alkoxy is optionally substituted by one more halogen
E is H, CONR³²R³³, CSNR³²R³³, COR³⁴, CO₂R³⁴, COCH(R^{34a})NH₂, R³⁵, CH₂CO₂R^{35a}, CHR^{35b}CO₂R^{35a}, CH₂OCO₂R^{35c}, CHR^{35d}OCO₂R^{35c}, COCR³⁶═CR³⁷NH₂, COCHR³⁶CHR³⁷NH₂, or PO(OR³⁸)₂,
R³² and R³³ are each independently H, C₃₋₁₀ alkylalkenyl, C₃₇ cycloalkyl (optionally substituted by C₁₋₄ alkyl), phenyl (optionally substituted by (X)ₙ), C₁₋₁₀ alkyl (optionally substituted by C₄₋₇ cycloalkyl (optionally substituted by C₁₋₄ alkyl) or phenyl optionally substituted by (X)ₙ),
or R³² and R³³ can be taken together with the N atom to which they are attached and can form a 5- to 8-membered heterocycle optionally comprising further hetero atoms selected from N, O and S, which heterocycle is optionally substituted by C₁₋₄ alkyl, optionally substituted by one or more halogen,
R³⁴ is H, C₄₋₇ cycloalkyl (optionally substituted by one or more C₁₋₄ alkyl), phenyl (optionally substituted by (X)ₙ, C₁₋₄ alkanoyloxy, NR³²R³³, CONR³²R³³ and/or OH), or C₁₋₆ alkyl (optionally substituted by one or more halogen, C₄₋₇ cycloalkyl (optionally substituted by one or more C₁₋₄ alkyl), or phenyl (optionally substituted by (X)ₙ,C₁₋₄ alkanoyloxy, NR³²R³³, CONR³²R³³ and/or OH)),
R^{34a} is H, C₁₋₆ alkyl (optionally substituted by one or more halogen, C₄₋₇ cycloalkyl (optionally substituted by one or more C₁₋₄ alkyl), or phenyl (optionally substituted by (X)ₙ, C₁₋₄ alkanoyloxy, NR³²R³³, CONR³²R³³ and/or OH)), C₄₋₇ cycloalkyl (optionally substituted by one or more C₁₋₄ alkyl), phenyl (optionally substituted by (X)ₙ,C₁₋₄ alkanoyloxy, NR³²R³³, CONR³²R³³ and/or OH) or a naturally occuring amino acid substituent,
R³⁵ is C₄₋₇ cycloalkyl optionally substituted by one or more C₁₋₄ alkyl, phenyl (optionally substituted by one or more (X)ₙ, C₁₋₄ alkanoyl, NHR³², CON(R³²)₂, and/or OH), C₁₋₆ alkyl (optionally substituted by C₄₋₇ cycloalkyl optionally substituted by one or more C₁₋₄ alkyl, or phenyl (optionally substituted by one or more (X)ₙ, C₁₋₄ alkanoyl, NHR³², CON(R³²)₂, and/or OH)), C₁₋₄ alkoxy(C₁₋₄ alkyl), phenyl(C₁₋₄)alkyloxy(C₁₋₄)alkyl, tetrahydropyranyl, tetrahydrofuranyl, cinnamyl or trimethylsilyl,
R^{35a,b,c and d} are each independently H, C₄₋₇ cycloalkyl optionally substituted by one or more C₁₋₄ alkyl, phenyl optionally substituted by one or more (X), or C₁₋₆ alkyl (optionally substituted by C₄₋₇ cycloalkyl optionally substituted by one or more C₁₋₄ alkyl, or phenyl optionally substituted by one or more (X)ₙ),
R³⁶ and R³⁷ each independently represent H, C₃₋₆ alkylalkenyl, C₄₋₇ cycloalkyl, phenyl optionally substituted by one or more (X)ₙ, or C₁₋₆ alkyl (optionally substituted by C₄₋₇ cycloalkyl optionally substituted by one or more C₁₋₄ alkyl, or phenyl optionally substituted by one or more (X)ₙ),
R³⁸ is C₄₋₇ cycloalkyl optionally substituted by one or more C₁₋₄ alkyl, phenyl optionally substituted by one or more (X)ₙ, or C₁₋₆ alkyl (optionally substituted by C₄₋₇ cycloalkyl optionally substituted by one or more C₁₋₄ alkyl, or phenyl optionally substituted by one or more (X)ₙ),
R² when taken alone is H or halogen;
or R¹ and R², when attached to adjacent carbon atoms, can be taken together with the carbon atoms to which they are attached, and may represent Het^{1a};
Het^{1a} is a heterocyclic group containing up to 4 heteroatoms selected from N, O and S, which may comprise up to 3 rings (and is preferably an optionally benzo-fused 5- to 7-membered heterocyclic ring) and which group is optionally substituted by one or more substituents independently selected from OH, ═O, halogen, C₁₋₄ alkyl, C₁₋₄ haloalkyl, C₁₋₄ alkoxy and C₁₋₄ haloalkoxy,
which C₁₋₄ alkyl, C₁₋₄ haloalkyl, C₁₋₄ alkoxy and C₁₋₄ haloalkoxy groups can be optionally substituted by one or more C₃₋₆ cycloalkyl, aryl(C₁₋₆)alkyl, which aryl group is optionally substituted by one or more halogen, C₁₋₄ alkyl, C₁₋₄ haloalkyl, C₁₋₄ alkoxy and C₁₋₄ haloalkoxy,
which latter C₁₋₄ alkyl, C₁₋₄ haloalkyl, C₁₋₄ alkoxy and C₁₋₄ haloalkoxy groups can be optionally substituted by one or more NR²³R²⁴, NR²³S(O)ₙR²⁴, NR²³C(O)ₘR²⁴,
and if a S atom is present in a ring, it can be present as part of a —S—, S(O)— or —S(O₂)— group,
which R²³ and R²⁴ when taken alone independently represent H, C₁₋₄ alkyl, or C₁₋₄ haloalkyl,
or R²³ and R²⁴ can be taken together with the N atom to which they are attached, to form a 4- to 6-membered heterocyclic ring optionally comprising one or more further heteroatoms selected from, N, O, or S, and which heterocyclic ring is optionally substituted by one or more halogen, C₁₋₄ alkyl, C₁₋₄ haloalkyl, C₁₋₄ alkoxy and/or C₁₋₄ haloalkoxy groups,
R³ is H, CN, halogen, C₁₋₄ alkoxy, C₁₋₄ alkoxycarbonyl, C₂ alkanoyl, C₂₋₆ alkanoyloxy, C₃₋₈ cycloalkyl, C₃₋₈ cycloalkyloxy, C₄₉ cycloalkanoyl, aryl, aryloxy, heteroaryl, saturated heterocycle, NR¹²R¹³, CONR¹²R¹³, NY²WY¹, C₁₋₆ alkyl, C₂₋₁₀ alkenyl, C₂₋₁₀ alkynyl, (each of which alkyl, alkenyl and alkynyl groups is optionally substituted by one or more CN, halogen, OH, C₁₋₆ alkoxy, C₁₋₆ alkoxycarbonyl, C₂₋₆ alkyloxycarbonyloxy, C₁₋₆ alkanoyl, C₁₋₆ alkanoyloxy, C₃₋₈ cycloalkyl, C₃₋₈ cycloalkyloxy, C₄₋₉ cycloalkanoyl, aryl, aryloxy, heteroaryl, saturated heterocycle, NR¹²R¹³, CONR¹²R¹³ and/or NY²WY¹),
R⁴ is C₁₋₁₀ alkyl, C₃₋₁₀ alkenyl or C₃₋₁₀ alkynyl, each of which groups is linked to the N atom via a sp3 carbon, and which group is substituted by one or more substituents selected from:
C₂₋₆ alkoxy [substituted by one or more groups selected from OH, NR²⁵R²⁶, CONR²⁵R²⁶, halogen, C₁₋₆ alkoxy, C₂₋₄ alkynyl, C₂₋₄ alkenyl, heteroaryl¹, aryl¹, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O),(C₁alkyl), S(O),(aryl¹), S(O),(heteroaryl¹), SO₂NR²⁵R²⁶ and cycloalkyl¹],
S(O)ₙC₁₋₆alkyl [optionally substituted by one or more groups selected from OH, NR²⁵R²⁶, CONR²⁵R²⁶, halogen, C₁₋₆ alkoxy, C₂₋₄ alkynyl, C₂₋₄ alkenyl, heteroaryl¹, aryl¹, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O)ₙ(C₁₋₆ alkyl), S(O)ₙ(aryl¹), S(O)ₙ(heteroaryl¹), SO₂NR²⁵R²⁶ and cycloalkyl¹],
aryl²,
CO₂CH₂(heteroaryl¹),
CO₂CH₂(aryl¹),
cycloalkyl¹,
CO(heteroaryl¹),
CO(aryl¹),
OCO(aryl¹),
OCO(heteroaryl¹),
OCO(C₁alkyl),
OCOCH₂CN,
CO₂(heteroaryl¹),
CO₂(aryl¹),
COCH₂(heteroaryl¹),
S(O)ₙCH₂aryl¹,
S(O)ₙ(heteroaryl¹),
S(O)ₙCH₂(heteroaryl¹),
NHSO₂aryl¹,
NHSO₂(C₁₋₆ alkyl),
NHSO₂(heteroaryl¹),
NHSO₂CH₂(heteroaryl¹),
NHSO₂CH₂(aryl¹),
NHCOaryl¹,
NHCO(C₁alkyl),
NHCONHaryl¹,
NHCONH(C₁₋₆ alkyl),
NHCOheteroaryl¹,
NHCONHheteroaryl¹,
NHCO₂(aryl¹),
NHCO₂(C₁₋₆ alkyl),
NHCO₂(heteroaryl¹),
aryl²oxy,
heteroaryl¹oxy,
C₁₋₄ alkoxycarbonyl substituted by C₁₋₆ alkyl, aryl, C₁₋₆ alkoxy, CH₂(aryl¹), C₁₋₄ haloalkyl, halogen, OH, CN or NR²⁵R²⁵,
C₂₋₆ alkanoyl substituted by C₁₋₄ alkyl, aryl, C₁₋₄ alkoxy, CH₂(aryl¹), C₁₋₄ haloalkyl, halogen, OH, CN or NR²⁵R²⁶,
C₂₋₆ alkanoyloxy substituted by C₁₋₆ alkyl, aryl, C₁₋₄ alkoxy, CH₂(aryl¹), C₁₋₄ haloalkyl, halogen, OH, CN or NR²⁵R²⁶,
cycloalkyl¹oxy,
COcycloalkyl¹,
heterocycle substituted by one or more substituent selected from C₁₋₆ alkyl(substituted by OH), CONR²⁵R²⁶₁ CH₂CONR R , NRR26, NHCONR²⁵R²⁶ alkyl), SO₂NR²⁵R²⁶, SO₂(C₁₋₆ alkyl), CO₂(C₁₋₆ alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl, heterocyclyloxy substituted by one or more substituent selected from C₁₋₆ alkyl(substituted by OH), CONR²⁵R²⁶, CH₂CONR²⁵R²⁶, NR²⁵R²⁶ NHCONR²⁵R²⁶, CO(C₁₋₆ alkyl), SO₂NR²⁵R²⁶, SO₂(Cl alkyl), CO₂(Cl alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl,
WHEREIN aryl¹ is phenyl optionally fused to a C₅₇ carbocyclic ring, which group is optionally substituted by one or more substituent selected from CL-₆ alkyl(optionally substituted by OH, CN or halogen), C₁₋₆ haloalkoxy, OH, ═O, NY²WY¹, halogen, C₁₋₆ alkoxy, CONR²⁵R²⁶ CH₂CONR²⁵R²⁶, NR²⁵R²⁶, NHCONR²⁵R²⁶, CO(C₁₋₆ alkyl), COaryl, COheteroaryl, SO₂NR²⁵R²⁶, S(O)ₙ(C₁₋₄ alkyl), S(O)ₙ(aryl), S(O)ₙ(heteroaryl), CO₂(C₁₋₆ alkyl), CO₂(aryl), CO₂(heteroaryl), CO₂H, (CH₂)₁₋₄CO₂(C₁alkyl), (CH₂)₁₋₄CO₂H, (CH₂)₁₋₄CO₂(aryl), (CH₂)₁₋₄ CO₂(heteroaryl), O(CH₂)₁₋₄CO₂(C₁alkyl), O(CH₂)₁₋₄CO₂H, O(CH₂)₁4CO₂(aryl), O(CH₂)₁₋₄ CO₂(heteroaryl), aryl, heterocyclyl, aryloxy, aryl(CH₂)oxy, aryl(CH₂), CN, O(CH₂)₁₋₄ CONR²⁵R²⁶ and C₃₋₇ cycloalkyl,
aryl² is phenyl optionally fused to a C₅₋₇ carbocyclic ring, which group is substituted by one or more substituent selected from C₁₋₆ alkyl(substituted by OH), CONR²⁵R²⁶, CH₂CONR²⁵R²⁶, NHCONR²⁵R²⁶, CO(C₁₋₆ alkyl), COaryl, COheteroaryl, SO₂NR²⁵R²⁶, S(O)ₙ(C₁alkyl), S(O).(aryl), S(O),(heteroaryl), CO₂(C₁₋₆ alkyl), CO₂(aryl), CO₂(heteroaryl), CO₂H, (CH₂)₁₋₄CO₂(C₁₋₆ alkyl), (CH₂)₁₋₄CO₂H, (CH₂)₁₋₄CO₂(aryl), (CH₂)₁₋₄CO₂(heteroaryl), O(CH₂)₁₋₄CO₂(Cl alkyl), O(CH₂)₁₋₄CO₂H, O(CH₂)₁₋₄CO₂(aryl), O(CH₂)₁₋₄CO₂(heteroaryl), aryl, heterocyclyl, aryloxy, aryl(CH₂)oxy, aryl(CH₂), CN, O(CH₂)₁₋₄CONR 25R²⁶ and C₃₋₇ cycloalkyl, heteroaryl¹ is heteroaryl optionally fused to a C₅₋₇ carbocyclic ring, which group is optionally substituted by one or more substituent selected from C₁₋₆ alkyl(optionally substituted by OH, CN or halogen), C₁₋₄ haloalkoxy, OH, ═O, NY²WY , halogen, C₁₋₆ alkoxy, CONR²⁵R²⁶, CH₂CONR²⁵R²⁶, NR²⁵R²⁶, NHCONR²⁵R²⁶, CO(C₁₋₆ alkyl), COaryl, Coheteroaryl, SO₂NR²⁵R²⁶, S(O)ₙ(C₁₋₆ alkyl), S(O)ₙ(aryl), S(O)ₙ(heteroaryl), CO₂(C₁₋₆ alkyl), CO₂(aryl), CO₂(heteroaryl), CO₂H, (CH₂)₁₋₄CO₂(C₁₋₆ alkyl), (CH₂)₁₋₄CO₂H, (CH₂)₁₋₄CO₂(aryl), (CH₂)₁₋₄CO₂(heteroaryl), O(CH₂)₁₋₄CO₂(C₁alkyl), O(CH₂)₁₋₄CO₂H, O(CH₂)₁₋₄CO₂(aryl), O(CH₂)₁₋₄CO₂(heteroaryl), aryl, heterocyclyl, aryloxy, aryl(CH₂)oxy, aryl(CH₂), CN, O(CH₂)₁₄CONR²⁵R²⁶ and C₃₋₇ cycloalkyl,
cycloalkyl¹ is a C₃₋₁₀ carbocyclic system with one or two rings and which is substituted by C₁₋₆ alkyl, aryl, C₁₋₆ alkoxy, CH₂(aryl¹), C₁₋₄ haloalkyl, halogen, OH, CN or NR²⁵R²⁶,
WITH THE PROVISO THAT THERE ARE NO N-R4 GROUPS WHEREIN THERE IS A HETERO-ATOM LINKED TO ANOTHER HETEROATOM VIA ONE SP3 CARBON
Z is a direct bond, CO or S(O)ₙ group,
B is (CH₂)ₚ,
R¹² and R¹³ each independently represent H or C₁₋₄ alkyl,
or R^{12 and R}¹³ can be taken together with the N atom to which they are attached to form a 4- to 7-membered heterocycle optionally comprising a further hetero moiety selected from NR¹⁶, O and/or S, and which is optionally substituted by one or more C₁₋₄ alkyl,
R¹⁴ and R¹⁵ each independently represent H, C₁₋₁₀ alkyl, C₃₋₁₀ alkenyl, C₃₋₁₀ alkynyl, C₃₋₈ cycloalkyl, aryl or heteroaryl,
or R¹⁴ and R¹⁵ can be taken together with the N atom to which they are attached to form a 4- to 7-membered heterocycle optionally comprising a further hetero moiety selected from NR¹⁶, O and/or S, and which is optionally substituted by one or more C₁₋₄ alkyl,
R¹⁶ is H, C₁₋₆ alkyl, C₃₋₆ cycloalkyl, (C₁₋₄ alkylene)(C₃₋₆ cycloalkyl) or (C₁₋₆ alkylene)aryl,
R⁵ and R⁸ when taken separately are each independently H, C₁₋₆ alkyl,
R⁵ and R⁸ can be taken together with the carbon atoms to which they are joined to form a C₃₋₈ cycloalkyl ring,
R⁶, R⁷, R⁹ and R¹⁰ when taken separately are H,
R⁵ and R⁶ or R⁷ can be taken together with the carbon atoms to which they are joined to form a C₃₋₈ cycloalkyl ring,
X is halogen, C₁₋₄ alkyl, C₁₋₄ alkoxy, C₁₋₄ haloalkyl or C₁₋₄ haloalkoxy,
m is 1 or 2;
n is 0, 1 or 2;
p is 0, 1, 2, 3, 4, 5, 6, 7, 8, 9 or 10;
q is 0 or 1;
“Naturally occuring amino acid substituent” means the □-substituent that occurs in any one of the following natural amino acids, glycine, alanine, valine, leucine, isoleucine, phenylalanine, tryptophan, tyrosine, histidine, serine, threonine, methionine, cysteine, aspartic acid, glutamic acid, asparagine, glutamine, lysine, arginine or proline;
“Heteroaryl” represents an aromatic ring containing up to four heteroatoms independently selected from N, O and S, and if a S atom is present in the ring, it can be present as part of a —S—, S(O)— or —S(O)₂— group, and which may be joined to the remainder of the compound via any available atom(s);
“Heterocycle” is a group containing 1, 2 or 3 rings, and which contains up to 4 ring heteroatoms selected from N, O and S and up to 18 ring carbon atoms;
“Aryl”, including in the definitions of “aryloxy”, etc., means a group comprising a phenyl ring and which may incorporate a further carbocyclic ring fused to said phenyl ring and which may be joined to the remainder of the compound via any available atom(s) (examples of such groups include naphthyl, indanyl, etc.);
“Alkyl”, “alkenyl” and “alkynyl” groups can be linear or branched if the number of carbon atoms allows;
“Cycloalkyl” groups can be polycyclic if the number of carbon atoms allows;
or a pharmaceutically or veterinarily acceptable derivative or prodrug thereof.

2. A substance according to claim 1 wherein the “Ar” ring represents phenyl or pyridyl.

3. A substance according to any preceding claim wherein R¹ when taken alone is OH, CN, halogen, NO₂, NH₂, NY²WY¹ or Het¹.

4. A substance according to any preceding claim wherein R² when taken alone is H.

5. A substance according to claim 1 or 2 wherein R¹ and R² are taken together with the carbon atoms to which they are attached and represent an optionally benzo-fused 5- to 7-membered heteroaryl ring optionally substituted by C₁₋₄ alkyl or C₁₋₄ haloalkyl.

6. A substance according to any preceding claim wherein X is Cl.

7. A substance according to any preceding claim wherein n is 0 and q is 0.

8. A substance according to any preceding claim wherein R³ is H, CN, or C₁₋₆ alkyl (optionally substituted by one or more halogen, OH, C₁₋₆ alkoxy, C₁₋₆ alkoxycarbonyl, C₂₋₆ alkanoyl, C₂₋₆ alkanoyloxy, C₂₋₆ alkyloxycarbonyloxy, NR¹²R¹³, CONR¹²R¹³ and/or NY²WY¹).

9. A substance according to-any preceding claim wherein R⁴ is C₁₋₁₀ alkyl substituted by one or more substituents selected from:
C₂₋₆ alkoxy [substituted by one or more groups selected from OH, NR²⁵R²⁶, CONR²⁵R²⁶, halogen, C₁₋₆ alkoxy, C₂₋₄ alkynyl, C₂₋₄ alkenyl, heteroaryl¹, aryl¹, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O),(C, alkyl), S(O)n(aryl¹), S(O)ₙ(heteroaryl¹), SO₂NR²⁵R²⁶ and cycloalkyl¹],
S(O)ₙC₁₋₆ alkyl [optionally substituted by one or more groups selected from OH, NR²⁵R²⁶, CONR²⁵R²⁶, halogen, C₁₋₆ alkoxy, C₂₋₄ alkynyl, C₂₋₄ alkenyl, heteroaryl¹, aryl¹, COCH₂CN, CO(heteroaryl¹), CO(aryl¹), CO₂(heteroaryl¹), COCH₂(aryl¹), COCH₂(heteroaryl¹), CO₂CH₂(aryl¹), CO₂CH₂(heteroaryl¹), S(O)ₙ(C₁₋₆ alkyl), S(O)n(aryl¹), S(O)n(heteroaryl¹), SO₂NR²⁵R²⁶ and cycloalkyl¹],
aryl²,
CO₂CH₂(heteroaryl¹),
CO₂CH₂(aryl¹),
cycloalkyl¹,
CO(heteroaryl¹),
CO(aryl¹),
OCO(aryl¹),
OCO(heteroaryl¹),
OCO(C₁₋₆ alkyl),
OCOCH₂CN,
CO₂(heteroaryl¹),
CO₂(aryl¹),
COCH₂(heteroaryl¹),
S(O)ₙaryl¹,
S(O)ₙCH₂aryl¹,
S(O)n(heteroaryl¹),
S(O)ₙCH₂(heteroaryl¹),
NHSO₂aryl¹,
NHSO₂(C₁₋₆ alkyl),
NHSO₂(heteroaryl¹),
NHSO₂CH₂(heteroaryl¹),
NHSO₂CH₂(aryl¹),
NHCOaryl¹,
NHCO(C₁alkyl),
NHCONHaryl¹,
NHCONH(C₁₋₆ alkyl),
NHCOheteroaryl¹,
NHCONHheteroaryl¹,
NHCO₂(aryl¹),
NHCO₂(C₁alkyl),
NHCO₂(heteroaryl¹),
aryl²oxy,
heteroaryl¹ oxy,
C₁₋₆ alkoxycarbonyl substituted by C₁₋₆ alkyl, aryl, C₁₋₄ alkoxy, CH₂(aryl¹), C₁₋₄ haloalkyl, halogen, OH, CN or NR²⁵R²⁶,
C₂₋₆ alkanoyl substituted by C₁₋₆ alkyl, aryl, C₁₋₆ alkoxy, CH₂(aryl¹), C₁₋₄ haloalkyl, halogen, OH, CN or NR²⁵R²⁶,
C₂₋₆ alkanoyloxy substituted by C₁₋₄ alkyl, aryl, C₁₋₆ alkoxy, CH₂(aryl¹), C₁₋₄ haloalkyl, halogen, OH, CN or NR²⁵R²⁶,
cycloalkyl¹oxy,
COcycloalkyl¹,
heterocycle substituted by one or more substituent selected from C₁₋₆ alkyl(substituted by OH), CONR²⁵R²⁵, CH₂CONR²⁵R²⁶, NR²⁵R²⁶, NHCONR²⁵R²⁶ alkyl), SO₂NR²⁵R²⁶SO₂(C₁₋₆ alkyl), CO₂(C₁alkyl), CH₂CO₂(C₁₋₆ alkyl), OCH₂CO₂(C₁₋₆ alkyl), aryl, heterocyclyl, aryloxy, aryl(CH₂)oxy, aryl(CH₂), CN and C₃₋₇ cycloalkyl, heterocyclyloxy substituted by one or more substituent selected from C₁₋₆ alkyl(substituted by OH), CONR²⁵R²⁶,CH₂CONR²⁵R²⁶, NR²⁵R²⁶, NHCONR²⁵R²⁶ alkyl), SO₂NR²⁵R²⁶, SO₂(C₁l6 alkyl), CO₂(C₁₋₆ alkyl), CH₂CO₂(C₁₋₆ alkyl), OCH₂CO₂(C₁₋₆ alkyl), aryl, heterocyclyl, aryloxy, aryl(CH₂)oxy, aryi(CH₂), CN and C₃₋₇ cycloalkyl,

10. A substance according to any preceding claim wherein R⁵, R⁶, R⁷, R⁸ R⁹ and R¹⁰ are each taken separately and are all H.

11. A substance according to any preceding claim wherein the “Ar” ring represents a group of formula:

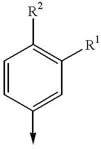

12. A substance according to any preceding claim wherein R³ is H, CH₃, C₂H₅, i-C₃H₇, n-C₃H₇ or CH₂OCH₃.

13. A substance according to any preceding claim except claim 5 wherein R¹ is OH, CN, I, Cl, NH₂, NO₂, optionally benzo-fused heteroaryl, NHSO₂Y¹, NHCOY¹ or NHCO₂Y¹.

14. A substance according to any preceding claim wherein R⁴ is C₁₋₁₀ alkyl substituted by cycloalkyl¹.

15. A substance according to any preceding claim except claims 3, 4 and 13 wherein R¹ and R2 are taken together with the carbon atoms to which they are attached are a 5-membered heteroaryl moiety optionally substituted by C₁₋₄ alkyl or C₁₋₄ haloalkyl.

16. A substance according to any preceding claim wherein R³ is CH₃ or C₂H₅.

17. A substance according to any preceding claim except claims 5 and 15 wherein R¹ when taken alone is OH, CN, I, Cl, NH₂, NO₂, 1,2,3-triazolyl, 1,2,4-triazolyl, imidazol-2-yl, pyridin-2-yl, thien-2-yl, imidazol-4-yi, benzimidazol-2-yl, NHSO₂(C₁₋₆ alkyl), NHSO₂(C₁₋₆ alkyl substituted by methoxy, CONH₂, OH, CO₂(C₂₋₆ alkyl), phthalimido, NH₂ or halogen), NHSO₂NH₂, NHSO₂NH(C₁₋₆ alkyl), NHSO₂N(C₁₋₆ alkyl)₂, NHSO₂Het₁ₐ, NHCO(C₁₋₆ alkyl) or NHCO₂(C₁₋₆ alkyl).

18. A substance according to claim 17 wherein R¹ is OH, NHSO₂CH₃, NHSO₂C₂H₅, NHSO₂(n-C₃H₇), NHSO₂(i-C₃H₇), NHSO₂(n-C₄H₇), NHSO₂NH(i-C₃H₇), NHSO₂(N-methylimidazol-4-yl), NHSO₂(CH₂)₂OCH₃, NHSO₂(CH₂)₂OH, 1,2,4-triazolyl or imidazol-2-yl.

19. A substance according to claim 18 wherein R¹ is OH, NHSO₂CH₃, NHSO₂C₂H₅ or imidazol-2-yl.

20. A substance according to claim 15 wherein R¹ and R2 when taken together with the carbon atoms to which they are attached are an imidazole group optionally 2-substituted by CF₃.

21. A substance according to any preceding claim wherein R⁴ is C₂₋₄ alkyl substituted by cycloalkyl¹.

22. A substance according to any preceding claim wherein R⁴ is propyl substituted by cycloalkyl¹.

23. A substance according to any preceding claim wherein R⁴ is propyl substituted by a C₃₋₁₀ carbocyclic system with one or two rings and which is substituted by OH.

24. A substance according to any preceding claim wherein R⁴ is propyl substituted by (cyclohexyl substituted by OH).

25. A substance according to any preceding claim wherein R⁴ is (1-hydroxycyclohexyl)prop-3-yl.

26. A substance according to claim 1 which has the following relative stereochemistry:

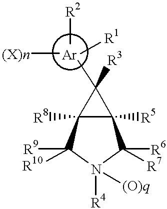

27. A substance according to claim 1 which is selected from the compounds of the Examples as described herein, and the salts and prodrugs thereof.

28. A pharmaceutical or veterinary composition comprising a substance according to any one of the preceding claims, and a pharmacutically or veterinarily acceptable carrier.

29. A substance according to any of claims 1 to 26 for use in medicine.

30. A substance according to any of claims 1 to 26 for use as a medicament useful for the treatment of an opiate-mediated disease or condition.

31. The use of a substance according to any one of claims 1 to 26 in the manufacture of a medicament for the treatment of a disease or condition mediated by opiate receptors.

32. A method of treatment of a condition mediated by an opiate receptor or receptors comprising administration of a therapeutically active amount of a substance according to any one of claims 1 to 26.

33. A process for the preparation of a substance according to claim 1 which comprises:
(a) for compounds of formula I in which q is 0 and R¹ represents NY²WY¹, reacting a compound of formula II, 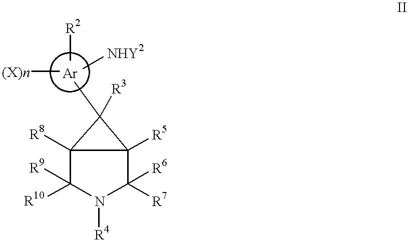
with a compound of formula II,
Z¹-WY¹ III
wherein Z¹ is a suitable leaving group, such as halogen or Y¹SO₂O—;
(b) for compounds of formula I in which q is 0 and R⁶ and R⁷ both represent H, reduction of a compound of formula IV, 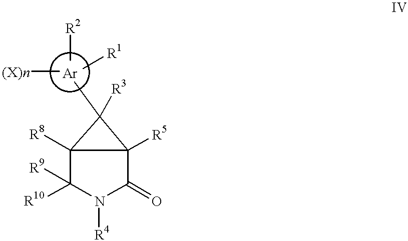
using a suitable reducing agent;
(c) for compounds of formula I in which q is 0 and R⁹ and R¹⁰ both represent H, reduction of a compound of formula V, 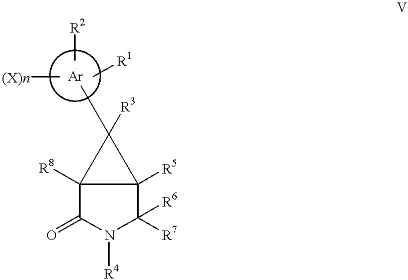
using a suitable reducing agent;
(d) for compounds of formula I in which q is 0 and R¹ and R2 are attached to adjacent carbon atoms and are taken together with the carbon atoms to which they are attached to represent Het^{1a}, in which Het^{1a} represents an imidazolo unit, reaction of a corresponding compound of formula VI, 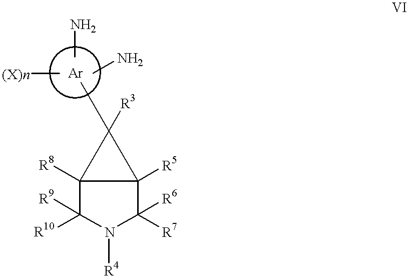
with a compound of formula VII,
R^{y}CO₂H VII
wherein R^{y} represents H or any of the optional substituents on Het^{1a} (as defined above), preferably H, C₁₋₄ alkyl or C₁₋₄ haloalkyl;
(e) where q is 0, reacting a compound of formula VIII, 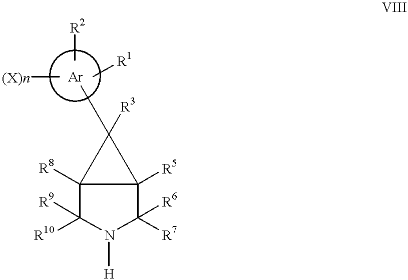
with a compound of formula IX,
R⁴-Lg IX
wherein Lg is a leaving group;
(f) for compounds of formula I in which q is 0 and R⁶, R⁷, R⁹ and R¹⁰ are all H, reduction of a compound of formula X, 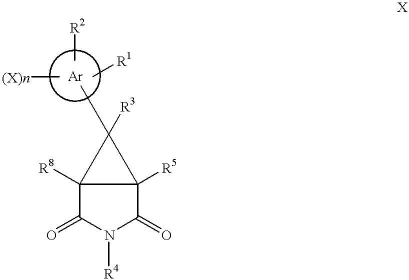
with a suitable reducing agent;
(g) for compounds of formula I in which q is 0 and R¹ represents OH, reacting a compound of formula II, where Y² is H, as defined above, with fluoroboric acid and isoamyl nitrite;
(h) for compounds of formula I in which q is 0 and R¹ represents Cl, reacting a compound of formula II, where Y² is H, as defined above, with sodium nitrite in the presence of dilute acid, followed by reaction with copper (I) chloride in the presence of concentrated acid;
(i) for compounds of formula I in which q is 1, reacting a compound of formula I where q is 0 with a suitable oxidising agent such as aqueous hydrogen peroxide; or
(j) for compounds of formula I where q is 0, by reduction of a corresponding compound of formula XXXI, 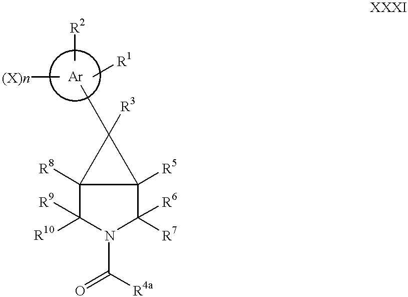
where R^{4a}CH₂ takes the same meaning as R⁴ as defined above,
(K) for compounds of formula (I) where q is 0, reductive amination reaction of the amine of formula VIII above with an aldehyde of formula R^{4a}-CHO wherein R^{4a}CH₂ takes the same meaning as R⁴ as defined above,
and where desired or necessary converting the resulting compound of formula I into a pharmaceutically or veterinarily acceptable derivative or vice versa.

34. A compound of formula II, IV, V, VI, X, X^{a}, XI, XII, XXI, XXII, XXIII, XXIV, XXIX, XXIXa, XXX, or XXXI, or salt thereof, as described herein.
